(19) United States
(12) Patent Application Publication
(10) Pub. No.: US 2025/0140345 A1
(43) Pub. Date: May 1, 2025

(54) CYTOKINE GENE EXPRESSION SIGNATURES

(71) Applicant: BostonGene Corporation, Waltham, MA (US)

(72) Inventors: Sofya Kust, Yerevan (AM); Anastasia Zotova, Moscow (RU); Siune Ambarian, Yerevan (AM); Dmitrii Tabakov, Yerevan (AM); Ekaterina Postovalova, Waltham, MA (US); Olga Kudriashova, Moscow (RU); Elena Ocheredko, Yerevan (AM); Eleonora Belykh, Vologda (RU); Mariia Sorokina, Moscow (RU); Alexander Bagaev, Waltham, MA (US); Maria Savchenko, Yerevan (AM); Natalia Mikheecheva, Yerevan (AM); Kirill Ovchinnikov, Yerevan (AM)

(73) Assignee: BostonGene Corporation, Waltham, MA (US)

(21) Appl. No.: 18/835,974

(22) PCT Filed: Mar. 3, 2023
(86) PCT No.: PCT/US2023/014459

§ 371 Date: Aug. 5, 2024

Related U.S. Application Data
(60) Provisional application No. 63/316,471, filed on Mar. 4, 2022.

(51) Int. Cl. G16B 25/10; G16H 20/10; G16H 50/20

(52) U.S. Cl. CPC G16B 25/10; G16H 20/10; G16H 50/20

(57) ABSTRACT

Aspects of the disclosure relate to methods, systems, computer-readable storage media, and graphical user interfaces (GUIs) that are useful for characterizing subjects having certain cancers, for example solid tumor cancers or blood cancers. The disclosure is based, in part, on methods for determining a cytokine signature of a subject and the subject's prognosis and/or likelihood of responding to a therapy based upon the cytokine signature determination.

### RELATED APPLICATIONS

This application claims the benefit under 35 U.S.C. § 119(e) of the filing date of U.S. provisional Application Ser. No. 63/316,471, filed Mar. 4, 2022, the entire contents of which are incorporated by reference herein.

### BACKGROUND

Cytokines are small, secreted proteins that mediate a variety of effects in the immune system, for example control of inflammation. In cancer, cytokines may influence the tumor microenvironmental (TME) landscape by immunomodulation, or promote tumor growth and stroma remodeling. Cytokines can also have tumor-suppressive effects by inhibiting tumor growth and angiogenesis processes or by activating the anti-tumor immune response.

### SUMMARY

Aspects of the disclosure relate to methods systems, and computer-readable storage media, which are useful for characterizing cytokine expression and function in subjects having certain cancers, for example solid tumor cancers (STCs) and blood cancers (BCs). The disclosure is based, in part, on methods for generating a cytokine signature of a subject having an STC or BC by using gene expression data obtained from the subject. In some embodiments, the cytokine signature is indicative of one or characteristics of the subject (or the subject's cancer), for example the likelihood the subject will respond to a particular therapy (e.g., a cytokine therapy, etc.) or the likelihood a subject will have a good prognosis.

Accordingly, in some aspects, the disclosure provides a method for generating a cytokine signature for a subject having solid tumor cancer, the method comprising: using at least one computer hardware processor to perform obtaining RNA expression data for the subject, the RNA expression data for the subject indicating RNA expression levels for at least some genes in each group of at least some of a plurality of solid tumor cancer (STC) gene groups, the plurality of STC gene groups comprising (i) STC gene groups associated with pro-tumor effects, (ii) STC gene groups associated with anti-tumor effects, and (iii) STC gene groups associated with B cell effects; obtaining RNA expression data for a cohort of subjects having a same type of STC as the subject, the RNA expression data for the cohort indicating RNA expression levels for the at least some of the genes in each group of the at least some of the plurality of STC gene groups; generating, using the RNA expression data for the subject and the RNA expression data for the cohort of subjects, the cytokine signature for the subject, the cytokine signature comprising normalized STC gene group scores for respective STC gene groups in the at least some of the plurality of STC gene groups, the generating comprising: determining, using the RNA expression data for the subject, initial STC gene group scores for (i) one or more STC gene groups associated with pro-tumor effects, (ii) one or more STC gene groups associated with anti-tumor effects, and/or (iii) one or more STC gene groups associated with B cell effects; and determining the normalized STC gene group scores of the cytokine signature by normalizing the initial STC gene group scores using the RNA expression data for the cohort of subjects.

In some embodiments, the STC gene groups associated with pro-tumor effects comprise one or more gene groups from among: myeloid inflammation groups, Type 2 response groups, immune suppression groups, tumor promotion groups, and stroma activation groups, and determining the initial STC gene group scores comprises, determining STC gene group scores for the one or more gene groups from among the myeloid inflammation groups, the Type 2 response groups, the immune suppression groups, the tumor promotion groups, and the stroma activation groups.

In some embodiments, the STC gene groups associated with anti-tumor effects comprise one or more gene groups from among: Type 1 response groups and tumor suppression groups, and determining the initial STC gene group scores comprises, determining STC gene group scores for the one or more gene groups from among the Type 1 response groups and the tumor suppression groups.

In some embodiments, the STC gene groups associated with B cell effects comprise one or more gene groups from among: B cell function group and immune cell recruitment groups, and determining the initial STC gene group scores comprises, determining STC gene group scores for the one or more gene groups from among the B cell function group and immune cell recruitment groups.

In some embodiments, the myeloid inflammation groups comprise the pro-inflammatory cytokines group which comprises at least three genes listed in Table 1 for the pro-inflammatory cytokines group, and determining the initial STC group scores comprises: obtaining, from the RNA expression data for the subject, RNA expression levels for the at least three genes listed in Table 1; and determining an initial STC group score for the pro-inflammatory cytokines group using the RNA expression levels for the at least three genes listed in Table 1.

In some embodiments, the myeloid inflammation groups comprise the neutrophil recruitment and activation group which comprises at least three genes listed in Table 1 for the neutrophil recruitment and activation group, and determining the initial STC group scores comprises obtaining, from the RNA expression data for the subject, RNA expression levels for the at least three genes listed in Table 1; and determining an initial STC group score for the neutrophil recruitment and activation group using the RNA expression levels for the at least three genes listed in Table 1.

In some embodiments, the Type 2 response groups comprise the Th2 response group which comprises at least three genes listed in Table 1 for the Th2 response group, and determining the initial STC group scores comprises: obtaining, from the RNA expression data for the subject, RNA expression levels for the at least three genes listed in Table 1; and determining an initial STC group score for the Th2 response group using the RNA expression levels for the at least three genes listed in Table 1.

In some embodiments, the Type 2 response groups comprise the M2 polarization group which comprises at least three genes listed in Table 1 for the M2 polarization group, and determining the initial STC group scores comprises: obtaining, from the RNA expression data for the subject, RNA expression levels for the at least three genes listed in Table 1; and determining an initial STC group score for the M2 polarization group using the RNA expression levels for the at least three genes listed in Table 1.

In some embodiments, the Type 2 response groups comprise the eosinophil/basophil recruitment group which comprises at least three genes listed in Table 1 for the eosinophil/basophil recruitment group, and determining the initial STC group scores comprises: obtaining, from the RNA expression data for the subject, RNA expression levels for the at least three genes listed in Table 1; and determining an initial STC group score for the eosinophil/basophil recruitment group using the RNA expression levels for the at least three genes listed in Table 1.

In some embodiments, the Type 2 response groups comprise the eosinophil/basophil activation group which comprises at least three genes listed in Table 1 for the eosinophil/basophil activation group, and determining the initial STC group scores comprises: obtaining, from the RNA expression data for the subject, RNA expression levels for the at least three genes listed in Table 1; and determining an initial STC group score for the eosinophil/basophil activation group using the RNA expression levels for the at least three genes listed in Table 1.

In some embodiments, the immune suppression groups comprise the stromal suppressive factors group which comprises at least three genes listed in Table 1 for the stromal suppressive factors group, and determining the initial STC group scores comprises: obtaining, from the RNA expression data for the subject, RNA expression levels for the at least three genes listed in Table 1; and determining an initial STC group score for the stromal suppressive factors group using the RNA expression levels for the at least three genes listed in Table 1.

In some embodiments, the immune suppression groups comprise the myeloid suppressive factors group which comprises at least three genes listed in Table 1 for the myeloid suppressive factors group, and determining the initial STC group scores comprises: obtaining, from the RNA expression data for the subject, RNA expression levels for the at least three genes listed in Table 1; and determining an initial STC group score for the myeloid suppressive factors group using the RNA expression levels for the at least three genes listed in Table 1.

In some embodiments, the immune suppression groups comprise the CTL exclusion group which comprises at least three genes listed in Table 1 for the CTL exclusion group, and determining the initial STC group scores comprises: obtaining, from the RNA expression data for the subject, RNA expression levels for the at least three genes listed in Table 1; and determining an initial STC group score for the CTL exclusion group using the RNA expression levels for the at least three genes listed in Table 1.

In some embodiments, the immune suppression groups comprise the Treg polarization group which comprises at least three genes listed in Table 1 for the Treg polarization group, and determining the initial STC group scores comprises: obtaining, from the RNA expression data for the subject, RNA expression levels for the at least three genes listed in Table 1; and determining an initial STC group score for the Treg polarization group using the RNA expression levels for the at least three genes listed in Table 1.

In some embodiments, the tumor promotion groups comprise the tumor growth promotion group which comprises at least three genes listed in Table 1 for the tumor growth promotion group, and determining the initial STC group scores comprises: obtaining, from the RNA expression data for the subject, RNA expression levels for the at least three genes listed in Table 1; and determining an initial STC group score for the tumor growth promotion group using the RNA expression levels for the at least three genes listed in Table 1.

In some embodiments, the tumor promotion groups comprise the induction of EMT group which comprises at least three genes listed in Table 1 for the induction of EMT group, and determining the initial STC group scores comprises: obtaining, from the RNA expression data for the subject, RNA expression levels for the at least three genes listed in Table 1; and determining an initial STC group score for the induction of EMT group using the RNA expression levels for the at least three genes listed in Table 1.

In some embodiments, the tumor promotion groups comprise the metastasis promotion group which comprises at least three genes listed in Table 1 for the metastasis promotion group, and determining the initial STC group scores comprises: obtaining, from the RNA expression data for the subject, RNA expression levels for the at least three genes listed in Table 1; and determining an initial STC group score for the metastasis promotion group using the RNA expression levels for the at least three genes listed in Table 1.

In some embodiments, the stroma activation groups comprise the angiogenesis induction group which comprises at least three genes listed in Table 1 for the angiogenesis induction group, and determining the initial STC group scores comprises: obtaining, from the RNA expression data for the subject, RNA expression levels for the at least three genes listed in Table 1; and determining an initial STC group score for the angiogenesis induction group using the RNA expression levels for the at least three genes listed in Table 1.

In some embodiments, the stroma activation groups comprise the CAF recruitment group which comprises at least three genes listed in Table 1 for the CAF recruitment group, and determining the initial STC group scores comprises: obtaining, from the RNA expression data for the subject, RNA expression levels for the at least three genes listed in Table 1; and determining an initial STC group score for the CAF recruitment group using the RNA expression levels for the at least three genes listed in Table 1.

In some embodiments, the Type 1 response groups comprise the CTL and Th1 cells activation group which comprises at least three genes listed in Table 1 for the CTL and Th1 cells activation group, and determining the initial STC group scores comprises: obtaining, from the RNA expression data for the subject, RNA expression levels for the at least three genes listed in Table 1; and determining an initial STC group score for the CTL and Th1 cells activation group using the RNA expression levels for the at least three genes listed in Table 1.

In some embodiments, the Type 1 response groups comprise the M1 polarization group which comprises at least three genes listed in Table 1 for the M1 polarization group, and determining the initial STC group scores comprises: obtaining, from the RNA expression data for the subject, RNA expression levels for the at least three genes listed in Table 1; and determining an initial STC group score for the M1 polarization group using the RNA expression levels for the at least three genes listed in Table 1.

In some embodiments, the Type 1 response groups comprise the TLS formation group which comprises at least three genes listed in Table 1 for the TLS formation group, and determining the initial STC group scores comprises: obtaining, from the RNA expression data for the subject, RNA expression levels for the at least three genes listed in Table 1; and determining an initial STC group score for the TLS formation group using the RNA expression levels for the at least three genes listed in Table 1.

In some embodiments, the tumor suppression groups comprise the tumor growth arrest group which comprises at least three genes listed in Table 1 for the tumor growth arrest group, and determining the initial STC group scores comprises: obtaining, from the RNA expression data for the subject, RNA expression levels for the at least three genes listed in Table 1; and determining an initial STC group score for the tumor growth arrest group using the RNA expression levels for the at least three genes listed in Table 1.

In some embodiments, the tumor suppression groups comprise the metastasis inhibition group which comprises at least three genes listed in Table 1 for the metastasis inhibition group, and determining the initial STC group scores comprises: obtaining, from the RNA expression data for the subject, RNA expression levels for the at least three genes listed in Table 1; and determining an initial STC group score for the metastasis inhibition group using the RNA expression levels for the at least three genes listed in Table 1.

In some embodiments, the tumor suppression groups comprise the angiogenesis inhibition group which comprises at least three genes listed in Table 1 for the angiogenesis inhibition group, and determining the initial STC group scores comprises: obtaining, from the RNA expression data for the subject, RNA expression levels for the at least three genes listed in Table 1; and determining an initial STC group score for the angiogenesis inhibition group using the RNA expression levels for the at least three genes listed in Table 1.

In some embodiments, the B cell function group comprises the B cell activation group which comprises at least three genes listed in Table 1 for the B cell activation group, and determining the initial STC group scores comprises: obtaining, from the RNA expression data for the subject, RNA expression levels for the at least three genes listed in Table 1; and determining an initial STC group score for the B cell activation group using the RNA expression levels for the at least three genes listed in Table 1.

In some embodiments, the immune cell recruitment groups comprise the lymphocyte recruitment group which comprises at least three genes listed in Table 1 for the lymphocyte recruitment group, and determining the initial STC group scores comprises: obtaining, from the RNA expression data for the subject, RNA expression levels for the at least three genes listed in Table 1; and determining an initial STC group score for the lymphocyte recruitment group using the RNA expression levels for the at least three genes listed in Table 1.

In some embodiments, the immune cell recruitment groups comprise the macrophage and DC recruitment group which comprises at least three genes listed in Table 1 for the macrophage and DC recruitment group, and determining the initial STC group scores comprises: obtaining, from the RNA expression data for the subject, RNA expression levels for the at least three genes listed in Table 1; and determining an initial STC group score for the macrophage and DC recruitment group using the RNA expression levels for the at least three genes listed in Table 1.

In some embodiments, determining the initial STC gene group scores comprises performing single-sample gene set enrichment analysis (ssGSEA) on the RNA expression data for the subject.

In some embodiments, determining the initial STC gene group scores comprises performing single-sample gene set enrichment analysis (ssGSEA) using the RNA expression levels for the at least three genes of the pro-inflammatory cytokines group listed in Table 1.

In some embodiments, the method further comprises determining that each normalized STC gene group score is (i) below a first threshold value, (ii) between the first threshold value and a second threshold value, or (iii) above the second threshold value, wherein each of the first and second threshold values is determined using the RNA expression data for the cohort of subjects, and identifying each respective normalized STC gene group score as “low” when the respective normalized STC gene group score is below the first threshold value, identifying each respective normalized STC gene group score as “medium” when the respective gene group score is between the first threshold value and the second threshold value, or identifying each respective normalized STC gene group score as “high” when the respective gene group score is above the second threshold value.

In some embodiments, the method further comprises generating a visualization of the cytokine signature, the generating comprising generating a graphical user interface (GUI) having a plurality of GUI elements, each of the GUI elements representing a respective normalized STC gene group score part of the cytokine signature. In some embodiments, a particular GUI element of the plurality GUI elements represents the respective normalized STC gene group score via a visual characteristic of the GUI element. In some embodiments, the visual characteristic is selected from the group consisting of a color, size, and font.

In some embodiments, the method further comprises identifying at least one therapeutic agent for administration to the subject using the cytokine signature.

In some embodiments, the method further comprises administering the at least one identified therapeutic agent to the subject.

In some embodiments, the at least one therapeutic agent is an immune checkpoint inhibitor (ICI) or a tyrosine kinase inhibitor (TKI).

In some embodiments, the at least one identified therapeutic agent is an immune checkpoint inhibitor when the subject has been identified as having a “high” normalized STC gene group score for one or more of the following gene groups: immune cell recruitment groups, B cell response groups, or Type 1 response groups. In some embodiments, the at least one identified therapeutic agent is an immune checkpoint inhibitor (ICI) when the subject is identified as having a “low normalized STC gene group score for one or more of the following gene groups: tumor suppression gene groups, immune suppression gene groups, or stromal activation gene groups.

In some embodiments, the at least one identified therapeutic agent is a TKI when the subject has been identified as having a “high” normalized STC gene group score for one or more of the following gene groups: myeloid inflammation gene groups, or tumor suppression gene groups. In some embodiments, the at least one identified therapeutic agent is a TKI when the subject is identified as having a “low normalized STC gene group score for one or more of the following gene groups: Type 1 response gene groups or stromal activation groups.

In some aspects, the disclosure provides a method for generating a cytokine signature for a subject having blood cancer, the method comprising using at least one computer hardware processor to perform: obtaining RNA expression data for the subject, the RNA expression data for the subject indicating RNA expression levels for at least some genes in each group of at least some of a plurality of blood cancer (BC) gene groups, the plurality of BC gene groups comprising (i) BC gene groups associated with pro-tumor effects, (ii) BC gene groups associated with anti-tumor effects, and (iii) BC gene groups associated with B cell effects; obtaining RNA expression data for a cohort of subjects having a same type of BC as the subject, the RNA expression data for the cohort indicating RNA expression levels for the at least some of the genes in each group of the at least some of the plurality of BC gene groups; generating, using the RNA expression data for the subject and the RNA expression data for the cohort of subjects, the cytokine signature for the subject, the cytokine signature comprising normalized BC gene group scores for respective BC gene groups in the at least some of the plurality of BC gene groups, the generating comprising: determining, using the RNA expression data for the subject, initial BC gene group scores for (i) one or more BC gene groups associated with pro-tumor effects, (ii) one or more BC gene groups associated with anti-tumor effects, and/or (iii) one or more BC gene groups associated with B cell effects; and determining the normalized BC gene group scores of the cytokine signature by normalizing the initial BC gene group scores using the RNA expression data for the cohort of subjects.

In some embodiments, the BC gene groups associated with pro-tumor effects comprise one or more gene groups from among: tumor promotion groups, immune suppression groups, and stroma activation groups, and determining the initial BC gene group scores comprises, determining BC gene group scores for the one or more gene groups from among the tumor promotion groups, immune suppression groups, and the stroma activation groups.

In some embodiments, the BC gene groups associated with anti-tumor effects comprise one or more gene groups from among: Type 1 response groups, and tumor suppression groups, and determining the initial BC gene group scores comprises, determining BC gene group scores for the one or more gene groups from among the Type 1 response groups, and the tumor suppression groups.

In some embodiments, the BC gene groups associated with B cell effects comprise one or more gene groups from among: a pro-inflammatory cytokines group, a pro-inflammatory cytokines FL group, a myeloid cell recruitment group, and a myeloid cell recruitment FL group, and determining the initial BC gene group scores comprises, determining BC gene group scores for the one or more gene groups from among the pro-inflammatory cytokines group, pro-inflammatory cytokines FL group, myeloid cell recruitment group, and myeloid cell recruitment FL group.

In some embodiments, the tumor promotion groups comprise the lymphoma cell pro-survival group which comprises at least three genes listed in Table 2 for the lymphoma cell pro-survival group, and determining the initial BC group scores comprises: obtaining, from the RNA expression data for the subject, RNA expression levels for the at least three genes listed in Table 2; and determining an initial BC group score for the lymphoma cell pro-survival group using the RNA expression levels for the at least three genes listed in Table 2.

In some embodiments, the tumor promotion groups comprise the cancer promoting inflammation group which comprises at least three genes listed in Table 2 for the cancer promoting inflammation group, and determining the initial BC group scores comprises: obtaining, from the RNA expression data for the subject, RNA expression levels for the at least three genes listed in Table 2; and determining an initial BC group score for the cancer promoting inflammation group using the RNA expression levels for the at least three genes listed in Table 2.

In some embodiments, the tumor promotion groups comprise the cancer promoting inflammation FL group which comprises at least three genes listed in Table 2 for the cancer promoting inflammation FL group, and determining the initial BC group scores comprises: obtaining, from the RNA expression data for the subject, RNA expression levels for the at least three genes listed in Table 2; and determining an initial BC group score for the cancer promoting inflammation FL group using the RNA expression levels for the at least three genes listed in Table 2.

In some embodiments, the tumor promotion groups comprise the lymphoma dissemination group which comprises at least three genes listed in Table 2 for the lymphoma dissemination group, and determining the initial BC group scores comprises: obtaining, from the RNA expression data for the subject, RNA expression levels for the at least three genes listed in Table 2; and determining an initial BC group score for the lymphoma dissemination group using the RNA expression levels for the at least three genes listed in Table 2.

In some embodiments, the immune suppression groups comprise the Treg recruitment and function group which comprises at least three genes listed in Table 2 for the Treg recruitment and function group, and determining the initial BC group scores comprises: obtaining, from the RNA expression data for the subject, RNA expression levels for the at least three genes listed in Table 2; and determining an initial BC group score for the Treg recruitment and function group using the RNA expression levels for the at least three genes listed in Table 2.

In some embodiments, the immune suppression groups comprise the immunosuppressive factors group which comprises at least three genes listed in Table 2 for the immunosuppressive factors group, and determining the initial BC group scores comprises: obtaining, from the RNA expression data for the subject, RNA expression levels for the at least three genes listed in Table 2; and determining an initial BC group score for the immunosuppressive factors group using the RNA expression levels for the at least three genes listed in Table 2.

In some embodiments, the immune suppression groups comprise the immunosuppressive factors FL group which comprises at least three genes listed in Table 2 for the immunosuppressive factors FL group, and determining the initial BC group scores comprises: obtaining, from the RNA expression data for the subject, RNA expression levels for the at least three genes listed in Table 2; and determining an initial BC group score for the immunosuppressive factors FL group using the RNA expression levels for the at least three genes listed in Table 2.

In some embodiments, the immune suppression groups comprise the M2 polarization and response group which comprises at least three genes listed in Table 2 for the M2 polarization and response group, and determining the initial BC group scores comprises: obtaining, from the RNA expression data for the subject, RNA expression levels for the at least three genes listed in Table 2; and determining an initial BC group score for the M2 polarization and response group using the RNA expression levels for the at least three genes listed in Table 2.

In some embodiments, the immune suppression groups comprise the M2 polarization and response FL group which comprises at least three genes listed in Table 2 for the M2 polarization and response FL group, and determining the initial BC group scores comprises: obtaining, from the RNA expression data for the subject, RNA expression levels for the at least three genes listed in Table 2; and determining an initial BC group score for the M2 polarization and response FL group using the RNA expression levels for the at least three genes listed in Table 2.

In some embodiments, the stroma activation groups comprise the stroma and angiogenesis activation group which comprises at least three genes listed in Table 2 for the stroma and angiogenesis activation group, and determining the initial BC group scores comprises: obtaining, from the RNA expression data for the subject, RNA expression levels for the at least three genes listed in Table 2; and determining an initial BC group score for the stroma and angiogenesis activation group using the RNA expression levels for the at least three genes listed in Table 2.

In some embodiments, the stroma activation groups comprise the stroma and angiogenesis activation FL group which comprises at least three genes listed in Table 2 for the stroma and angiogenesis activation FL group, and determining the initial BC group scores comprises: obtaining, from the RNA expression data for the subject, RNA expression levels for the at least three genes listed in Table 2; and determining an initial BC group score for the stroma and angiogenesis activation FL group using the RNA expression levels for the at least three genes listed in Table 2.

In some embodiments, the Type 1 response groups comprise the T cell recruitment group which comprises at least three genes listed in Table 2 for the T cell recruitment group, and determining the initial BC group scores comprises: obtaining, from the RNA expression data for the subject, RNA expression levels for the at least three genes listed in Table 2; and determining an initial BC group score for the T cell recruitment group using the RNA expression levels for the at least three genes listed in Table 2.

In some embodiments, the Type 1 response groups comprise the cancer inhibiting inflammation group which comprises at least three genes listed in Table 2 for the cancer inhibiting inflammation group, and determining the initial BC group scores comprises: obtaining, from the RNA expression data for the subject, RNA expression levels for the at least three genes listed in Table 2; and determining an initial BC group score for the cancer inhibiting inflammation group using the RNA expression levels for the at least three genes listed in Table 2.

In some embodiments, the Type 1 response groups comprise the Th1/M1 polarization and response group which comprises at least three genes listed in Table 2 for the Th1/M1 polarization and response group, and determining the initial BC group scores comprises: obtaining, from the RNA expression data for the subject, RNA expression levels for the at least three genes listed in Table 2; and determining an initial BC group score for the Th1/M1 polarization and response group using the RNA expression levels for the at least three genes listed in Table 2.

In some embodiments, the Type 1 response groups comprise the Th1/M1 polarization and response FL group which comprises at least three genes listed in Table 2 for the Th1/M1 polarization and response FL group, and determining the initial BC group scores comprises: obtaining, from the RNA expression data for the subject, RNA expression levels for the at least three genes listed in Table 2; and determining an initial BC group score for the Th1/M1 polarization and response FL group using the RNA expression levels for the at least three genes listed in Table 2.

In some embodiments, the tumor suppression groups comprise the invasion and angiogenesis inhibition group which comprises at least three genes listed in Table 2 for the invasion and angiogenesis inhibition group, and determining the initial BC group scores comprises: obtaining, from the RNA expression data for the subject, RNA expression levels for the at least three genes listed in Table 2; and determining an initial BC group score for the invasion and angiogenesis inhibition group using the RNA expression levels for the at least three genes listed in Table 2.

In some embodiments, the tumor suppression groups comprise the invasion and angiogenesis inhibition FL group which comprises at least three genes listed in Table 2 for the invasion and angiogenesis inhibition FL group, and determining the initial BC group scores comprises: obtaining, from the RNA expression data for the subject, RNA expression levels for the at least three genes listed in Table 2; and determining an initial BC group score for the invasion and angiogenesis inhibition FL group using the RNA expression levels for the at least three genes listed in Table 2.

In some embodiments, the pro-inflammatory cytokines group comprises at least three genes listed in Table 2 for the pro-inflammatory cytokines group, and determining the initial BC group scores comprises: obtaining, from the RNA expression data for the subject, RNA expression levels for the at least three genes listed in Table 2; and determining an initial BC group score for the pro-inflammatory cytokines group using the RNA expression levels for the at least three genes listed in Table 2.

In some embodiments, the pro-inflammatory cytokines FL group comprises at least three genes listed in Table 2 for the pro-inflammatory cytokines FL group, and determining the initial BC group scores comprises: obtaining, from the RNA expression data for the subject, RNA expression levels for the at least three genes listed in Table 2; and determining an initial BC group score for the pro-inflammatory cytokines FL group using the RNA expression levels for the at least three genes listed in Table 2.

In some embodiments, the myeloid cell recruitment group comprises at least three genes listed in Table 2 for the myeloid cell recruitment group, and determining the initial BC group scores comprises: obtaining, from the RNA expression data for the subject, RNA expression levels for the at least three genes listed in Table 2; and determining an initial BC group score for the myeloid cell recruitment group using the RNA expression levels for the at least three genes listed in Table 2.

In some embodiments, the myeloid cell recruitment FL group comprises at least three genes listed in Table 2 for the myeloid cell recruitment FL group, and determining the initial BC group scores comprises: obtaining, from the RNA expression data for the subject, RNA expression levels for the at least three genes listed in Table 2; and determining an initial BC group score for the myeloid cell recruitment FL group using the RNA expression levels for the at least three genes listed in Table 2.

In some embodiments, determining the initial BC gene group scores comprises performing single-sample gene set enrichment analysis (ssGSEA) on the RNA expression data for the subject.

In some embodiments, determining the initial BC gene group scores comprises performing single-sample gene set enrichment analysis (ssGSEA) using the RNA expression levels for the at least three genes of the lymphoma cell pro-survival group listed in Table 2.

In some embodiments, the method further comprises determining that each normalized blood cancer gene group score is (i) below a first threshold value, (ii) between the first threshold value and a second threshold value, or (iii) above the second threshold value, wherein each of the first and second threshold values is determined using the RNA expression data for the cohort of subjects, and identifying each respective normalized BC gene group score as “low” when the respective normalized BC gene group score is below the first threshold value, identifying each respective normalized BC gene group score as “medium” when the respective gene group score is between the first threshold value and the second threshold value, or identifying each respective normalized BC gene group score as “high” when the respective gene group score is above the second threshold value.

In some embodiments, the method further comprises generating a visualization of the cytokine signature, the generating comprising generating a graphical user interface (GUI) having a plurality of GUI elements, each of the GUI elements representing a respective normalized BC gene group score part of the cytokine signature. In some embodiments, a particular GUI element of the plurality GUI elements represents the respective normalized BC cancer gene group score via a visual characteristic of the GUI element. In some embodiments, the visual characteristic is selected from the group consisting of a color, size, and font.

In some embodiments, the method further comprises identifying at least one therapeutic agent for administration to the subject using the cytokine signature.

In some embodiments, the method further comprises administering the at least one identified therapeutic agent to the subject.

In some embodiments, the at least one therapeutic agent is a cytokine therapy selected from an immune checkpoint inhibitor (ICI) or a tyrosine kinase inhibitor (TKI).

In some aspects, the disclosure provides a system comprising at least one computer hardware processor; at least one non-transitory computer-readable storage medium storing processor executable instructions that, when executed by the at least one computer hardware processor, cause the at least one computer hardware processor to perform a method as described herein.

In some aspects, the disclosure provides at least one non-transitory computer-readable storage medium storing processor executable instructions that, when executed by the at least one computer hardware processor, cause the at least one computer hardware processor to perform a method as described herein.

### BRIEF DESCRIPTION OF DRAWINGS

FIG. 1 provides an example of a processes for identifying the solid tumor cancer (STC) cytokine signature of a subject, according to some aspects of the invention. In some embodiments, the process includes obtaining a biopsy sample of a subject, extracting nucleic acids from the sample, sequencing the nucleic acids, and analyzing the nucleic acid sequences to identify a STC cytokine for the subject based on the gene expression data.

FIG. 2 provides an example of a processes for identifying the blood cancer (BC) cytokine signature of a subject, according to some aspects of the invention. In some embodiments, the process includes obtaining a biopsy sample of a subject, extracting nucleic acids from the sample, sequencing the nucleic acids, and analyzing the nucleic acid sequences to identify a BC cytokine for the subject based on the gene expression data.

FIG. 3 is a diagram depicting a flowchart of an illustrative process for processing sequencing data to obtain RNA expression data, according to some embodiments of the technology as described herein.

FIG. 4 is a diagram depicting an illustrative technique for determining gene group scores, according to some embodiments of the technology as described herein.

FIG. 5 shows a schematic illustrating using a cytokine signature to identify the tumor architecture of a subject, according to some aspects of the invention.

FIG. 6 shows a representative example of a cytokine signature visualization. The amplitude of each ray in the diagram represents a normalized gene group score.

FIGS. 7A-7D show representative validation of solid tumor gene groups, according to some aspects of the invention. FIG. 7A shows single gene expression analysis of genes in each gene group; top to bottom: CTL and Th1 activation group, angiogenesis inhibition group, B cell activation group, lymphocyte recruitment group, neutrophil recruitment and activation group; eosinophil recruitment group, Treg polarization group, metastasis induction group, CAF recruitment group; data on y-axis shown as log₂(TPM+1). FIG. 7B shows clustered heatmaps showing Spearman correlation coefficient between genes in each gene group. FIG. 7C shows heatmaps showing a Spearman correlation coefficient between the results of cell deconvolution analysis and single-sample gene set enrichment analysis (ssGSEA) scores for representative gene groups. Analysis of FIGS. 7A-7C was performed on a meta-cohort including all solid The Cancer Genome Atlas (TCGA) cancers except brain tumors. FIG. 7D shows boxplots showing how gene group score intensity differentiates a corresponding group of interest; y-axis is ssGSEA unscaled score.

FIGS. 8A-8C shows validation of selected gene group scores relating to lymphomas, according to some aspects of the invention. FIG. 8A shows clustered heatmaps showing Spearman correlation coefficients between genes in selected gene groups. FIG. 8B shows single gene expression analysis in selected gene groups. FIG. 8C shows heatmaps showing Spearman correlation coefficient between the results of cell deconvolution analysis and gene group scores in two Diffuse Large B-Cell Lymphoma (DLBCL) cohorts (TCGA and Nation Cancer Institute Center for Cancer Research database (NCICCR)). For FIGS. 8A-8C, analysis was performed on a meta-cohort for DLBCL (TCGA+NCICCR) and on a subset of MALY-DE (International Cancer Genome Consortium ((ICGC)) cohort for follicular lymphoma (FL).

FIGS. 9A-9B show unscaled ssGSEA scores of representative gene groups in healthy blood and lymph node samples, according to some aspects of the invention. *p<0.05, **p<0.01, ***p<0.001. FIG. 9A shows differences of gene group scores between healthy blood and normal lymph nodes. FIG. 9B shows differences of gene group scores between malignant and healthy lymph nodes.

FIGS. 10A-10B show unsealed ssGSEA scores of representative gene groups, according to some aspects of the invention. FIG. 10A shows differences of gene group scores between normal tissues from TCGA cohorts. FIG. 10B shows differences of gene group scores between normal and tumor tissues in dependence on the tissue of origin. *p<0.05, **p<0.01, ***p<0.001 FIG. 11 shows correlation analysis of cytokine signatures developed for solid tumors, according to some aspects of the invention. A clustered heatmap showing Spearman correlation coefficients for the gene group scores of cytokine signatures were counted for the pan-solid cohort which includes all solid cancers from the TCGA project, except brain tumors.

FIG. 12 shows solid tumor cancer (STC) cytokine signatures for colon adenocarcinoma (COAD) samples from the TCGA project, according to some aspects of the invention. Each column represents one sample. Panel on top corresponds to the subtype annotation: consensus molecular subtype (CMS) 1 (immune, 14%), hypermutated, microsatellite unstable and with strong immune activation; CMS2 subtype (canonical, 37%), epithelium-enriched, marked with WNT and MYC signaling activation; CMS3 (metabolic, 13%), epithelial with evident metabolic dysregulation; and CMS4 (mesenchymal, 23%), with prominent transforming growth factor-O activation, stromal invasion and angiogenesis. The heatmap shows the scaled ssGSEA score of each of the listed gene groups.

FIG. 13 shows a Cox regression analysis plot and the correspondent table for gene group scores describing anti-tumor and pro-tumor processes, according to some aspects of the invention. Analysis was performed on the COAD-TCGA cohort.

FIG. 14 shows cytokine signatures on stomach adenocarcinoma (STAD) and healthy colon samples from TCGA project, according to some aspects of the invention. Each column represents one sample. Panel on top corresponds to the tumor Molecular Functional Portrait (MFP) subtype annotation. Heatmap shows the scaled ssGSEA score of each of the listed gene groups.

FIGS. 15A-15B show representative data for performance of blood cancer (BC) cytokine signatures in a DLBCL cohort, according to some aspects of the invention. FIG. 15A shows a clustered heatmap showing Spearman correlation coefficients for gene group scores, counted for the DLBCL meta-cohort (TCGA+NCICCR). FIG. 15B shows a heatmap showing the scaled ssGSEA score of each of the listed gene groups in DLBCL Molecular Functional Portrait (MFP) subtypes. Each column represents one sample. Panel on top corresponds to the MFP subtype annotation.

FIG. 16 shows representative scores of solid tumor cancer (STC) gene groups in TCGA samples typed according to histological mapping of immune cell infiltration and dissemination, according to some aspects of the invention. Median values of scaled ssGSEA scores are shown in each column. Brisk Band-like—tumor infiltrating cells (TILs) are abundant, but are gathered around fibrotic capsule and poorly interact with tumor cells; Brisk Diffuse—TILs are abundant and distributed diffusely; Non-Brisk, Focal—few TILs, one or two aggregation spots; Non-Brisk, Multifocal—few TILs, but aggregated in multiple spots.

FIG. 17 shows that cytokine signatures can differentiate different patients, according to some aspects of the invention. The top panel shows heatmaps depicting cytokine signature scores in four TCGA-lung adenocarcinoma (LUAD) samples belonging to different tumor microenvironment (TME) subtypes: Immune-enriched, fibrotic; Immune-enriched, non-fibrotic; Fibrotic; Desert. The bottom panel shows bar plots of the cell type percentages per sample, calculated by internal cell deconvolution method.

FIG. 18 shows a representative visualization of a STC cytokine signature in a cutaneous melanoma sample.

FIGS. 19A-19C show cytokine signature visualizations for three patients that have a immune-enriched, Non-Fibrotic MFP tumor micro-environment type (TME). The tumor cytokine expression in FIG. 19A shows moderate effector cell activation, a high level of suppressive factors, and high Th2 response. The tumor cytokine expression in FIG. 19B shows a high level of effector-cell-activating signaling, high neutrophil/angiogenesis activation, and low suppressive factors. The tumor cytokine expression in FIG. 19C shows the highest level of effector-cell-activating signaling (of FIGS. 19A-19C) and high granulocyte activation.

FIGS. 20A-20B show representative data for Cox regression analysis for cytokine signatures and therapeutic response prediction. FIG. 20A shows high scores of TLS formation, Angiogenesis induction, CTL and Th1 activation, and Tumor growth arrest signatures were associated with significantly worse progression-free survival (PFS) and, thus, worse response to this kind of therapy, in a Clear Cell Renal Cell Carcinoma cohort treated with tyrosine kinase inhibitors (TKI); high scores of Angiogenesis inhibition, CTL exclusion, Pro-inflammatory cytokines, Eosinophils/Basophil activation and Metastasis inhibition signatures were favorable factors of longer PFS and, thus, better response to TKI. FIG. 20B shows high Macrophage and DC recruitment signature score was associated with significantly better survival in patients with Cutaneous Melanoma, Gastric Cancer and Head and Neck Squamous Carcinoma treated with anti-PD-1 immune checkpoint inhibitor (ICI); TLS formation, Angiogenesis inhibition, Tumor growth arrest, Angiogenesis induction, Stromal suppressive factors, Metastasis promotion and M2 polarization signature scores showed a tendency to be negative factors of overall survival.

FIG. 21 depicts an illustrative implementation of a computer system that may be used in connection with some embodiments of the technology described herein.

### DETAILED DESCRIPTION

Aspects of the disclosure relate to methods systems, and computer-readable storage media, which are useful for characterizing subjects having certain cancers, for example solid tumor cancers (STCs) and blood cancers (BCs, also referred to as hematological cancers). The disclosure is based, in part, on methods for identifying the cytokine signature of a subject having cancer by using gene expression data obtained from the subject. The inventors have surprisingly discovered that using methods described herein to characterize cytokine activity and immune processes in patients allows for more accurate patient stratification and prognosis relative to previously described patient characterization methods. In some embodiments, cytokine signatures described herein may be used to identify one or more therapeutic agents that can be administered to the subject.

Cytokines are small (e.g., ˜5 kDa to ˜25 kDa), secreted proteins that mediate a variety of effects on the immune system. For example, cytokines may elicit an inflammatory response, or induce production of immunomodulatory molecules and cells. In the context of cancer, cytokines also perform dual roles, both inhibiting tumor development and progression, as well as promoting growth, attenuating apoptosis, and facilitating invasion and metastasis of cancerous cells (e.g., as described by Dranoff (2004) Nature Reviews Cancer 4, 11-22). Although the importance of understanding cytokine activity in the tumor microenvironment has been recognized, currently available methods of cytokine profiling face several challenges. For example, the pleiotropic effects of different cytokines, and the pleiotropic effects of the same cytokine in different conditions (e.g., normal tissue vs. tumor tissue) are difficult to resolve, there remains a lack of standardization for clinical cytokine measurement techniques, for example as described by Liu et al. (2021) Adv Sci (Weinh). 2021 August; 8(15):e200443. Moreover, cytokines often have more than one specific receptor, and cytokines from one family may induce signal transduction pathways through cognate receptors from other families.

Aspects of the disclosure relate to statistical techniques for analyzing expression data (e.g., RNA expression data), which was obtained from a biological sample obtained from a subject that has cancer, is suspected of having cancer, or is at risk of developing cancer, in order to generate a cytokine signature for the subject and use this signature to identify a particular prognosis that the subject may have or therapy to which the subject is likely to respond.

The inventors have recognized that analyzing RNA expression for groups of genes in biological pathways relating to cytokine expression and/or function (e.g., gene groups set forth in Tables 1 and 2) allows for generation of cytokine signatures that are indicative of the subject's immunological activity and likely response to certain therapeutics (e.g., immune checkpoint inhibitors (ICIs) and tyrosine kinase inhibitors (TKIs)).

The use of cytokine signatures comprising the combinations of gene group scores described by the disclosure represents an improvement over previously described cytokine profiling because the specific groups of genes used to produce the cytokine signatures described herein better reflect the immunological status of a subject's tumor microenvironment (TME) because these gene groups are associated with the underlying biological pathways controlling cancer cell behavior and the host immune response to cancer cells. These focused combinations of gene groups (e.g., gene groups consisting of some or all of the gene group genes listed in Table 1 or Table 2) are unconventional, and differ from previously described molecular signatures, which do not account for the high levels of genotypic and phenotypic heterogeneity within different cancer types.

The cytokine signature generation methods described herein have several utilities. For example, identifying a subject's cytokine signature using methods described herein may allow for the subject to be diagnosed as having (or being at a high risk of developing) forms of cancer that are unlikely (or likely) to respond to a particular type of therapy (e.g., an ICI or TKI). For example, the inventors have recognized that subjects having certain solid tumor cancers (e.g., clear cell renal carcinoma, ccRCC) that are characterized as having a cytokine signature comprising a “high” normalized gene group scores for TLS formation, Angiogenesis induction, CTL and Th1 activation, and Tumor growth arrest are less likely to respond to treatment with a TKI than subjects having a cytokine signature comprising “high” normalized gene group scores for angiogenesis inhibition, CTL exclusion, pro-inflammatory cytokines, eosinophils/basophil activation, or metastasis inhibition. In another example, the inventors recognized that subjects having cutaneous melanoma, gastric cancer, or head and neck squamous carcinoma that are characterized as having a cytokine signature comprising “high” gene group scores for macrophage and DC recruitment gene groups are more likely to respond to ICI treatment (e.g., anti-PD-1 therapy) than subjects having a cytokine signature comprising “high” gene group scores for TLS formation, angiogenesis inhibition, tumor growth arrest, angiogenesis induction, stromal suppressive factors, metastasis promotion, or M2 polarization.

Aspects of the disclosure relate to identifying the cytokine signature of a subject. As used herein, the term “subject” means any mammal, including mice, rabbits, and humans. In one embodiment, the subject is a human or non-human primate. The terms “individual” or “subject” may be used interchangeably with “patient.” In some embodiments, the biological sample may be any sample from a subject known or suspected of having cancerous cells or pre-cancerous cells.

In some embodiments, a subject has, is suspected of having, or at risk of developing cancer. As used herein, “cancer” refers to any malignant and/or invasive growth or tumor caused by abnormal cell growth in a subject, including solid tumors, blood cancer, bone marrow or lymphoid cancer, etc. A subject “having cancer” exhibits one or more signs or symptoms of cancer, for example the presence of cancerous cells (e.g., tumor cells). In some embodiments, a subject having cancer has been diagnosed as having cancer by a clinician (e.g., physician) and/or has received a positive result of a laboratory test that indicates the subject as having cancer. A subject “suspected of having cancer” exhibits one or more signs or symptoms of cancer (e.g., presence of a tumor or tumor cells, fever, swelling, bleeding, etc.) but has not been diagnosed by a clinician as having cancer. A subject “at risk of having cancer” may or may not exhibit one or more signs or symptoms of cancer but may comprise one or more genetic mutations that increases the risk that the subject will develop cancer (e.g., relative to a normal healthy subject not having such mutations).

### Solid Tumor Cancers

Aspects of the disclosure relate to methods for generating a cytokine signature of a subject having a solid tumor cancer (STC). As used herein, a “solid tumor cancer” or “STC” refers to a cancer that forms a solid mass of tissue comprising cancer cells (e.g., a solid tumor or tumors). In some embodiments, a solid tumor cancer is a carcinoma. In some embodiments, a solid tumor cancer is a sarcoma. Examples of solid tumor cancers include but are not limited to bone cancers bladder cancers, breast cancers, cervical cancers, colon cancers, rectal cancers, endometrial cancers, kidney cancers, lip and oral cancers, stomach cancers, gastrointestinal cancers, liver cancers, melanomas, mesotheliomas, lung cancers, (e.g., non-small cell lung cancers), skin cancers (e.g., non-melanoma skin cancers), ovarian cancers, pancreatic cancers, prostate cancers, muscle cancers, thyroid cancers, head and neck cancers, brain cancers, etc.

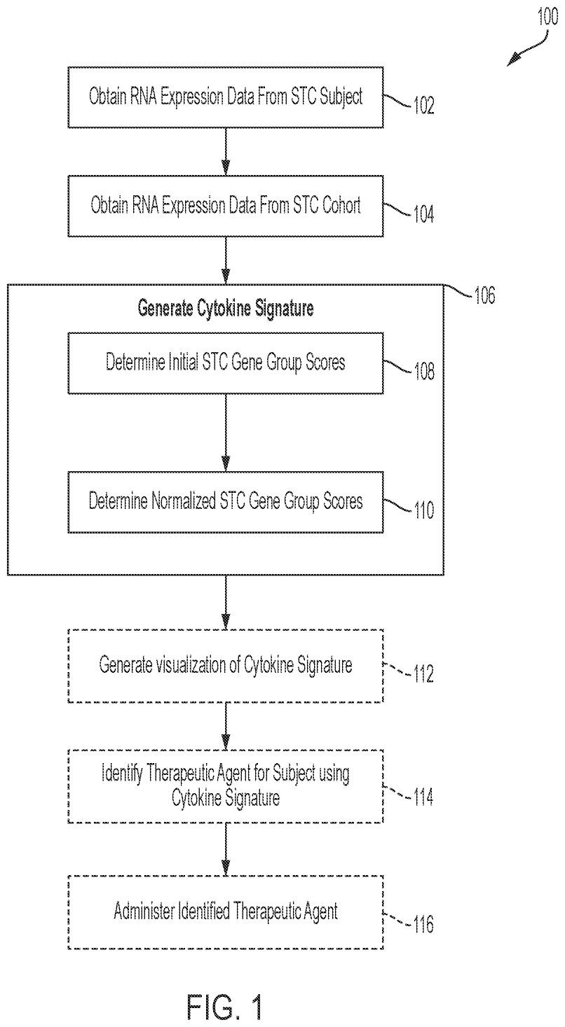

FIG. 1 is a flowchart of an illustrative process 100 for determining a solid tumor cancer (STC) cytokine signature for a subject, and, optionally, using the cytokine signature of the subject to identify whether or not the subject is likely to respond to a therapy, e.g., an immunotherapy, TKI, etc.

Various (e.g., some or all) acts of process 100 may be implemented using any suitable computing device(s). For example, in some embodiments, one or more acts of the illustrative process 100 may be implemented in a clinical or laboratory setting. For example, one or more acts of the process 100 may be implemented on a computing device that is located within the clinical or laboratory setting. In some embodiments, the computing device may directly obtain RNA expression data from a sequencing apparatus located within the clinical or laboratory setting. For example, a computing device included in the sequencing apparatus may directly obtain the RNA expression data from the sequencing apparatus. In some embodiments, the computing device may indirectly obtain RNA expression data from a sequencing apparatus that is located within or external to the clinical or laboratory setting. For example, a computing device that is located within the clinical or laboratory setting may obtain expression data via a communication network, such as Internet or any other suitable network, as aspects of the technology described herein are not limited to any particular communication network.

Additionally or alternatively, one or more acts of the illustrative process 100 may be implemented in a setting that is remote from a clinical or laboratory setting. For example, the one or more acts of process 100 may be implemented on a computing device that is located externally from a clinical or laboratory setting. In this case, the computing device may indirectly obtain RNA expression data that is generated using a sequencing apparatus located within or external to a clinical or laboratory setting. For example, the expression data may be provided to computing device via a communication network, such as Internet or any other suitable network.

It should be appreciated that, in some embodiments, not all acts of process 100, as illustrated in FIG. 1, may be implemented using one or more computing devices. For example, the act 116 of administering one or more therapeutic agents to the subject may be implemented manually (e.g., by a clinician).

Process 100 begins at act 102 where RNA expression data from a subject having an STC is obtained. In some embodiments, the RNA expression data may be obtained from sequencing data obtained by sequencing a biological sample (e.g., tissue biopsy and/or tumor tissue) obtained from the subject using any suitable sequencing technique. The sequencing data may include sequencing data of any suitable type, from any suitable source, and be in any suitable format. Examples of sequencing data, sources of sequencing data, and formats of sequencing data are described herein including in the section called “Obtaining RNA Expression Data.”

As one illustrative example, in some embodiments, the sequencing data may comprise bulk sequencing data. The bulk sequencing data may comprise at least 1 million reads, at least 5 million reads, at least 10 million reads, at least 20 million reads, at least 50 million reads, or at least 100 million reads. In some embodiments, the sequencing data comprises bulk RNA sequencing (RNA-seq) data, single cell RNA sequencing (scRNA-seq) data, or next generation sequencing (NGS) data. In some embodiments, the sequencing data comprises microarray data.

Next, process 100 proceeds to act 104, where RNA expression data from a cohort of patients having the same type of STC as the subject (e.g., a TCGA cohort) is obtained. For example, if the subject has a stomach adenocarcinoma (STAD), the RNA expression data may be obtained from a cohort of patients having STAD. In some embodiments, the RNA expression data may be obtained from sequencing data obtained by sequencing a plurality of biological samples (e.g., tissue biopsy and/or tumor tissue) obtained from a plurality of subjects using any suitable sequencing technique. The sequencing data may include sequencing data of any suitable type, from any suitable source, and be in any suitable format. Examples of sequencing data, sources of sequencing data, and formats of sequencing data are described herein including in the section called “Obtaining RNA Expression Data.”

As one illustrative example, in some embodiments, the sequencing data may comprise bulk sequencing data. The bulk sequencing data may comprise at least 1 million reads, at least 5 million reads, at least 10 million reads, at least 20 million reads, at least 50 million reads, or at least 100 million reads. In some embodiments, the sequencing data comprises bulk RNA sequencing (RNA-seq) data, single cell RNA sequencing (scRNA-seq) data, or next generation sequencing (NGS) data. In some embodiments, the sequencing data comprises microarray data.

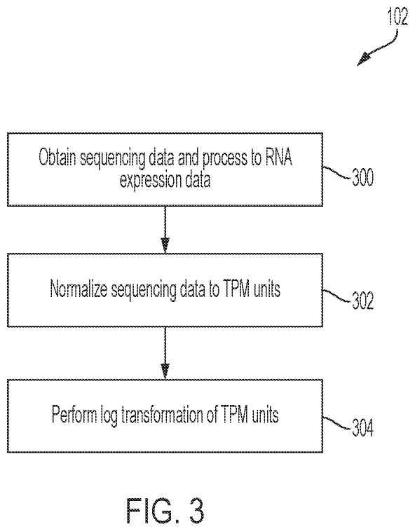

In some embodiments, the RNA expression data is obtained by processing sequencing data obtained from the subject or cohort. This may be done in any suitable way and may involve normalizing bulk sequencing data to transcripts-per-million (TPM) units (or other units) and/or log transforming the RNA expression levels in TPM units. Converting the data to TPM units and normalization are described herein including with reference to FIG. 3.

Next, process 100 proceeds to act 106, where a solid tumor cancer (STC) cytokine signature is generated for the subject using the RNA expression data obtained at act 102 (e.g., from bulk-sequencing data, converted to TPM units and subsequently log-normalized, as described herein including with reference to FIG. 3), and the RNA expression data obtained at act 104 (e.g., from bulk-sequencing data, converted to TPM units and subsequently log-normalized, as described herein including with reference to FIG. 3).

As described herein, in some embodiments, an STC cytokine signature comprises two or more (e.g., 2, 3, 4, 5, 6, 7, 8, 9, 10, 11, 12, 13, 14, 15, 16, 17, 18, 19, 20, 21, 22, 23, 24, etc.) gene group scores. In some embodiments, the two or more gene group scores comprise gene group scores (which may also be referred to as gene group enrichment scores or gene group expression scores) for some or all of the gene groups shown in Table 1.

Accordingly, act 106 comprises: act 108 where the initial STC gene group scores are determined, and act 110 where the initial STC gene group scores determined at act 108 are normalized using RNA expression data for the cohort of subjects.

In some embodiments, determining the initial STC gene group scores comprises determining, for each of multiple (e.g., some or all of the) gene groups listed in Table 1, a respective gene group score. In some embodiments, determining the gene group scores comprises determining respective gene group scores for 2, 3, 4, 5, 6, 7, 8, 9, 10, 11, 12, 13, 14, 15, 16, 17, 18, 19, 20, 21, 22, 23, or 24 gene groups (e.g., gene groups listed in Table 1). The gene group score for a particular gene group may be determined using RNA expression levels for at least some of the genes in the gene group (e.g., the RNA expression levels obtained at act 102). The RNA expression levels may be processed using a gene set enrichment analysis (GSEA) technique to determine the score for the particular gene group.

In some embodiments, determining the initial STC gene group scores comprises determining gene group scores for one or more gene groups associated with pro-tumor effects. In some embodiments, the one or more gene groups associated with pro-tumor effects are selected from among the myeloid inflammation groups, the Type 2 response groups, the immune suppression groups, the tumor promotion groups, and the stroma activation groups, listed in Table 1. In some embodiments, determining the STC gene group scores comprises determining gene group scores from one, some, or all of the myeloid inflammation groups (e.g., 0, 1 or 2 gene groups of the myeloid inflammation gene groups in Table 1), the Type 2 response groups (e.g., 0, 1, 2, 3, or 4 gene groups of the Type 2 response groups in Table 1), the immune suppression groups (e.g., 0, 1, 2, 3, or 4 gene groups of the immune suppression gene groups in Table 1), the tumor promotion groups (e.g., 0, 1, 2, or 3 of the tumor suppression groups in Table 1), and/or the stroma activation groups (e.g., 0, 1, or 2 of the stromal activation groups in Table 1), listed in Table 1.

In some embodiments, determining the initial STC gene group scores comprises determining STC gene group scores for one or more gene groups associated with anti-tumor effects. In some embodiments, the one or more gene groups associated with anti-tumor effects are selected from Type 1 response groups and tumor suppression groups listed in Table 1. In some embodiments, determining the STC gene group scores comprises determining gene group scores from one, some, or all of the Type 1 response groups (e.g., 0, 1, 2, or 3 gene groups of the Type 1 response groups in Table 1) and/or tumor suppression groups (e.g., 0, 1, 2, or 3 gene groups of the tumor suppression groups in Table 1) listed in Table 1.

In some embodiments, determining the initial STC gene group scores comprises determining STC gene group scores for one or more gene groups associated with B cell effects. In some embodiments, the one or more gene groups associated with B cell effects are selected from B cell function groups and immune cell recruitment groups. In some embodiments, determining the STC gene group scores comprises determining gene group scores from one, some, or all of the B cell function groups (e.g., 0 or 1 gene groups of the B cell function groups in Table 1) and/or the immune cell recruitment groups (e.g., 0, 1, or 2 gene groups of the immune cell recruitment groups in Table 1) in Table 1.

For example, in some embodiments, determining the initial STC gene group scores comprises: determining gene group scores using the RNA expression levels for at least three genes from each of at least two of the gene groups, the gene groups including Type 1 response groups (e.g., CTL and Th1 cells activation group, M1 polarization group, TLS formation group), tumor suppression groups (e.g., tumor growth arrest group, metastasis inhibition group, angiogenesis inhibition group), B cell function groups (e.g., B cell activation group), immune cell recruitment groups (e.g., lymphocyte recruitment group, macrophage and DC recruitment group), myeloid inflammation groups (e.g., pro-inflammatory cytokines group, neutrophil recruitment and activation group), Type 2 response groups (e.g., Th2 response group, M2 polarization group, eosinophil/basophil recruitment group, eosinophil/basophil activation group), immune suppression groups (e.g., stromal suppressive factors group, myeloid suppressive factors group, CTL exclusion group, Treg polarization group), tumor promoting groups (e.g., tumor growth promotion group, induction of EMT group, metastasis promotion group), and/or stromal activation groups (e.g., angiogenesis induction group, CAF recruitment group)

In some embodiments, determining the initial STC gene group scores comprises: determining gene group scores using the RNA expression levels for all genes in each of the following gene groups: Type 1 response groups (e.g., CTL and Th1 cells activation group, M1 polarization group, TLS formation group), tumor suppression groups (e.g., tumor growth arrest group, metastasis inhibition group, angiogenesis inhibition group), B cell function groups (e.g., B cell activation group), immune cell recruitment groups (e.g., lymphocyte recruitment group, macrophage and DC recruitment group), myeloid inflammation groups (e.g., pro-inflammatory cytokines group, neutrophil recruitment and activation group), Type 2 response groups (e.g., Th2 response group, M2 polarization group, eosinophil/basophil recruitment group, eosinophil/basophil activation group), immune suppression groups (e.g., stromal suppressive factors group, myeloid suppressive factors group, CTL exclusion group, Treg polarization group), tumor promoting groups (e.g., tumor growth promotion group, induction of EMT group, metastasis promotion group), and/or stromal activation groups (e.g., angiogenesis induction group, CAF recruitment group).

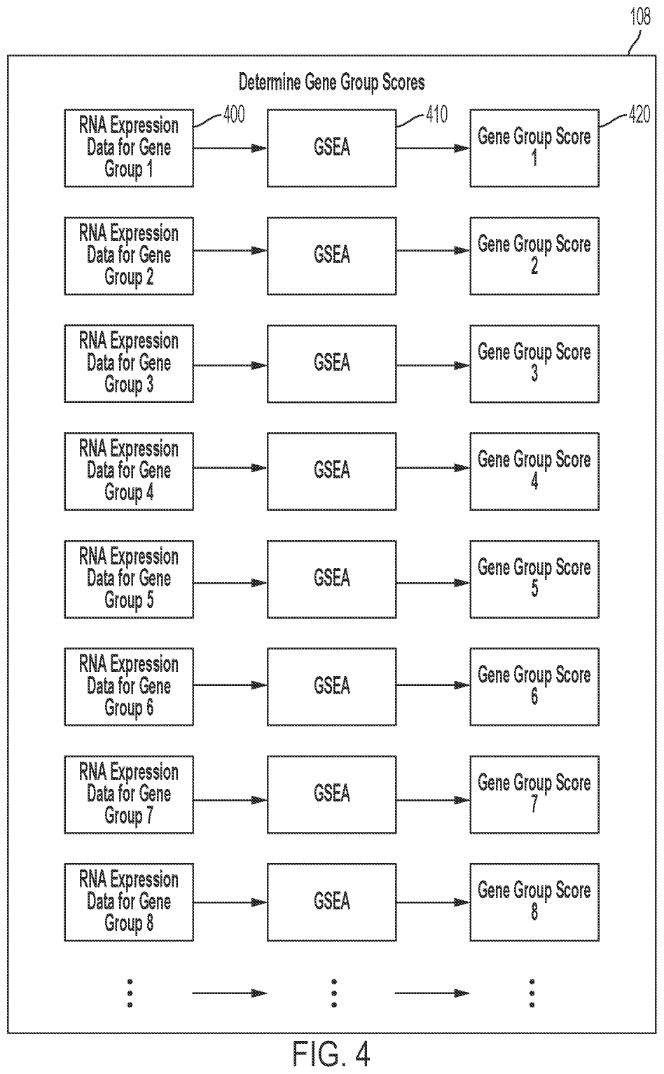

Aspects of determining the gene group scores are described herein, including with reference to FIG. 4 and in the Section titled “Cytokine Signatures”.

As described above, at act 110, the normalized STC gene group score is determined. In some embodiments, the normalized gene STC gene group score may be determined by normalizing the initial STC gene group scores relative to corresponding gene group scores generated using RNA expression data from a reference cohort of patients with the same STC type as the subject. This may be done in any suitable way. For example, the reference cohort may have N patients and normalizing a score for a particular gene group for a patient P (not in the reference cohort) may involve: (1) determining a gene group score for the same particular gene group for each of the N patients in the reference cohort to obtain a set of gene group scores for that reference cohort; (2) identifying the smallest and largest gene group score in the set of gene group scores for that reference cohort; (3) considering the smallest gene group score as 0% and the largest gene group score as 100% and dividing the range therebetween uniformly into percentages (e.g., if the smallest score is 120 and the largest score is 320, then 120-121 would correspond to 0%, 122-123 would correspond to 1%, 124-125 would correspond to 2%, and so on); and (4) determining the percentage in the range of scores for the reference cohort that corresponds to the score for the particular gene group (e.g., a score of 124.2 for patient P would map to 2% indicating that relative to the reference cohort, patient P's score is in the bottom 2%). It should be appreciated that the normalization may be done in other ways: using quantiles of the set of gene group scores for the reference cohort rather than a uniform division of the range, by computing a discrete cumulative distribution function (CDF) from the reference cohort scores and using an inverse of the CDF to identify a number between 0 and 1 for the score of the particular gene group, and/or in any other suitable way. In this way, a gene group score for a particular gene group can be turned into a normalized gene group score that represents a percentage relative to the reference cohort, which provides information about how large the magnitude of the gene group score for a particular patient is relative to the range of the same gene group scores seen for a reference cohort.

In some embodiments, the RNA expression data for the cohort may obtained from the TCGA project. However, the skilled artisan will recognize that other suitable sequencing datasets and/or RNA expression datasets from sources other than TCGA may be used to obtain RNA expression data for the cohort (so long as the cohort used has the same type of STC as the subject). In some embodiments, the normalized gene group scores of the subject may be assigned a “High”, “Medium”, or “Low” gene group score designation, according to thresholds set using the gene group scores of the cohort used for normalization. For example, if the normalized gene group score of a subject is less than a first threshold, the normalized gene group score may be identified as being “Low”. If the normalized gene group score of a subject is more than a first threshold and less than a second threshold, the normalized gene group score may be identified as being “Medium” (or “Med”). If the normalized gene group score of a subject normalized gene group score of a subject is more than the second threshold, the normalized gene group score may be identified as being “High”. In some embodiments, a normalized STC gene group score less than 17% is identified as being “Low”, more than 83% is identified as being “High”, and between 17%-83% is identified as being “Medium”.

Optionally, process 100 proceeds to act 112, where a visualization of the subject's cytokine signature may be generated. In some embodiments, generating the visualization of the cytokine signature comprises generating a graphical user interface (GUI) having a plurality of GUI elements, each of the GUI elements representing a respective normalized STC gene group score part of the cytokine signature. In some embodiments, a particular GUI element of the plurality GUI elements represents the respective normalized STC gene group score via a visual characteristic of the GUI element. In some embodiments, the visual characteristic is selected from the group consisting of a color, size, and font. In some embodiments, the GUI may be interactive in that a user can indicate a selection of one or more of the GUI elements (e.g., a GUI element representing a particular gene group) and receive more information in response to that selection (e.g., information indicating the normalized gene scores for one or more (e.g., all) genes in the particular gene group).

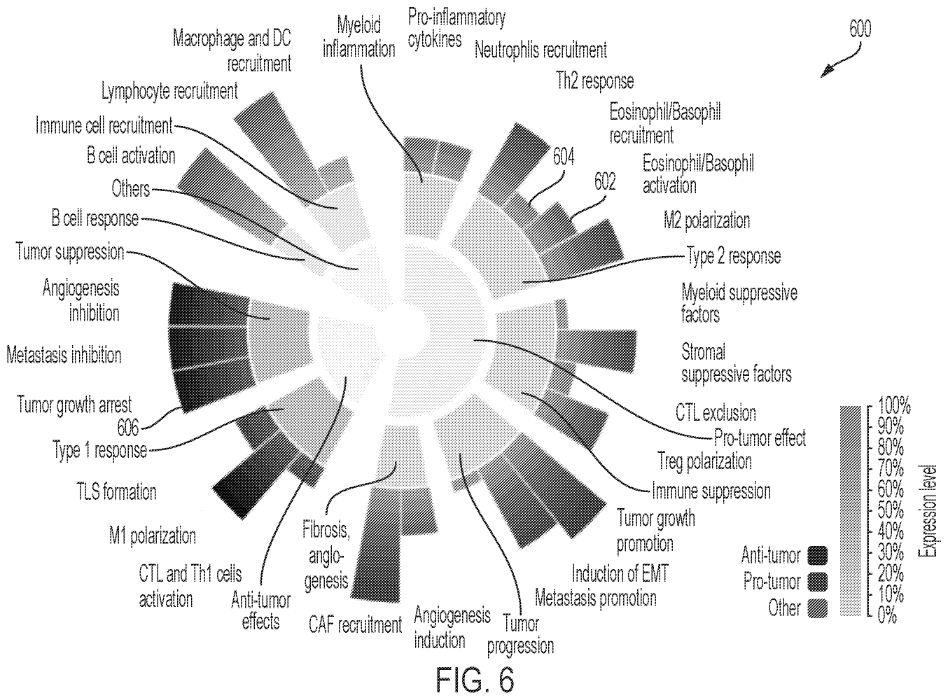

For example, in some embodiments, the visualization of the cytokine signature may be a GUI showing a “solar” diagram. The diagram may be used to illustrate the intensity of different cytokine-mediated biological processes represented by the different gene groups. The solar diagram provides a visual representation of the normalized gene group scores for the gene groups. In some embodiments, the solar diagram represents the normalized gene group scores by respective ray so that each ray corresponds to a respective gene group and a visual characteristic of the ray depends on the normalized gene group score for that gene group. Each normalized gene group score may take on a value representing a percentage between 0 and 100% (or, e.g., a number between 0 and 1 or any other suitable scale to represent a percentage). For example, as shown in FIG. 6, the height of the rays represents the normalized gene group scores of the gene groups represented by those rays. For instance, the heights of rays 602, 604, and 606 represent the normalized gene group scores of the Eosinophil/Basophil activation gene group, the Eosinophil/Basophil recruitment gene group, and the Tumor growth arrest group, respectively. As can be seen in this visualization, the normalized gene group score of the Eosinophil/Basophil activation gene group (represented by ray 602) is greater than the normalized gene group score of the Eosinophil/Basophil recruitment gene group (represented by ray 604).

As can also be seen in FIG. 6, the solar diagram includes concentric rings indicative of a hierarchical grouping of the gene groups. The inner-most ring, for example, groups all the outer-most gene groups into three “high-level” sets of groups: “Anti-tumor effects”, “Pro-tumor effects” and “Others”. The second ring, for example, groups all the outer-most gene groups into 9 “mid-level” sets of groups: “Tumor suppression”, “Type 1 response”, “Fibrosis, angiogenesis”, “Tumor progression”, “Immune suppression”, “Type 2 response”, “Myeloid inflammation”, “Immune cell recruitment”, and “B cell response”. The constituent gene groups of these mid-level sets of groups are shown in the next ring.

As can also be seen in FIG. 6, the color, shading, or other characteristics can be used to indicate whether a gene group or a set of gene groups is pro-tumor or anti-tumor.

In some embodiments, process 100 proceeds to act 114, where the subject's likelihood of responding to a therapy is identified using the cytokine signature identified at act 106. In some embodiments, when the subject has been identified as having a “high” normalized STC gene group score for one or more of the following gene groups: immune cell recruitment groups, B cell response groups, or Type 1 response groups, the subject is identified as being a candidate for treatment with an immune checkpoint inhibitor (ICI). In some embodiments, when the subject is identified as having a “low normalized STC gene group score for one or more of the following gene groups: tumor suppression gene groups, immune suppression gene groups, or stromal activation gene groups, the subject is identified as being a candidate for treatment with an immune checkpoint inhibitor (ICI). In some embodiments, the ICI is an anti-PD1 therapy. In some embodiments, when the subject has been identified as having a “high” normalized STC gene group score for one or more of the following gene groups: myeloid inflammation gene groups, or tumor suppression gene groups, the subject is identified as being a candidate for treatment with a tyrosine kinase inhibitor (TKI). In some embodiments, when the subject is identified as having a “low normalized STC gene group score for one or more of the following gene groups: Type 1 response gene groups or stromal activation groups, the subject is identified as being a candidate for treatment with a TKI. Aspects of identifying whether or not a subject is likely to respond to a therapy are described herein including in the section below titled “Therapeutic Indications.”

In some embodiments, process 100 completes after act 114 completes. In some such embodiments, the determined cytokine signature (or visualization of cytokine signature generated at act 112), and/or the identified likelihood the subject will respond to a therapy may be stored for subsequent use, provided to one or more recipients (e.g., a clinician, a researcher, etc.).

However, in some embodiments, one or more other acts are performed after act 114. For example, in the illustrated embodiment of FIG. 1, process 100 may include one or more of optional acts 112, 114, and 116 shown using dashed lines in FIG. 1. For example, when a subject is identified as having an increased likelihood of responding to immunotherapy at act 114, the subject is administered one or more immunotherapies at act 116. Examples of immunotherapies and other therapies are provided herein.

It should be appreciated that although acts 112, 114, and 116 are indicated as optional in the example of FIG. 1, in other embodiments, one or more other acts may be optional (in addition to or instead of acts 112, 114, and 116). For example, in some embodiments, acts 102 and 104 may be optional (e.g., when the RNA expression data is obtained previously, process 100 may begin at act 106 by accessing the previously obtained RNA expression data). In some embodiments, the process 100 may comprise acts 102, 104, 106, 108, 110, 112 and 114, without act 116. In some embodiments, the process 100 may comprise acts 102, 104, 106, 108, 110, 114, and 116, without act 112.

TABLE 1
List of gene groups for solid tumor cancer (STC) cytokine signatures

Genes

Anti-tumor effects groups
Type 1 response groups

| | |
|---|---|
| CTL and Th1 cells | IFNG, IL18, IL15RA, TNF, IL27, CCL5, TNFSF11, CD40LG, |
| activation group | FLT3LG, TNFSF9, CD70 |
| M1 polarization group | CCL21, IFNG, CSF2, TNF, IL23A, IL12B, IL27 |
| TLS formation group | CXCL11, CCL18, CXCL10, CXCL9, CCL2, CXCL13, CCL8, CCL5, |
| | CCL4, CCL3, CCL19, CCL21 |

Tumor suppression groups

| | |
|---|---|
| Tumor growth arrest | IFNG, CXCR2, ACKR2, ACKR4, ACKR1, BAMBI, TNFSF10, LTA |
| group | |
| Metastasis inhibition | ACKR1, ACKR2, ACKR4, CCL28 |
| group | |
| Angiogenesis inhibition | IFNG, IL12A, IL12B, CXCL9, CXCL10, CXCL11, CXCR3, CCL5, |
| group | CCR5, ACKR1, ARRB2, TNFSF15 |

B cell effects groups
B cell function groups

| | |
|---|---|
| B cell activation group | IFNG, IL4, IL10, TNFSF13B TNFSF13 |

Immune cell recruitment groups

| | |
|---|---|
| Lymphocyte | CXCL9, CXCL10, CXCL11, CCL5, CXCL13, CCL20, CCL21, |
| recruitment group | CCL19, CCL17, CCL22 |
| Macrophage and DC | CCL2, CCL3, TGFB1, CSF1, XCL1, CXCL12, CCL18 |
| recruitment group | |

Pro-tumor effects groups
Myeloid inflammation groups

| | |
|---|---|
| Pro-inflammatory | IL1A, IL1B, TNF, IL6, IL23A, LIF, CCL2, CCL3, CCL4, CXCL2, |
| cytokines group | CXCL1 |
| Neutrophil recruitment | CXCL1, CXCL2, CXCL3, PF4, CXCL5, CXCL8, CSF2, CSF3, PPBP, |
| and activation group | CXCL6 |

Type 2 response groups

| | |
|---|---|
| Th2 response group | IL4, IL10, IL11, IL13, CCL8, TSLP, CCL13 |
| M2 polarization group | IL10, IL33, CCL18, CCL24, TGFB2, TGFB3 |
| Eosinophil/Basophil | CCL8, CCL11, CCL13, CCL24, CCL26 |
| recruitment group | |
| Eosinophil/Basophil | IL33, ENPP3, IL1RL1, IL5RA, ANGPT1, IL13 |
| activation group | |

Immune suppression groups

| | |
|---|---|
| Stromal suppressive | TGFB2, TGFB3, TDO2, TGFBI, IL6, IL11, TSLP |
| factors group | |
| Myeloid suppressive | IL10, FGL2, IDO1, EBI3, IL4I1, TGFB1, TGFBI |
| factors group | |
| CTL exclusion group | CXCL12, EDNRB, PDGFC, FGF2, GAS6, TNFAIP6, TGFBI, |
| | VEGFA, AXL |
| Treg polarization group | TGFB1, TGFB2, TGFB3, IL2RA, IL10, IDO1, CCL17, CCL22 |

Tumor promotion groups

| | |
|---|---|
| Tumor growth | IL6, HGF, TGFB3, FGF7, FGF10, IGF1, EGF |
| promotion group | |
| Induction of EMT | IL6, LIF, CCL7, CCL8, CXCL8, TNFSF11, ARTN |
| group | |
| Metastasis promotion | OSM, IL1B, CCR4, CCR6, CXCR4, HGF, CXCL8, TNFSF11, |
| group | TGFB1, IL15RA, HIF1A |

Stroma activation groups

| | |
|---|---|
| Angiogenesis induction | VEGFA, VEGFC, FGF2, PDGFA, PDGFB, PDGFC, TGFB1, |
| group | TNFSF12 |
| CAF recruitment group | FGF1, FGF2, TGFB1, TGFB2, TGFB3, CXCL12, IL6, MMP2 |

### Blood Cancers

Aspects of the disclosure relate to methods for generating a cytokine signature of a subject having a blood cancer (BC). Blood cancers may also be referred to as “liquid cancers” or “hematological cancers”. As used herein, a “blood cancer” or “BC” refers to a cancer that originates in blood or lymph cells and is detectable in the blood or other bodily fluids (e.g., lymph fluid) of a subject. In some embodiments, a blood cancer is a lymphoma. In some embodiments, a blood cancer is a leukemia. In some embodiments, a blood cancer is a myeloma. Examples of blood cancers include but are not limited to Hodgkin lymphoma, B lymphoblastic leukemia, T lymphoblastic leukemia, B lymphoblastic lymphoma, T lymphoblastic lymphoma, diffuse large B cell lymphoma (DLBCL), nervous system lymphoma, Burkitt lymphoma, mantle cell lymphoma, hairy cell leukemia, Waldenstrom's, B cell lymphoma, multiple myeloma (MM), acute myeloid leukemia (AML), etc.

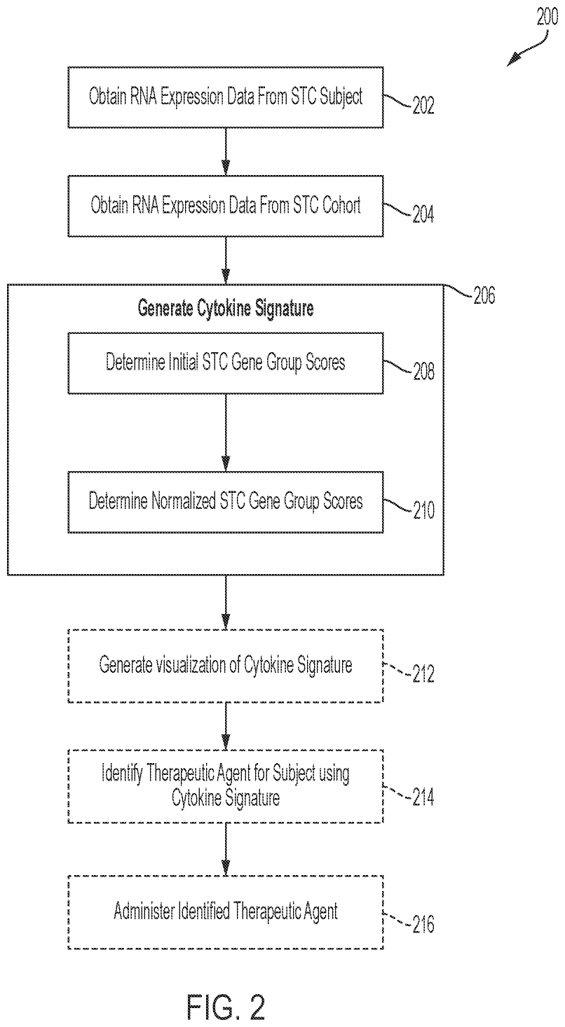

FIG. 2 is a flowchart of an illustrative process 200 for determining a blood cancer (BC) cytokine signature for a subject, and, optionally, using the cytokine signature of the subject to identify whether or not the subject is likely to respond to a therapy, e.g., an immunotherapy, TKI, etc.

Various (e.g., some or all) acts of process 200 may be implemented using any suitable computing device(s). For example, in some embodiments, one or more acts of the illustrative process 200 may be implemented in a clinical or laboratory setting. For example, one or more acts of the process 200 may be implemented on a computing device that is located within the clinical or laboratory setting. In some embodiments, the computing device may directly obtain RNA expression data from a sequencing apparatus located within the clinical or laboratory setting. For example, a computing device included in the sequencing apparatus may directly obtain the RNA expression data from the sequencing apparatus. In some embodiments, the computing device may indirectly obtain RNA expression data from a sequencing apparatus that is located within or external to the clinical or laboratory setting. For example, a computing device that is located within the clinical or laboratory setting may obtain expression data via a communication network, such as Internet or any other suitable network, as aspects of the technology described herein are not limited to any particular communication network.

Additionally or alternatively, one or more acts of the illustrative process 200 may be implemented in a setting that is remote from a clinical or laboratory setting. For example, the one or more acts of process 200 may be implemented on a computing device that is located externally from a clinical or laboratory setting. In this case, the computing device may indirectly obtain RNA expression data that is generated using a sequencing apparatus located within or external to a clinical or laboratory setting. For example, the expression data may be provided to computing device via a communication network, such as Internet or any other suitable network.

It should be appreciated that, in some embodiments, not all acts of process 200, as illustrated in FIG. 2, may be implemented using one or more computing devices. For example, the act 216 of administering one or more therapeutic agents to the subject may be implemented manually (e.g., by a clinician).

Process 200 begins at act 202 where RNA expression data from a subject having a BC is obtained. In some embodiments, the RNA expression data may be obtained from sequencing data obtained by sequencing a biological sample (e.g., tissue biopsy and/or tumor tissue) obtained from the subject using any suitable sequencing technique. The sequencing data may include sequencing data of any suitable type, from any suitable source, and be in any suitable format. Examples of sequencing data, sources of sequencing data, and formats of sequencing data are described herein including in the section called “Obtaining RNA Expression Data.”

As one illustrative example, in some embodiments, the sequencing data may comprise bulk sequencing data. The bulk sequencing data may comprise at least 1 million reads, at least 5 million reads, at least 10 million reads, at least 20 million reads, at least 50 million reads, or at least 100 million reads. In some embodiments, the sequencing data comprises bulk RNA sequencing (RNA-seq) data, single cell RNA sequencing (scRNA-seq) data, or next generation sequencing (NGS) data. In some embodiments, the sequencing data comprises microarray data.

Next, process 200 proceeds to act 204, where RNA expression data from a cohort of patients having the same type of BC as the subject (e.g., a TCGA cohort) is obtained. For example, if the subject has a multiple myeloma (MM) the RNA expression data may be obtained from a cohort of patients having MM. In some embodiments, the RNA expression data may be obtained from sequencing data obtained by sequencing a plurality of biological samples (e.g., tissue biopsy and/or tumor tissue) obtained from a plurality of subjects using any suitable sequencing technique. The sequencing data may include sequencing data of any suitable type, from any suitable source, and be in any suitable format. Examples of sequencing data, sources of sequencing data, and formats of sequencing data are described herein including in the section called “Obtaining RNA Expression Data.”

As one illustrative example, in some embodiments, the sequencing data may comprise bulk sequencing data. The bulk sequencing data may comprise at least 1 million reads, at least 5 million reads, at least 10 million reads, at least 20 million reads, at least 50 million reads, or at least 100 million reads. In some embodiments, the sequencing data comprises bulk RNA sequencing (RNA-seq) data, single cell RNA sequencing (scRNA-seq) data, or next generation sequencing (NGS) data. In some embodiments, the sequencing data comprises microarray data.

In some embodiments, the RNA expression data is obtained by processing sequencing data obtained from the subject or cohort. This may be done in any suitable way and may involve normalizing bulk sequencing data to transcripts-per-million (TPM) units (or other units) and/or log transforming the RNA expression levels in TPM units. Converting the data to TPM units and normalization are described herein including with reference to FIG. 3.

Next, process 200 proceeds to act 206, where a blood cancer (BC) cytokine signature is generated for the subject using the RNA expression data obtained at act 202 (e.g., from bulk-sequencing data, converted to TPM units and subsequently log-normalized, as described herein including with reference to FIG. 3), and the RNA expression data obtained at act 204 (e.g., from bulk-sequencing data, converted to TPM units and subsequently log-normalized, as described herein including with reference to FIG. 3).

As described herein, in some embodiments, a BC cytokine signature comprises two or more (e.g., 2, 3, 4, 5, 6, 7, 8, 9, 10, 11, 12, 13, 14, 15, 16, 17, 18, 19, 20, 21, etc.) gene group scores. In some embodiments, the two or more gene group scores comprise gene group scores (which may also be referred to as gene group enrichment scores or gene group expression scores) for some or all of the gene groups shown in Table 2.

Accordingly, act 206 comprises: act 208 where the initial BC gene group scores are determined, and act 210 where the initial BC gene group scores determined at act 208 are normalized using RNA expression data for the cohort of subjects.

In some embodiments, determining the initial BC gene group scores comprises determining, for each of multiple (e.g., some or all of the) gene groups listed in Table 2, a respective gene group score. In some embodiments, determining the gene group scores comprises determining respective gene group scores for 2, 3, 4, 5, 6, 7, 8, 9, 10, 11, 12, 13, 14, 15, 16, 17, 18, 19, 20, or 21 gene groups (e.g., gene groups listed in Table 2). The gene group score for a particular gene group may be determined using RNA expression levels for at least some of the genes in the gene group (e.g., the RNA expression levels obtained at act 202). The RNA expression levels may be processed using a gene set enrichment analysis (GSEA) technique to determine the score for the particular gene group.

In some embodiments, determining the initial BC gene group scores comprises determining gene group scores for one or more gene groups associated with pro-tumor effects. In some embodiments, the one or more gene groups associated with pro-tumor effects are selected from among the tumor promotion gene groups, immune suppression gene groups, and/or stroma activation gene groups, listed in Table 2. In some embodiments, determining the BC gene group scores comprises determining gene group scores from one, some, or all of the tumor promotion gene groups (e.g., 0, 1, 2, 3, or 4 gene groups of the tumor promotion gene groups in Table 2), immune suppression gene groups (e.g., 0, 1, 2, 3, 4, or 5 gene groups of the immune suppression gene groups in Table 2), and/or stroma activation gene groups (e.g., 0, 1, or 2 gene groups of the stroma activation gene groups in Table 2), listed in Table 2.

In some embodiments, determining the initial BC gene group scores comprises determining BC gene group scores for one or more gene groups associated with anti-tumor effects. In some embodiments, the one or more gene groups associated with anti-tumor effects are selected from Type 1 response groups and tumor suppression groups listed in Table 2. In some embodiments, determining the BC gene group scores comprises determining gene group scores from one, some, or all of the Type 1 response gene groups (e.g., 0, 1, 2, 3, or 4 gene groups of the Type 1 response groups in Table 2) and/or tumor suppression gene groups (e.g., 0, 1, or 2 gene groups of the tumor suppression groups in Table 2) listed in Table 2.

In some embodiments, determining the initial BC gene group scores comprises determining BC gene group scores for one or more gene groups associated with B cell effects. In some embodiments, the one or more gene groups associated with B cell effects are selected from the pro-inflammatory cytokines group, pro-inflammatory cytokines FL group, myeloid cell recruitment group, and/or myeloid cell recruitment FL group in Table 2.

For example, in some embodiments, determining the cytokine signature comprises: determining initial gene group scores using the RNA expression levels for at least three genes from each of at least two of the gene groups, the gene groups including: Type 1 response groups (e.g., T cell recruitment group, cancer inhibiting inflammation group, Th1/M1 polarization and response group, Th1/M1 polarization and response FL group), tumor suppression groups (e.g., invasion and angiogenesis inhibition group, invasion and angiogenesis inhibition FL group), tumor promotion groups (e.g., lymphoma cell pro-survival group, cancer promoting inflammation group, cancer promoting inflammation FL group, promotion of lymphoma and dissemination group), immune suppression groups (e.g., Treg recruitment and function group, immunosuppressive factors group, immunosuppressive factors FL group, M2 polarization and response group, M2 polarization and response FL group), stroma activation groups (e.g., stroma and angiogenesis activation group, stroma and angiogenesis activation FL group), pro-inflammatory cytokines group, pro-inflammatory cytokines FL group, myeloid cell recruitment group, and/or myeloid cell recruitment FL group.

In some embodiments, determining the initial BC gene group scores comprises: determining gene group scores using the RNA expression levels for all genes in each of the following gene groups: Type 1 response groups (e.g., T cell recruitment group, cancer inhibiting inflammation group, Th1/M1 polarization and response group, Th1/M1 polarization and response FL group), tumor suppression groups (e.g., invasion and angiogenesis inhibition group, invasion and angiogenesis inhibition FL group), tumor promotion groups (e.g., lymphoma cell pro-survival group, cancer promoting inflammation group, cancer promoting inflammation FL group, promotion of lymphoma and dissemination group), immune suppression groups (e.g., Treg recruitment and function group, immunosuppressive factors group, immunosuppressive factors FL group, M2 polarization and response group, M2 polarization and response FL group), stroma activation groups (e.g., stroma and angiogenesis activation group, stroma and angiogenesis activation FL group), pro-inflammatory cytokines group, pro-inflammatory cytokines FL group, myeloid cell recruitment group, and/or myeloid cell recruitment FL group.

Aspects of determining the gene group scores are described herein, including with reference to FIG. 4 and in the Section titled “Cytokine Signatures”.

As described above, at act 210, the normalized BC gene group score is determined. In some embodiments, the normalized gene BC gene group score may be determined by normalizing the initial BC gene group scores relative to corresponding gene group scores generated using RNA expression data from a reference cohort of patients with the same BC type as the subject. This may be done in any suitable way. For example, the reference cohort may have N patients and normalizing a score for a particular gene group for a patient P (not in the reference cohort) may involve: (1) determining a gene group score for the same particular gene group for each of the N patients in the reference cohort to obtain a set of gene group scores for that reference cohort; (2) identifying the smallest and largest gene group score in the set of gene group scores for that reference cohort; (3) considering the smallest gene group score as 0% and the largest gene group score as 100% and dividing the range therebetween uniformly into percentages (e.g., if the smallest score is 120 and the largest score is 320, then 120-121 would correspond to 0%, 122-123 would correspond to 1%, 124-125 would correspond to 2%, and so on); and (4) determining the percentage in the range of scores for the reference cohort that corresponds to the score for the particular gene group (e.g., a score of 124.2 for patient P would map to 2% indicating that relative to the reference cohort, patient P's score is in the bottom 2%). It should be appreciated that the normalization may be done in other ways: using quantiles of the set of gene group scores for the reference cohort rather than a uniform division of the range, by computing a discrete cumulative distribution function (CDF) from the reference cohort scores and using an inverse of the CDF to identify a number between 0 and 1 for the score of the particular gene group, and/or in any other suitable way. In this way, a gene group score for a particular gene group can be turned into a normalized gene group score that represents a percentage relative to the reference cohort, which provides information about how large the magnitude of the gene group score for a particular patient is relative to the range of the same gene group scores seen for a reference cohort.

In some embodiments, the RNA expression data for the cohort may obtained from the TCGA project. However, the skilled artisan will recognize that other suitable sequencing datasets and/or RNA expression datasets from sources other than TCGA may be used to obtain RNA expression data for the cohort (so long as the cohort used has the same type of BC as the subject). In some embodiments, the normalized gene group scores of the subject may be assigned a “High”, “Medium”, or “Low” gene group score designation, according to thresholds set using the gene group scores of the cohort used for normalization. For example, if the normalized gene group score of a subject is less than a first threshold, the normalized gene group score may be identified as being “Low”. If the normalized gene group score of a subject is more than a first threshold and less than a second threshold, the normalized gene group score may be identified as being “Medium” (or “Med”). If the normalized gene group score of a subject normalized gene group score of a subject is more than the second threshold, the normalized gene group score may be identified as being “High”. In some embodiments, a normalized BC gene group score less than 17% is identified as being “Low”, more than 83% is identified as being “High”, and between 17%-83% is identified as being “Medium”.

Optionally, process 200 proceeds to act 212, where a visualization of the subject's cytokine signature may be generated. In some embodiments, generating the visualization of the cytokine signature comprises generating a graphical user interface (GUI) having a plurality of GUI elements, each of the GUI elements representing a respective normalized STC gene group score part of the cytokine signature. In some embodiments, a particular GUI element of the plurality GUI elements represents the respective normalized STC gene group score via a visual characteristic of the GUI element. In some embodiments, the visual characteristic is selected from the group consisting of a color, size, and font. In some embodiments, the GUI may be interactive in that a user can indicate a selection of one or more of the GUI elements (e.g., a GUI element representing a particular gene group) and receive more information in response to that selection (e.g., information indicating the normalized gene scores for one or more (e.g., all) genes in the particular gene group).

For example, in some embodiments, the visualization of the cytokine signature may be a GUI showing a “solar” diagram. The diagram may be used to illustrate the intensity of different cytokine-mediated biological processes represented by the different gene groups. The solar diagram provides a visual representation of the normalized gene group scores for the gene groups. In some embodiments, the solar diagram represents the normalized gene group scores by respective ray so that each ray corresponds to a respective gene group and a visual characteristic of the ray depends on the normalized gene group score for that gene group. Each normalized gene group score may take on a value representing a percentage between 0 and 100% (or, e.g., a number between 0 and 1 or any other suitable scale to represent a percentage). For example, as shown in FIG. 6, the height of the rays represents the normalized gene group scores of the gene groups represented by those rays. For instance, the heights of rays 602, 604, and 606 represent the normalized STC gene group scores of the Eosinophil/Basophil activation gene group, the Eosinophil/Basophil recruitment gene group, and the Tumor growth arrest group, respectively. As can be seen in this visualization, the normalized gene group score of the Eosinophil/Basophil activation gene group (represented by ray 602) is greater than the normalized gene group score of the Eosinophil/Basophil recruitment gene group (represented by ray 604).

As can also be seen in FIG. 6, the solar diagram includes concentric rings indicative of a hierarchical grouping of the gene groups. The inner-most ring, for example, groups all the outer-most gene groups into three “high-level” sets of groups: “Anti-tumor effects”, “Pro-tumor effects” and “Others”. The second ring, for example, groups all the outer-most gene groups into 9 “mid-level” sets of groups: “Tumor suppression”, “Type 1 response”, “Fibrosis, angiogenesis”, “Tumor progression”, “Immune suppression”, “Type 2 response”, “Myeloid inflammation”, “Immune cell recruitment”, and “B cell response”. The constituent gene groups of these mid-level sets of groups are shown in the next ring. The skilled artisan will recognize that the same “high-level”, “mid-level”, and outer ring gene groups shown for STC cytokine signatures in FIG. 6 may be applied to the BC gene groups set forth in Table 2 and visualized in the same way.

As can also be seen in FIG. 6, the color, shading, or other characteristics can be used to indicate whether a gene group or a set of gene groups is pro-tumor or anti-tumor.

In some embodiments, process 200 proceeds to act 214, where the subject's likelihood of responding to a therapy is identified using the cytokine signature identified at act 206. Aspects of identifying whether or not a subject is likely to respond to a therapy are described herein including in the section below titled “Therapeutic Indications.”

In some embodiments, process 200 completes after act 214 completes. In some such embodiments, the determined cytokine signature (or visualization of cytokine signature generated at act 212), and/or the identified likelihood the subject will respond to a therapy may be stored for subsequent use, provided to one or more recipients (e.g., a clinician, a researcher, etc.).

However, in some embodiments, one or more other acts are performed after act 214. For example, in the illustrated embodiment of FIG. 2, process 200 may include one or more of optional acts 212, 214, and 216 shown using dashed lines in FIG. 2. For example, when a subject is identified as having an increased likelihood of responding to immunotherapy at act 214, the subject is administered one or more immunotherapies at act 216. Examples of immunotherapies and other therapies are provided herein.

It should be appreciated that although acts 212, 214, and 216 are indicated as optional in the example of FIG. 2, in other embodiments, one or more other acts may be optional (in addition to or instead of acts 212, 214, and 216). For example, in some embodiments, acts 202 and 204 may be optional (e.g., when the RNA expression data is obtained previously, process 200 may begin at act 206 by accessing the previously obtained RNA expression data). In some embodiments, the process 200 may comprise acts 202, 204, 206, 208, 210, 212 and 214, without act 216. In some embodiments, the process 200 may comprise acts 202, 204, 206, 208, 210, 214, and 216, without act 212.

TABLE 2
List of gene groups for blood cancer (BC) cytokine signatures

Genes

Anti-tumor effects groups
Type 1 response groups

| | |
|---|---|
| T cell recruitment group | IL15, CXCL11, CCL5, CX3CL1, CCL3, IFNG, CXCL10, |
| | CCL19, CCL21, CXCL9, CCL4 |
| Cancer inhibiting inflammation | IL1B, IFNG, CCL3, CCL4, CCL5, CXCL10, FLT3LG, XCL1, |
| group | XCL2, IL15, CCL19, CCL21, CXCL9, IL18 |
| Th1/M1 polarization and | TNF, CSF2, IL12A, IL12B, IL23A, IL6, IL1B, IFNG, |
| response group | CXCL10, CXCL9, IL27, CCL2, CCL13, CCL18, IL15 |
| Th1/M1 polarization and | TNF, CSF2, IL23A, IL1B, IFNG, CXCL10, CXCL9, IL27, |
| response FL group | CCL2, IL15 |

Tumor suppression groups

| | |
|---|---|
| Invasion and angiogenesis | THBS1, THBS2, THBS3, THBS4 COL18A1 SERPINF1 |
| inhibition group | VASH1, MMRN2, TIMP2, TIMP3 |
| Invasion and angiogenesis | THBS1, THBS2, THBS3, THBS4 COL18A1 SERPINF1 |
| inhibition FL group | VASH1 MMRN2, TIMP2, TIMP3, TIMP1 |

Pro-tumor effects groups
Tumor promotion groups

| | |
|---|---|
| Lymphoma cell pro-survival | TNFSF13B, TNFSF13, IL15, IL10, VEGFA, IL32, CD40LG, |
| signal group | IL21 IL33 |
| Cancer promoting inflammation | IL6, IL21, EBI3, TGFB1, CXCL12, CSF1, CSF2, VEGFA, |
| group | IDO1, S100A8, S100A9, CCL2, CCL5, CXCL8 |
| Cancer promoting inflammation | IL10, IL21, EBI3, TGFB1, CSF1, VEGFA , IDO1, CCL2, |
| FL group | CCL5 |
| Promotion of lymphoma | CXCL13, FGF2, TGFB1, MMP9, CXCL10, HGF S100A11 |
| dissemination group | IFI30 HK3 |

Immune suppression groups

| | |
|---|---|
| Treg recruitment and function | CCL17, CCL22, LGALS9, CD40LG, CXCL13 |
| group | |
| Immunosuppressive factors | LGALS1, TGFB3, IDO1, PTGES, TGFB1, CCL2, CXCL2, |
| group | CXCL5, CCL22, EBI3, LGALS9, CSF1 |
| Immunosuppressive factors FL | LGALS1, TGFB3, IDO1, IL10, TGFB1, EBI3, CSF1 |
| group | |
| M2 polarization and response | IL21 TGFB1 IL33, CSF2 LIF, TGFB3 TNFSF13B, |
| group | TNFSF13, TGFB3 |
| M2 polarization and response | IL10, IL21, IL33, CSF2, LIF, TGFB3, IL4, TNFSF13B, |
| FL group | TNFSF13 |

Stroma activation groups

| | |
|---|---|
| Stroma and angiogenesis | IL6, IL1B, VEGFA, PIGF, PGF, VEGFC, FGF2, HGF, |
| activation group | PDGFA, PDGFB, SPP1, ANGPT1, LTA |
| Stroma and angiogenesis | IL1B, VEGFA, PGF, VEGFC, FGF2, HGF, PDGFA, PDGFB, |
| activation FL group | SPP1, ANGPT1, LTA |

B cell effects groups

| | |
|---|---|
| Pro-inflammatory cytokines | IL15, CCL5, CCL2, CCL3, CXCL8, TNF, IL6, IFNG, IL1B |
| group | CXCL1, CXCL2, CXCL9, CXCL10 |
| Pro-inflammatory cytokines FL | IL15, CCL5, CCL2, CCL3, CXCL8, TNF, IFNG, IL1B, |
| group | CXCL1, CXCL2, CXCL9, CXCL10 |
| Myeloid cell recruitment group | CXCL8, CCL2, CSF1, CCL8, CXCL12, HBEGF, S100A9, |
| | S100A8 |
| Myeloid cell recruitment FL | CXCL8, CCL2, CSF1, CCL8, HBEGF, S100A9 |
| group | |

### Biological Samples

Aspects of the disclosure relate to methods for identifying the cytokine signature of a subject by analyzing gene expression data obtained from a biological sample that has been obtained from the subject.

The biological sample may be from any source in the subject's body including, but not limited to, any fluid [such as blood (e.g., whole blood, blood serum, or blood plasma), saliva, tears, synovial fluid, cerebrospinal fluid, pleural fluid, pericardial fluid, ascitic fluid, and/or urine], hair, skin (including portions of the epidermis, dermis, and/or hypodermis), oropharynx, laryngopharynx, esophagus, stomach, bronchus, salivary gland, tongue, oral cavity, nasal cavity, vaginal cavity, anal cavity, bone, bone marrow, brain, thymus, spleen, small intestine, appendix, colon, rectum, anus, liver, biliary tract, pancreas, kidney, ureter, bladder, urethra, uterus, vagina, vulva, ovary, cervix, scrotum, penis, prostate, testicle, seminal vesicles, and/or any type of tissue (e.g., muscle tissue, epithelial tissue, connective tissue, or nervous tissue).

The biological sample may be any type of sample including, for example, a sample of a bodily fluid, one or more cells, a piece of tissue, or some or all of an organ. In some embodiments, a tissue sample may be obtained from a subject using a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy).

A sample of lymph node or blood, in some embodiments, refers to a sample comprising cells, e.g., cells from a blood sample or lymph node sample. In some embodiments, the sample comprises non-cancerous cells. In some embodiments, the sample comprises pre-cancerous cells. In some embodiments, the sample comprises cancerous cells. In some embodiments, the sample comprises blood cells. In some embodiments, the sample comprises lymph node cells. In some embodiments, the sample comprises lymph node cells and blood cells.

A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot.

In some embodiments, a sample of blood is collected to obtain the cell-free nucleic acid (e.g., cell-free DNA) in the blood.

In some embodiments, the sample may be from a cancerous tissue or organ or a tissue or organ suspected of having one or more cancerous cells. In some embodiments, the sample may be from a healthy (e.g., non-cancerous) tissue or organ. In some embodiments, a sample from a subject (e.g., a biopsy from a subject) may include both healthy and cancerous cells and/or tissue. In certain embodiments, one sample will be taken from a subject for analysis. In some embodiments, more than one (e.g., 2, 3, 4, 5, 6, 7, 8, 9, 10, 11, 12, 13, 14, 15, 16, 17, 18, 19, 20, or more) samples may be taken from a subject for analysis. In some embodiments, one sample from a subject will be analyzed. In certain embodiments, more than one (e.g., 2, 3, 4, 5, 6, 7, 8, 9, 10, 11, 12, 13, 14, 15, 16, 17, 18, 19, 20, or more) samples may be analyzed. If more than one sample from a subject is analyzed, the samples may be procured at the same time (e.g., more than one sample may be taken in the same procedure), or the samples may be taken at different times (e.g., during a different procedure including a procedure 1, 2, 3, 4, 5, 6, 7, 8, 9, 10 days; 1, 2, 3, 4, 5, 6, 7, 8, 9, 10 weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure). A second or subsequent sample may be taken or obtained from the same region (e.g., from the same tumor or area of tissue) or a different region (including, e.g., a different tumor). A second or subsequent sample may be taken or obtained from the subject after one or more treatments, and may be taken from the same region or a different region. As a non-limiting example, the second or subsequent sample may be useful in determining whether the cancer in each sample has different characteristics (e.g., in the case of samples taken from two physically separate tumors in a patient) or whether the cancer has responded to one or more treatments (e.g., in the case of two or more samples from the same tumor prior to and subsequent to a treatment).

Any of the biological samples described herein may be obtained from the subject using any known technique. In some embodiments, the biological sample may be obtained from a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy). In some embodiments, each of the at least one biological sample is a bodily fluid sample, a cell sample, or a tissue biopsy.

Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one form to another form such that the first form is no longer detected at the same level as before degradation.

In some embodiments, the biological sample is stored using cryopreservation. Non-limiting examples of cryopreservation include, but are not limited to, step-down freezing, blast freezing, direct plunge freezing, snap freezing, slow freezing using a programmable freezer, and vitrification. In some embodiments, the biological sample is stored using lyophilization. In some embodiments, a biological sample is placed into a container that already contains a preservant (e.g., RNALater to preserve RNA) and then frozen (e.g., by snap-freezing), after the collection of the biological sample from the subject. In some embodiments, such storage in frozen state is done immediately after collection of the biological sample. In some embodiments, a biological sample may be kept at either room temperature or 4° C. for some time (e.g., up to an hour, up to 8 h, or up to 1 day, or a few days) in a preservant or in a buffer without a preservant, before being frozen.

Non-limiting examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM Tris-Cl; 0.5 mM EDTA, pH 9.0)) and other coagulants, and Acids Citrate Dextrose (e.g., for blood specimens).

In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Any of the biological samples from a subject described herein may be stored under any condition that preserves stability of the biological sample. In some embodiments, the biological sample is stored at a temperature that preserves stability of the biological sample. In some embodiments, the sample is stored at room temperature (e.g., 25° C.). In some embodiments, the sample is stored under refrigeration (e.g., 4° C.). In some embodiments, the sample is stored under freezing conditions (e.g., −20° C.). In some embodiments, the sample is stored under ultralow temperature conditions (e.g., −50° C. to −800° C.). In some embodiments, the sample is stored under liquid nitrogen (e.g., −170° C.). In some embodiments, a biological sample is stored at −60° C. to −8° C. (e.g., −70° C.) for up to 5 years (e.g., up to 1 month, up to 2 months, up to 3 months, up to 4 months, up to 5 months, up to 6 months, up to 7 months, up to 8 months, up to 9 months, up to 10 months, up to 11 months, up to 1 year, up to 2 years, up to 3 years, up to 4 years, or up to 5 years). In some embodiments, a biological sample is stored as described by any of the methods described herein for up to 20 years (e.g., up to 5 years, up to 10 years, up to 15 years, or up to 20 years).

### Obtaining RNA Expression Data

Aspects of the disclosure relate to methods of determining a cytokine signature of a subject using sequencing data or RNA expression data obtained from a biological sample from the subject.

The RNA expression data used in methods described herein typically is derived from sequencing data obtained from the biological sample.

The sequencing data may be obtained from the biological sample using any suitable sequencing technique and/or apparatus. In some embodiments, the sequencing apparatus used to sequence the biological sample may be selected from any suitable sequencing apparatus known in the art including, but not limited to, Illumina™, SOLid™, Ion Torrent™, PacBio™, a nanopore-based sequencing apparatus, a Sanger sequencing apparatus, or a 454™ sequencing apparatus. In some embodiments, sequencing apparatus used to sequence the biological sample is an Illumina sequencing (e.g., NovaSeq™, NextSeq™, HiSeq™, MiSeq™, or MiniSeq™) apparatus.

After the sequencing data is obtained, it is processed in order to obtain the RNA expression data. RNA expression data may be acquired using any method known in the art including, but not limited to whole transcriptome sequencing, whole exome sequencing, total RNA sequencing, mRNA sequencing, targeted RNA sequencing, RNA exome capture sequencing, next generation sequencing, and/or deep RNA sequencing. In some embodiments, RNA expression data may be obtained using a microarray assay.

In some embodiments, the sequencing data is processed to produce RNA expression data. In some embodiments, RNA sequence data is processed by one or more bioinformatics methods or software tools, for example RNA sequence quantification tools (e.g., Kallisto) and genome annotation tools (e.g., Gencode v23), in order to produce expression data. The Kallisto software is described in Nicolas L Bray, Harold Pimentel, Pill Melsted and Lior Pachter, Near-optimal probabilistic RNA-seq quantification, Nature Biotechnology 34, 525-527 (2016), doi:10.1038/nbt.3519, which is incorporated by reference in its entirety herein.

In some embodiments, microarray expression data is processed using a bioinformatics R package, such as “affy” or “limma,” in order to produce expression data. The “affy” software is described in Bioinformatics. 2004 Feb. 12; 20(3):307-15. doi: 10.1093/bioinformatics/btg405. “affy—analysis of Affymetrix GeneChip data at the probe level” by Laurent Gautier 1, Leslie Cope, Benjamin M Bolstad, Rafael A Irizarry PMID: 14960456 DOI: 10.1093/bioinformatics/btg405, which is incorporated by reference herein in its entirety. The “limma” software is described in Ritchie M E, Phipson B, Wu D, Hu Y, Law C W, Shi W, Smyth G K “limma powers differential expression analyses for RNA-sequencing and microarray studies.” Nucleic Acids Res. 2015 Apr. 20; 43(7):e47. 20. doi.org/10.1093/nar/gkv007PMID: 25605792, PMCID: PMC4402510, which is incorporated by reference herein its entirety.

In some embodiments, sequencing data and/or expression data comprises more than 5 kilobases (kb). In some embodiments, the size of the obtained RNA data is at least 10 kb. In some embodiments, the size of the obtained RNA sequencing data is at least 100 kb. In some embodiments, the size of the obtained RNA sequencing data is at least 500 kb. In some embodiments, the size of the obtained RNA sequencing data is at least 1 megabase (Mb). In some embodiments, the size of the obtained RNA sequencing data is at least 10 Mb. In some embodiments, the size of the obtained RNA sequencing data is at least 100 Mb. In some embodiments, the size of the obtained RNA sequencing data is at least 500 Mb. In some embodiments, the size of the obtained RNA sequencing data is at least 1 gigabase (Gb). In some embodiments, the size of the obtained RNA sequencing data is at least 10 Gb. In some embodiments, the size of the obtained RNA sequencing data is at least 100 Gb. In some embodiments, the size of the obtained RNA sequencing data is at least 500 Gb.

In some embodiments, the expression data is acquired through bulk RNA sequencing. Bulk RNA sequencing may include obtaining expression levels for each gene across RNA extracted from a large population of input cells (e.g., a mixture of different cell types.) In some embodiments, the expression data is acquired through single cell sequencing (e.g., scRNA-seq). Single cell sequencing may include sequencing individual cells.

In some embodiments, bulk sequencing data comprises at least 1 million reads, at least 5 million reads, at least 10 million reads, at least 20 million reads, at least 50 million reads, or at least 100 million reads. In some embodiments, bulk sequencing data comprises between 1 million reads and 5 million reads, 3 million reads and 10 million reads, 5 million reads and 20 million reads, 10 million reads and 50 million reads, 30 million reads and 100 million reads, or 1 million reads and 100 million reads (or any number of reads including, and between).

In some embodiments, the expression data comprises next-generation sequencing (NGS) data. In some embodiments, the expression data comprises microarray data.

Expression data (e.g., indicating expression levels) for a plurality of genes may be used for any of the methods or compositions described herein. The number of genes which may be examined may be up to and inclusive of all the genes of the subject. In some embodiments, expression levels may be determined for all of the genes of a subject. As a non-limiting example, four or more, five or more, six or more, seven or more, eight or more, nine or more, ten or more, eleven or more, twelve or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 35 or more, 40 or more, 50 or more, 60 or more, 70 or more, 80 or more, 90 or more, 100 or more, 125 or more, 150 or more, 175 or more, 200 or more, 225 or more, 250 or more, 275 or more, or 300 or more genes may be used for any evaluation described herein. As another set of non-limiting examples, the expression data may include, for each gene group listed in Table 1 or Table 2, expression data for at least 5, at least 10, at least 15, or at least 20 genes selected from each gene group.

In some embodiments, RNA expression data is obtained by accessing the RNA expression data from at least one computer storage medium on which the RNA expression data is stored. Additionally or alternatively, in some embodiments, RNA expression data may be received from one or more sources via a communication network of any suitable type. For example, in some embodiment, the RNA expression data may be received from a server (e.g., a SFTP server, or Illumina BaseSpace).

The RNA expression data obtained may be in any suitable format, as aspects of the technology described herein are not limited in this respect. For example, in some embodiments, the RNA expression data may be obtained in a text-based file (e.g., in a FASTQ, FASTA, BAM, or SAM format). In some embodiments, a file in which sequencing data is stored may contains quality scores of the sequencing data. In some embodiments, a file in which sequencing data is stored may contain sequence identifier information.

Expression data, in some embodiments, includes gene expression levels. Gene expression levels may be detected by detecting a product of gene expression such as mRNA and/or protein. In some embodiments, gene expression levels are determined by detecting a level of a mRNA in a sample. As used herein, the terms “determining” or “detecting” may include assessing the presence, absence, quantity and/or amount (which can be an effective amount) of a substance within a sample, including the derivation of qualitative or quantitative concentration levels of such substances, or otherwise evaluating the values and/or categorization of such substances in a sample from a subject.

FIG. 3 shows an exemplary process 102 for obtaining sequencing data and processing the sequencing data to obtain RNA expression data from sequencing data. Process 102 may be performed by any suitable computing device or devices, as aspects of the technology described herein are not limited in this respect. For example, process 102 may be performed by a computing device part of a sequencing apparatus. In other embodiments, process 102 may be performed by one or more computing devices external to the sequencing apparatus.

Process 102 begins at act 300, where sequencing data is obtained from a biological sample obtained from a subject and processed to obtain RNA expression data. The sequencing data is obtained by any suitable method, for example, using any of the methods described herein including in the Section titled “Biological Samples.”

In some embodiments, the processed sequencing data (e.g., RNA expression data) obtained at act 300 comprises RNA-seq data. In some embodiments, the biological sample comprises blood or tissue. In some embodiments, the biological sample comprises one or more tumor cells, for example, one or more bladder tumor cells.

Next, process 102 proceeds to act 302 where the RNA expression data obtained at act 300 is normalized to transcripts per kilobase million (TPM) units. The normalization may be performed using any suitable software and in any suitable way. For example, in some embodiments, TPM normalization may be performed according to the techniques described in Wagner et al. (Theory Biosci. (2012) 131:281-285), which is incorporated by reference herein in its entirety. In some embodiments, the TPM normalization may be performed using a software package, such as, for example, the gcrma package. Aspects of the gcrma package are described in Wu J, Gentry RIwcfJMJ (2021). “gcrma: Background Adjustment Using Sequence Information. R package version 2.66.0.,” which is incorporated by reference in its entirety herein. In some embodiments, RNA expression level in TPM units for a particular gene may be calculated according to the following formula:

$$A \cdot \frac{1}{\sum ( A )} \cdot {10}^{6} \\ Where A = \frac{total reads mapped to gene \cdot {10}^{3}}{gene length in bp}$$

Next, process 102 proceeds to act 304, where the RNA expression levels in TPM units (as determined at act 302) may be log transformed. Process 102 is illustrative and there are variations. For example, in some embodiments, one or both of acts 302 and 304 may be omitted. Thus, in some embodiments, the RNA expression levels may not be normalized to transcripts per million units and may, instead, be converted to another type of unit (e.g., reads per kilobase million (RPKM) or fragments per kilobase million (FPKM) or any other suitable unit). Additionally or alternatively, in some embodiments, the log transformation may be omitted. Instead, no transformation may be applied in some embodiments, or one or more other transformations may be applied in lieu of the log transformation.

RNA expression data obtained by process 102 can include the sequence data generated by a sequencing protocol (e.g., the series of nucleotides in a nucleic acid molecule identified by next-generation sequencing, sanger sequencing, etc.) as well as information contained therein (e.g., information indicative of source, tissue type, etc.) which may also be considered information that can be inferred or determined from the sequence data. In some embodiments, expression data obtained by process 102 can include information included in a FASTA file, a description and/or quality scores included in a FASTQ file, an aligned position included in a BAM file, and/or any other suitable information obtained from any suitable file.

The skilled person recognizes that although the process of obtaining sequencing data, processing the sequencing data to obtain RNA expression data, and normalization of the RNA expression data to TPM units has been described with respect to process 102, processes 104, 202, and 204 in FIG. 1 and FIG. 2 may comprise the same (or substantially the same) acts.

### Cytokine Signatures

Aspects of the disclosure relate to processing of expression data to determine one or more gene expression signatures (e.g., gene group scores comprising a cytokine signature). In some embodiments, expression data (e.g., RNA expression data) is processed using a computing device to determine the one or more gene expression signatures. In some embodiments, the computing device may be operated by a user such as a doctor, clinician, researcher, patient, or other individual. For example, the user may provide the expression data as input to the computing device (e.g., by uploading a file), and/or may provide user input specifying processing or other methods to be performed using the expression data.

In some embodiments, expression data may be processed by one or more software programs running on computing device.

In some embodiments, methods described herein comprise an act of determining initial STC or BC gene group scores for respective gene groups in a plurality of gene groups. In some embodiments, a cytokine signature comprises gene group scores for at least one (e.g., 1, 2, 3, 4, 5, 6, 7, 8, 9, 10, 11, 12, 13, 14, 15, 16, 17, 18, 19, 20, 21, 22, 23, 24, 25, 26, 27, or 28) of the gene groups listed in Table 1 or Table 2.

The number of genes in a gene group used to determine a gene group score may vary. In some embodiments, all RNA expression levels for all genes in a particular gene group may be used to determine a gene group score for the particular gene group. In other embodiments, RNA expression data for fewer than all genes may be used (e.g., RNA expression levels for at least two genes, at least three genes, at least five genes, between 2 and 10 genes, between 5 and 15 genes, between 3 and 20 genes, or any other suitable range within these ranges).

In some embodiments, initial STC gene group scores comprise a gene group score for the CTL and Th1 cells activation group. In some embodiments, this initial gene group score may be calculated using RNA expression levels of at least three genes (e.g., at least 3, 4, 5, 6, 7, 8, 9, 10, or 11) in the CTL and Th1 cells activation group, which is defined by its constituent genes: IFNG, IL18, IL15RA, TNF, IL27, CCL5, TNFSF11, CD40LG, FLT3LG, TNFSF9, and CD70.

In some embodiments, initial STC gene group scores comprise a gene group score for the M1 polarization group. In some embodiments, this initial gene group score may be calculated using RNA expression levels of at least three genes (e.g., at least 3, 4, 5, 6, or 7) in the M1 polarization group, which is defined by its constituent genes: CCL21, IFNG, CSF2, TNF, IL23A, IL12B, and IL27.

In some embodiments, initial STC gene group scores comprise a gene group score for the TLS formation group. In some embodiments, this initial gene group score may be calculated using RNA expression levels of at least three genes (e.g., at least 3, 4, 5, 6, 7, 8, 9, 10, 11, or 12) in the TLS formation group, which is defined by its constituent genes: CXCL11, CCL18, CXCL10, CXCL9, CCL2, CXCL13, CCL8, CCL5, CCL4, CCL3, CCL19, and CCL21.

In some embodiments, initial STC gene group scores comprise a gene group score for the tumor growth and arrest group. In some embodiments, this initial gene group score may be calculated using RNA expression levels of at least three genes (e.g., at least 3, 4, 5, 6, 7, or 8) in the tumor growth and arrest group, which is defined by its constituent genes: IFNG, CXCR2, ACKR2, ACKR4, ACKR1, BAMBI, TNFSF10, and LTA.

In some embodiments, initial STC gene group scores comprise a gene group score for the metastasis inhibition group. In some embodiments, this initial gene group score may be calculated using RNA expression levels of at least three genes (e.g., at least 3, or 4) in the metastasis inhibition group, which is defined by its constituent genes: ACKR1, ACKR2, ACKR4, and CCL28.

In some embodiments, initial STC gene group scores comprise a gene group score for the angiogenesis inhibition group. In some embodiments, this initial gene group score may be calculated using RNA expression levels of at least three genes (e.g., at least 3, 4, 5, 6, 7, 8, 9, 10, 11, or 12) in the angiogenesis inhibition group, which is defined by its constituent genes: IFNG, IL12A, IL12B, CXCL9, CXCL10, CXCL11, CXCR3, CCL5, CCR5, ACKR1, ARRB2, and TNFSF15.

In some embodiments, initial STC gene group scores comprise a gene group score for the B cell activation group. In some embodiments, this initial gene group score may be calculated using RNA expression levels of at least three genes (e.g., at least 3, 4, or 5) in the B cell activation group, which is defined by its constituent genes: IFNG, IL4, IL10, TNFSF13B, and TNFSF13.

In some embodiments, initial STC gene group scores comprise a gene group score for the lymphocyte recruitment group. In some embodiments, this initial gene group score may be calculated using RNA expression levels of at least three genes (e.g., at least 3, 4, 5, 6, 7, 8, 9, or 10) in the lymphocyte recruitment group, which is defined by its constituent genes: CXCL9, CXCL10, CXCL11, CCL5, CXCL13, CCL20, CCL21, CCL19, CCL17, and CCL22.

In some embodiments, initial STC gene group scores comprise a gene group score for the macrophage and DC recruitment group. In some embodiments, this initial gene group score may be calculated using RNA expression levels of at least three genes (e.g., at least 3, 4, 5, 6, or 7) in the macrophage and DC recruitment group, which is defined by its constituent genes: CCL2, CCL3, TGFB1, CSF1, XCL1, CXCL12, and CCL18.

In some embodiments, initial STC gene group scores comprise a gene group score for the pro-inflammatory cytokines group. In some embodiments, this initial gene group score may be calculated using RNA expression levels of at least three genes (e.g., at least 3, 4, 5, 6, 7, 8, 9, 10, or 11) in the pro-inflammatory cytokines group, which is defined by its constituent genes: IL1A, IL1B, TNF, IL6, IL23A, LIF, CCL2, CCL3, CCL4, CXCL2, and CXCL1.

In some embodiments, initial STC gene group scores comprise a gene group score for the neutrophil recruitment and activation group. In some embodiments, this initial gene group score may be calculated using RNA expression levels of at least three genes (e.g., at least 3, 4, 5, 6, 7, 8, 9, or 10) in the neutrophil recruitment and activation group, which is defined by its constituent genes: CXCL1, CXCL2, CXCL3, PF4, CXCL5, CXCL8, CSF2, CSF3, PPBP, and CXCL6.

In some embodiments, initial STC gene group scores comprise a gene group score for the Th2 response group. In some embodiments, this initial gene group score may be calculated using RNA expression levels of at least three genes (e.g., at least 3, 4, 5, 6, or 7) in the Th2 response group, which is defined by its constituent genes: IL4, IL10, IL11, IL13, CCL8, TSLP, and CCL13.

In some embodiments, initial STC gene group scores comprise a gene group score for the M2 polarization group. In some embodiments, this initial gene group score may be calculated using RNA expression levels of at least three genes (e.g., at least 3, 4, 5, or 6) in the M2 polarization group, which is defined by its constituent genes: IL10, IL33, CCL18, CCL24, TGFB2, and TGFB3.

In some embodiments, initial STC gene group scores comprise a gene group score for the eosinophil/basophil recruitment group. In some embodiments, this initial gene group score may be calculated using RNA expression levels of at least three genes (e.g., at least 3, 4, or 5) in the eosinophil/basophil recruitment group, which is defined by its constituent genes: CCL8, CCL11, CCL13, CCL24, and CCL26.

In some embodiments, initial STC gene group scores comprise a gene group score for the eosinophil/basophil activation group. In some embodiments, this initial gene group score may be calculated using RNA expression levels of at least three genes (e.g., at least 3, 4, 5, or 6) in the eosinophil/basophil activation group, which is defined by its constituent genes: IL33, ENPP3, IL1RL1, IL5RA, ANGPT1, and IL13.

In some embodiments, initial STC gene group scores comprise a gene group score for the stromal suppressive factors group. In some embodiments, this initial gene group score may be calculated using RNA expression levels of at least three genes (e.g., at least 3, 4, 5, 6, or 7) in the stromal suppressive factors group, which is defined by its constituent genes: TGFB2, TGFB3, TDO2, TGFBI, IL6, IL11, and TSLP.

In some embodiments, initial STC gene group scores comprise a gene group score for the myeloid suppressive factors group. In some embodiments, this initial gene group score may be calculated using RNA expression levels of at least three genes (e.g., at least 3, 4, 5, 6, or 7) in the myeloid suppressive factors group, which is defined by its constituent genes: IL10, FGL2, IDO1, EBI3, IL4I1, TGFB1, and TGFBI.

In some embodiments, initial STC gene group scores comprise a gene group score for the CTL exclusion group. In some embodiments, this initial gene group score may be calculated using RNA expression levels of at least three genes (e.g., at least 3, 4, 5, 6, 7, 8, or 9) in the CTL exclusion group, which is defined by its constituent genes: CXCL12, EDNRB, PDGFC, FGF2, GAS6, TNFAIP6, TGFBI, VEGFA, and AXL.

In some embodiments, initial STC gene group scores comprise a gene group score for the Treg polarization group. In some embodiments, this initial gene group score may be calculated using RNA expression levels of at least three genes (e.g., at least 3, 4, 5, 6, 7, or 8) in the Treg polarization group, which is defined by its constituent genes: TGFB1, TGFB2, TGFB3, IL2RA, IL10, IDO1, CCL17, and CCL22.

In some embodiments, initial STC gene group scores comprise a gene group score for the tumor growth promotion group. In some embodiments, this initial gene group score may be calculated using RNA expression levels of at least three genes (e.g., at least 3, 4, 5, 6, or 7) in the tumor growth promotion group, which is defined by its constituent genes: IL6, HGF, TGFB3, FGF7, FGF10, IGF1, and EGF.

In some embodiments, initial STC gene group scores comprise a gene group score for the induction of EMT group. In some embodiments, this initial gene group score may be calculated using RNA expression levels of at least three genes (e.g., at least 3, 4, 5, 6, or 7) in the induction of EMT group, which is defined by its constituent genes: IL6, LIF, CCL7, CCL8, CXCL8, TNFSF11, and ARTN.

In some embodiments, initial STC gene group scores comprise a gene group score for the metastasis promotion group. In some embodiments, this initial gene group score may be calculated using RNA expression levels of at least three genes (e.g., at least 3, 4, 5, 6, 7, 8, 9, 10, or 11) in the metastasis promotion group, which is defined by its constituent genes: OSM, IL1B, CCR4, CCR6, CXCR4, HGF, CXCL8, TNFSF11, TGFB1, IL15RA, and HIF1A.

In some embodiments, initial STC gene group scores comprise a gene group score for the angiogenesis induction group. In some embodiments, this initial gene group score may be calculated using RNA expression levels of at least three genes (e.g., at least 3, 4, 5, 6, 7, or 8) in the angiogenesis induction group, which is defined by its constituent genes: VEGFA, VEGFC, FGF2, PDGFA, PDGFB, PDGFC, TGFB1, and TNFSF12.

In some embodiments, initial STC gene group scores comprise a gene group score for the CAF recruitment group. In some embodiments, this initial gene group score may be calculated using RNA expression levels of at least three genes (e.g., at least 3, 4, 5, 6, 7, or 8) in the CAF recruitment group, which is defined by its constituent genes: FGF1, FGF2, TGFB1, TGFB2, TGFB3, CXCL12, IL6, and MMP2.

In some embodiments, initial BC gene group scores comprise a gene group score for the T cell recruitment group. In some embodiments, this initial gene group score may be calculated using RNA expression levels of at least three genes (e.g., at least 3, 4, 5, 6, 7, 8, 9, 10, or 11) in the T cell recruitment group, which is defined by its constituent genes: IL15, CXCL11, CCL5, CX3CL1, CCL3, IFNG, CXCL10, CCL19, CCL21, CXCL9, and CCL4.

In some embodiments, initial BC gene group scores comprise a gene group score for the cancer inhibiting inflammation group. In some embodiments, this initial gene group score may be calculated using RNA expression levels of at least three genes (e.g., at least 3, 4, 5, 6, 7, 8, 9, 10, 11, 12, 13, or 14) in the cancer inhibiting inflammation group, which is defined by its constituent genes: IL1B, IFNG, CCL3, CCL4, CCL5, CXCL10, FLT3LG, XCL1, XCL2, IL15, CCL19, CCL21, CXCL9, and IL18.

In some embodiments, initial BC gene group scores comprise a gene group score for the Th1/M1 polarization and response group. In some embodiments, this initial gene group score may be calculated using RNA expression levels of at least three genes (e.g., at least 3, 4, 5, 6, 7, 8, 9, 10, 11, 12, 13, 14, or 15) in the Th1/M1 polarization and response group, which is defined by its constituent genes: TNF, CSF2, IL12A, IL12B, IL23A, IL6, IL1B, IFNG, CXCL10, CXCL9, IL27, CCL2, CCL13, CCL18, and IL15.

In some embodiments, initial BC gene group scores comprise a gene group score for the Th1/M1 polarization and response FL group. In some embodiments, this initial gene group score may be calculated using RNA expression levels of at least three genes (e.g., at least 3, 4, 5, 6, 7, 8, 9, or 10) in the Th1/M1 polarization and response FL group, which is defined by its constituent genes: TNF, CSF2, IL23A, IL1B, IFNG, CXCL10, CXCL9, IL27, CCL2, and IL15.

In some embodiments, initial BC gene group scores comprise a gene group score for the invasion and angiogenesis inhibition group. In some embodiments, this initial gene group score may be calculated using RNA expression levels of at least three genes (e.g., at least 3, 4, 5, 6, 7, 8, 9, or 10) in the invasion and angiogenesis inhibition group, which is defined by its constituent genes: THBS1, THBS2, THBS3, THBS4, COL18A1, SERPINF1 VASH1, MMRN2, TIMP2, and TIMP3.

In some embodiments, initial BC gene group scores comprise a gene group score for the invasion and angiogenesis inhibition FL group. In some embodiments, this initial gene group score may be calculated using RNA expression levels of at least three genes (e.g., at least 3, 4, 5, 6, 7, 8, 9, 10, or 11) in the invasion and angiogenesis inhibition FL group, which is defined by its constituent genes: THBS1, THBS2, THBS3, THBS4, COL18A1, SERPINF1, VASH1, MMRN2, TIMP2, TIMP3, and TIMP1.

In some embodiments, initial BC gene group scores comprise a gene group score for the lymphoma cell pro-survival signal group. In some embodiments, this initial gene group score may be calculated using RNA expression levels of at least three genes (e.g., at least 3, 4, 5, 6, 7, 8, or 9) in the lymphoma cell pro-survival signal group, which is defined by its constituent genes: TNFSF13B, TNFSF13, IL15, IL10, VEGFA, IL32, CD40LG, IL21 and IL33.

In some embodiments, initial BC gene group scores comprise a gene group score for the cancer promoting inflammation group. In some embodiments, this initial gene group score may be calculated using RNA expression levels of at least three genes (e.g., at least 3, 4, 5, 6, 7, 8, 9, 10, 11, 12, 13, or 14) in the cancer promoting inflammation group, which is defined by its constituent genes: IL6, IL21, EBI3, TGFB1, CXCL12, CSF1, CSF2, VEGFA, IDO1, S100A8, S100A9, CCL2, CCL5, and CXCL8.

In some embodiments, initial BC gene group scores comprise a gene group score for the cancer promoting inflammation FL group. In some embodiments, this initial gene group score may be calculated using RNA expression levels of at least three genes (e.g., at least 3, 4, 5, 6, 7, 8, or 9) in the cancer promoting inflammation FL group, which is defined by its constituent genes: IL10, IL21, EBI3, TGFB1, CSF1, VEGFA, IDO1, CCL2, and CCL5.

In some embodiments, initial BC gene group scores comprise a gene group score for the promotion of lymphoma dissemination group. In some embodiments, this initial gene group score may be calculated using RNA expression levels of at least three genes (e.g., at least 3, 4, 5, 6, 7, 8, or 9) in the promotion of lymphoma dissemination group, which is defined by its constituent genes: CXCL13, FGF2, TGFB1, MMP9, CXCL10, HGF, S100A11, IFI30, and HK3.

In some embodiments, initial BC gene group scores comprise a gene group score for the Treg recruitment and function group. In some embodiments, this initial gene group score may be calculated using RNA expression levels of at least three genes (e.g., at least 3, 4, or 5) in the promotion of Treg recruitment and function group, which is defined by its constituent genes: CCL17, CCL22, LGALS9, CD40LG, and CXCL13.

In some embodiments, initial BC gene group scores comprise a gene group score for the immunosuppressive factors group. In some embodiments, this initial gene group score may be calculated using RNA expression levels of at least three genes (e.g., at least 3, 4, 5, 6, 7, 8, 9, 10, 11, or 12) in the promotion of immunosuppressive factors group, which is defined by its constituent genes: LGALS1, TGFB3, IDO1, PTGES, TGFB1, CCL2, CXCL2, CXCL5, CCL22, EBI3, LGALS9, and CSF1.

In some embodiments, initial BC gene group scores comprise a gene group score for the immunosuppressive factors FL group. In some embodiments, this initial gene group score may be calculated using RNA expression levels of at least three genes (e.g., at least 3, 4, 5, 6, or 7) in the promotion of immunosuppressive factors FL group, which is defined by its constituent genes: LGALS1, TGFB3, IDO1, IL10, TGFB1, EBI3, and CSF1.

In some embodiments, initial BC gene group scores comprise a gene group score for the M2 polarization and response group. In some embodiments, this initial gene group score may be calculated using RNA expression levels of at least three genes (e.g., at least 3, 4, 5, 6, 7, 8, or 9) in the promotion of M2 polarization and response group, which is defined by its constituent genes: IL21, TGFB1, IL33, CSF2, LIF, TGFB3, TNFSF13B, TNFSF13, and TGFB3.

In some embodiments, initial BC gene group scores comprise a gene group score for the M2 polarization and response FL group. In some embodiments, this initial gene group score may be calculated using RNA expression levels of at least three genes (e.g., at least 3, 4, 5, 6, 7, 8, or 9) in the promotion of M2 polarization and response FL group, which is defined by its constituent genes: IL10, IL21, IL33, CSF2, LIF, TGFB3, IL4, TNFSF13B, and TNFSF13.

In some embodiments, initial BC gene group scores comprise a gene group score for the stroma and angiogenesis activation group. In some embodiments, this initial gene group score may be calculated using RNA expression levels of at least three genes (e.g., at least 3, 4, 5, 6, 7, 8, 9, 10, 11, 12, or 13) in the promotion of stroma and angiogenesis activation group, which is defined by its constituent genes: IL6, IL1B, VEGFA, PIGF, PGF, VEGFC, FGF2, HGF, PDGFA, PDGFB, SPP1, ANGPT1, and LTA.

In some embodiments, initial BC gene group scores comprise a gene group score for the stroma and angiogenesis activation FL group. In some embodiments, this initial gene group score may be calculated using RNA expression levels of at least three genes (e.g., at least 3, 4, 5, 6, 7, 8, 9, 10, or 11) in the promotion of stroma and angiogenesis activation FL group, which is defined by its constituent genes: IL1B, VEGFA, PGF, VEGFC, FGF2, HGF, PDGFA, PDGFB, SPP1, ANGPT1, and LTA.

In some embodiments, initial BC gene group scores comprise a gene group score for the pro-inflammatory cytokines group. In some embodiments, this initial gene group score may be calculated using RNA expression levels of at least three genes (e.g., at least 3, 4, 5, 6, 7, 8, 9, 10, 11, 12, or 13) in the promotion of pro-inflammatory cytokines group, which is defined by its constituent genes: IL15, CCL5, CCL2, CCL3, CXCL8, TNF, IL6, IFNG, IL1B, CXCL1, CXCL2, CXCL9, and CXCL10.

In some embodiments, initial BC gene group scores comprise a gene group score for the pro-inflammatory cytokines FL group. In some embodiments, this initial gene group score may be calculated using RNA expression levels of at least three genes (e.g., at least 3, 4, 5, 6, 7, 8, 9, 10, 11, or 12) in the promotion of pro-inflammatory cytokines FL group, which is defined by its constituent genes: IL15, CCL5, CCL2, CCL3, CXCL8, TNF, IFNG, IL1B, CXCL1, CXCL2, CXCL9, and CXCL10.

In some embodiments, initial BC gene group scores comprise a gene group score for the myeloid cell recruitment group. In some embodiments, this initial gene group score may be calculated using RNA expression levels of at least three genes (e.g., at least 3, 4, 5, 6, 7, or 8) in the promotion of myeloid cell recruitment group, which is defined by its constituent genes: CXCL8, CCL2, CSF1, CCL8, CXCL12, HBEGF, S100A9, and S100A8.

In some embodiments, initial BC gene group scores comprise a gene group score for the myeloid cell recruitment FL group. In some embodiments, this initial gene group score may be calculated using RNA expression levels of at least three genes (e.g., at least 3, 4, 5, or 6) in the promotion of myeloid cell recruitment FL group, which is defined by its constituent genes: CXCL8, CCL2, CSF1, CCL8, HBEGF, and S100A9.

In some embodiments, determining a cytokine signature comprises determining a respective gene group score for each of at least two of the following gene groups, using, for a particular gene group, RNA expression levels for at least three genes in the particular gene group to determine the gene group score for the particular group, the gene groups including: Type 1 response groups (e.g., CTL and Th1 cells activation group, M1 polarization group, TLS formation group), tumor suppression groups (e.g., tumor growth arrest group, metastasis inhibition group, angiogenesis inhibition group), B cell function groups (e.g., B cell activation group), immune cell recruitment groups (e.g., lymphocyte recruitment group, macrophage and DC recruitment group), myeloid inflammation groups (e.g., pro-inflammatory cytokines group, neutrophil recruitment and activation group), Type 2 response groups (e.g., Th2 response group, M2 polarization group, eosinophil/basophil recruitment group, eosinophil/basophil activation group), immune suppression groups (e.g., stromal suppressive factors group, myeloid suppressive factors group, CTL exclusion group, Treg polarization group), tumor promoting groups (e.g., tumor growth promotion group, induction of EMT group, metastasis promotion group), and/or stromal activation groups (e.g., angiogenesis induction group, CAF recruitment group).

In some embodiments, determining a cytokine signature comprises determining a respective gene group score for each of at least two of the following gene groups, using, for a particular gene group, RNA expression levels for at least three genes in the particular gene group to determine the gene group score for the particular group, the gene groups including: Type 1 response groups (e.g., T cell recruitment group, cancer inhibiting inflammation group, Th1/M1 polarization and response group, Th1/M1 polarization and response FL group), tumor suppression groups (e.g., invasion and angiogenesis inhibition group, invasion and angiogenesis inhibition FL group), tumor promotion groups (e.g., lymphoma cell pro-survival group, cancer promoting inflammation group, cancer promoting inflammation FL group, promotion of lymphoma and dissemination group), immune suppression groups (e.g., Treg recruitment and function group, immunosuppressive factors group, immunosuppressive factors FL group, M2 polarization and response group, M2 polarization and response FL group), stroma activation groups (e.g., stroma and angiogenesis activation group, stroma and angiogenesis activation FL group), pro-inflammatory cytokines group, pro-inflammatory cytokines FL group, myeloid cell recruitment group, and/or myeloid cell recruitment FL group.

As described above, aspects of the disclosure relate to determining a cytokine signature for a subject. That signature may include gene group scores (e.g., gene group scores generated using RNA expression data for gene groups listed in Table 1 or Table 2). Aspects of determining of cytokine signatures is described next with reference to FIG. 4.

In some embodiments, a cytokine signature comprises gene group scores generated using a gene set enrichment analysis (GSEA) technique to determine a gene group score for one or more (e.g., 1, 2, 3, 4, 5, 6, 7, 8, 9, 10, 11, 12, 13, 14, 15, 16, 17, 18, 19, 20, 21, 22, 23, 24, 25, 26, 27, or 28) gene groups listed in Table 1 or Table 2. In some embodiments, a cytokine signature comprises gene group scores generated using a gene set enrichment analysis (GSEA) technique to determine a gene group score for three or more (e.g., 3, 4, 5, 6, 7, 8, 9, 10, 11, 12, 13, 14, 15, 16, 17, 18, 19, 20, 21, 22, 23, 24, 25, 26, 27, or 28) gene groups listed in Table 1 or Table 2.

In some embodiments, each gene group score is generated using a gene set enrichment analysis (GSEA) technique, using RNA expression levels of at least some genes in the gene group. In some embodiments, using a GSEA technique comprises using single-sample GSEA. Aspects of single sample GSEA (ssGSEA) are described in Barbie et al. Nature. 2009 Nov. 5; 462(7269): 108-112, the entire contents of which are incorporated by reference herein. In some embodiments, ssGSEA is performed according to the following formula:

$$ssGSEA score = \frac{{{\sum }^{N}}_{i} {r}_{i}^{1.25}}{{{\sum }^{N}}_{i} {r}_{i}^{0.25}} - \frac{( M - N + 1 )}{2}$$

where rᵢ represents the rank of the ith gene in expression matrix, where N represents the number of genes in the gene set (e.g., the number of genes in the first gene group when ssGSEA is being used to determine a gene group score for the first gene group using expression levels of the genes in the first gene group), and where M represents total number of genes in expression matrix. Additional, suitable techniques of performing GSEA are known in the art and are contemplated for use in the methods described herein without limitation. In some embodiments, a cytokine signature is calculated by performing ssGSEA on expression data from a plurality of subjects, for example expression data from one or more cohorts of subjects, in order to produce a plurality of enrichment scores.

FIG. 4 depicts an illustrative example of how gene group scores may be determined as part of act 108 of process 100 (or act 208 of process 200). As shown in the example of FIG. 4, an initial STC or BC gene group scores or normalized STC or BC gene group scores comprise multiple gene group scores 420 (e.g., initial gene group scores) determined for respective multiple gene groups. Each gene group score, for a particular gene group, is computed by performing GSEA 410 (e.g., using ssGSEA) on RNA expression data for one or more (e.g., at least two, at least three, at least four, at least five, at least six, etc., or all) genes in the particular gene group 400.

For example, as shown in FIG. 4, a gene group score (labelled “Gene Group Score 1”) for gene group 1 (e.g., the M1 polarization group) is computed from RNA expression data for one or more genes in gene group 1. As another example, a gene group score (labelled “Gene Group Score 2”) for gene group 2 (e.g., the TLS formation group) is computed from RNA expression data for one or more genes in gene group 2. As another example, a gene group score (labelled “Gene Group Score 3”) for gene group 3 (e.g., the B cell activation group) is computed from RNA expression data for one or more genes in gene group 3. As another example, a gene group score (labelled “Gene Group Score 4”) for gene group 4 (e.g., the pro-inflammatory cytokines group) is computed from RNA expression data for one or more genes in gene group 4. As another example, a gene group score (labelled “Gene Group Score 5”) for gene group 5 (e.g., the Th2 response group) is computed from RNA expression data for one or more genes in gene group 5. As another example, a gene group score (labelled “Gene Group Score 6”) for gene group 6 (e.g., the M2 polarization group) is computed from RNA expression data for one or more genes in gene group 6. As another example, a gene group score (labelled “Gene Group Score 7”) for gene group 7 (e.g., the stromal suppressive factors group) is computed from RNA expression data for one or more genes in gene group 7. As another example, a gene group score (labelled “Gene Group Score 8”) for gene group 8 (e.g., the induction of EMT group) is computed from RNA expression data for one or more genes in gene group 8.

Although the example of FIG. 4 shows that the initial gene group scores (or normalized gene group scores) include eight gene group scores for a respective set of eight gene groups, it should be appreciated that in other embodiments, the cytokine signature may include scores for any suitable number of gene groups (e.g., not just 8; the number of groups could be fewer or greater than 8). As indicated by the vertical ellipsis in FIG. 4, determining gene group scores of a cytokine signature may comprise determining gene group scores for 9, 10, 11, 12, 13, 14, 15, 16, 17, 18, 19, 20, or more gene groups using RNA expression data from one or more respective genes in each respective gene group, as aspects of the technology described herein are not limited in this respect. In another example, a cytokine signature may include scores for only a subset of the gene groups listed in Table 1 or Table 2. As another example, the gene group score may include one or more scores for one or more gene groups other than those gene groups listed in Table 1 or Table 2 (either in addition to the score(s) for the groups in Table 1 or Table 2, or instead of one or more of the scores for the groups in Table 1 or Table 2).

In some embodiments, RNA expression levels for a particular gene group may be embodied in at least one data structure having fields storing the expression levels. The data structure or data structures may be provided as input to software comprising code that implements a GSEA technique (e.g., the ssGSEA technique) and processes the expression levels in the at least one data structure to compute a score for the particular gene group.

The number of genes in a gene group used to determine a gene group score may vary. In some embodiments, all RNA expression levels for all genes in a particular gene group may be used to determine a gene group score for the particular gene group. In other embodiments, RNA expression data for fewer than all genes may be used (e.g., RNA expression levels for at least two genes, at least three genes, at least five genes, between 2 and 10 genes, between 5 and 15 genes, or any other suitable range within these ranges).

In some embodiments, RNA expression levels for a particular gene group may be embodied in at least one data structure having fields storing the expression levels. The data structure or data structures may be provided as input to software comprising code that is configured to perform suitable scaling (e.g., median scaling) to produce a score for the particular gene group.

In some embodiments, ssGSEA is performed on expression data comprising three or more (e.g., 3, 4, 5, 6, 7, 8, 9, 10, 11, 12, 13, 14, 15, 16, 17, 18, 19, or 20) gene groups set forth in Table 1 or Table 2. In some embodiments, each of the gene groups separately comprises one or more (e.g., 1, 2, 3, 4, 5, 6, 7, 8, 9, 10, 11, 12, 13, 14, 15, 16, 17, 18, 19, 20, or more) genes listed in Table 1 or Table 2. In some embodiments, a cytokine signature is produced by performing ssGSEA on all of the gene groups in Table 1, each gene group including all listed genes in Table 1. In some embodiments, one or more (e.g., a plurality) of gene group scores are normalized in order to produce a cytokine signature for the expression data (e.g., expression data of the subject or of a cohort of subjects). In some embodiments, the initial STC or BC gene group scores are normalized by median scaling prior to being normalized to the gene group scores for a cohort of subject having the same type of cancer. In some embodiments, the initial gene group scores are normalized by rank estimation and median scaling prior to being normalized to the gene group scores for a cohort of subject having the same type of cancer.

Some aspects of determining gene group scores for gene groups are also described in U.S. Patent Publication No. 2020-0273543, entitled “SYSTEMS AND METHODS FOR GENERATING, VISUALIZING AND CLASSIFYING MOLECULAR FUNCTIONAL PROFILES”, the entire contents of which are incorporated by reference herein.

### Therapeutic Indications

Aspects of the disclosure relate to methods of identifying or selecting a therapeutic agent for a subject based upon determination of the subject's cytokine signature. The disclosure is based, in part, on the recognition that subjects having certain cytokine signatures have an increased likelihood of responding to certain therapies (e.g., immunotherapeutic agents, tyrosine kinase inhibitors (TKIs), etc.) relative to subjects having other cytokine signatures.

In some embodiments, the present disclosure provides methods for identifying a subject having, suspected of having, or at risk of having a certain cancer (e.g., an STC or BC) as having an increased likelihood of having a good prognosis (e.g., as measured by overall survival (OS) or progression-free survival (PFS). In some embodiments, the method comprises determining a cytokine signature of the subject as described herein.

In some embodiments, the methods comprise identifying the subject as having a decreased risk of cancer progression relative to subjects having other cytokine signatures. In some embodiments, “decreased risk of cancer progression” may indicate better prognosis of cancer or decreased likelihood of having advanced disease in a subject. In some embodiments, “decreased risk of cancer progression” may indicate that the subject who has cancer is expected to be more responsive to certain treatments. For instance, “decreased risk of cancer progression” indicates that a subject is at least 10%, 20%, 30%, 40%, 50%, 60%, 70%, 80%, 90%, or 100% likely to experience a progression-free survival event (e.g., relapse, retreatment, or death) than another cancer patient or population of cancer patients (e.g., patients having the same cancer, but not the same cytokine signature as the subject).

In some embodiments, the methods further comprise identifying the subject as having an increased risk of cancer progression relative to subjects having other cytokine signatures. In some embodiments, “increased risk of cancer progression” may indicate less positive prognosis of cancer or increased likelihood of having advanced disease in a subject. In some embodiments, “increased risk of cancer progression” may indicate that the subject who has cancer is expected to be less responsive or unresponsive to certain treatments and show less or no improvements of disease symptoms. For instance, “increased risk of cancer progression” indicates that a subject is at least 10%, 20%, 30%, 40%, 50%, 60%, 70%, 80%, 90%, or 100% more likely to experience a progression-free survival event (e.g., relapse, retreatment, or death) than another cancer patient or population of cancer patients (e.g., patients having the same type of cancer, but not the same cytokine signature as the subject).

In some embodiments, the methods described herein comprise the use of at least one computer hardware processor to perform the determination.

In some embodiments, the present disclosure provides a method for providing a prognosis, predicting survival, or stratifying patient risk of a subject suspected of having, or at risk of having cancer. In some embodiments, the method comprises determining a cytokine signature of the subject as described herein.

In some embodiments, methods described by the disclosure further comprise a step of administering one or more therapeutic agents to the subject based upon the determination of the subject's cytokine signature. In some embodiments, a subject is administered one or more (e.g., 1, 2, 3, 4, 5, or more) immuno-oncology (IO) agents. An IO agent may be a small molecule, peptide, protein (e.g., antibody, such as monoclonal antibody), interfering nucleic acid, or a combination of any of the foregoing. In some embodiments, the IO agents comprise a PD1 inhibitor, PD-L1 inhibitor, or PD-L2 inhibitor. Examples of IO agents include but are not limited to cemiplimab, nivolumab, pembrolizumab, avelumab, durvalumab, atezolizumab, BMS1166, BMS202, etc. In some embodiments, the IO agents comprise a combination of atezolizumab and albumin-bound paclitaxel, pembrolizumab and albumin-bound paclitaxel, pembrolizumab and paclitaxel, or pembrolizumab and Gemcitabine and Carboplatin.

In some embodiments, a subject is administered one or more (e.g., 1, 2, 3, 4, 5, or more) tyrosine kinase inhibitors (TKIs). A TKI may be a small molecule, peptide, protein (e.g., antibody, such as monoclonal antibody), interfering nucleic acid, or a combination of any of the foregoing. Examples of TKIs include but are not limited to Axitinib (Inlyta®), Cabozantinib (Cabometyx®), Imatinib mesylate (Gleevec®), Dasatinib (Sprycel®), Nilotinib (Tasigna®), Bosutinib (Bosulif®), Sunitinib (Sutent®), etc. In some embodiments, the TKI inhibitor comprises neratinib, apatinib, toripalimab and anlotinib, or anlotinib.

In some embodiments, a subject is administered an effective amount of a therapeutic agent. “An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons, or for virtually any other reasons.

Empirical considerations, such as the half-life of a therapeutic compound, generally contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally (but not necessarily) based on treatment, and/or suppression, and/or amelioration, and/or delay of a cancer. Alternatively, sustained continuous release formulations of an anti-cancer therapeutic agent may be appropriate. Various formulations and devices for achieving sustained release are known in the art.

In some embodiments, dosages for an anti-cancer therapeutic agent as described herein may be determined empirically in individuals who have been administered one or more doses of the anti-cancer therapeutic agent. Individuals may be administered incremental dosages of the anti-cancer therapeutic agent. To assess efficacy of an administered anti-cancer therapeutic agent, one or more aspects of a cancer (e.g., cytokine signature, tumor microenvironment, tumor formation, tumor growth, or TME types, etc.) may be analyzed.

As used herein, the term “treating” refers to the application or administration of a composition including one or more active agents to a subject, who has a cancer, a symptom of a cancer, or a predisposition toward a cancer, with the purpose to cure, heal, alleviate, relieve, alter, remedy, ameliorate, improve, or affect the cancer or one or more symptoms of cancer, or the predisposition toward cancer.

Alleviating cancer includes delaying the development or progression of the disease, or reducing disease severity. Alleviating the disease does not necessarily require curative results. As used therein, “delaying” the development of a disease (e.g., a cancer) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detected and assessed using clinical techniques known in the art. Alternatively, or in addition to the clinical techniques known in the art, development of the disease may be detectable and assessed based on other criteria. However, development also refers to progression that may be undetectable. For purpose of this disclosure, development or progression refers to the biological course of the symptoms. “Development” includes occurrence, recurrence, and onset. As used herein “onset” or “occurrence” of a cancer includes initial onset and/or recurrence.

### Computer Implementation

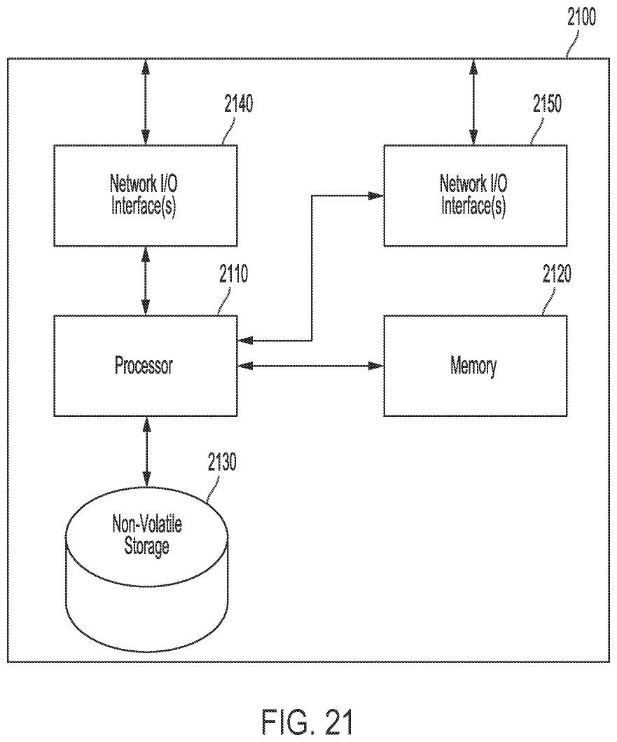

An illustrative implementation of a computer system 2100 that may be used in connection with any of the embodiments of the technology described herein (e.g., such as the method of FIG. 1, FIG. 2, FIG. 3, FIG. 4, etc.) is shown in FIG. 21. The computer system 2100 includes one or more processors 2110 and one or more articles of manufacture that comprise non-transitory computer-readable storage media (e.g., memory 2120 and one or more non-volatile storage media 2130). The processor 2110 may control writing data to and reading data from the memory 2120 and the non-volatile storage device 2130 in any suitable manner, as the aspects of the technology described herein are not limited to any particular techniques for writing or reading data. To perform any of the functionality described herein, the processor 2110 may execute one or more processor-executable instructions stored in one or more non-transitory computer-readable storage media (e.g., the memory 2120), which may serve as non-transitory computer-readable storage media storing processor-executable instructions for execution by the processor 2110.

Computing device 2100 may also include a network input/output (I/O) interface 2140 via which the computing device may communicate with other computing devices (e.g., over a network), and may also include one or more user I/O interfaces 2150, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

TABLE 3
Genes for Tables 1 and 2:

| Gene | Accession Numbers |
|---|---|
| IFNG | NM_000619 |
| IL18 | NM_001386420; XM_011542805; NM_001562; ; NM_001243211 |
| IL15RA | XM_011519471; XM_047425181; NM_002189; NR_046362; XM_011519463; |
| | XM_011519465; XM_011519468; XM_011519469; XM_017016197; |
| | XM_047425182; XM_047425184; XM_011519467; XM_017016198; |
| | XM_017016199; NM_001351095; XM_011519462; XM_011519464; |
| | XM_011519466; XM_047425185; XM_047425190; XM_011519475; |
| | XM_017016195; XM_047425187; XM_047425191; XM_047425189; |
| | NM_001243539; NM_001351096; NM_001351097; NM_172200; |
| | XM_011519470; XM_047425186; NM_001256765; XM_011519461; |
| | XM_011519474; XM_011519477; XM_047425183; XM_011519472; |
| | XM_011519476; XM_047425188 |
| TNF | NM_000594 |
| IL27 | NM_145659 |
| CCL5 | NM_001278736; NM_002985; |
| TNFSF11 | XM_017020803; XM_047430707; NM_033012; XM_011535280; |
| | XM_017020802; ; NM_003701 |
| CD40LG | NM_000074; |
| FLT3LG | XM_047438520; XM_047438522; NM_001204503; NM_001278637; |
| | NM_001278638; XM_011526678; XM_047438521; XM_011526677; |
| | XM_047438523; XM_006723116; XM_011526675; XM_011526680; |
| | XM_047438519; XM_011526676; NM_001204502; NM_001459 |
| TNFSF9 | NM_003811 |
| CD70 | NM_001252; NM_001330332 |
| CCL21 | NM_002989 |
| CSF2 | NM_000758; |
| IL23A | NM_016584 |
| IL12B | NM_002187 |
| CXCL11 | NM_001302123; NM_005409 |
| CCL18 | NM_002988 |
| CXCL10 | NM_001565; NR_168520 |
| CXCL9 | NM_002416 |
| CCL2 | NM_002982; |
| CXCL13 | NM_001371558; NM_006419 |
| CCL8 | NM_005623 |
| CCL4 | NM_002984 |
| CCL3 | NR_168496; ; NR_168495; NM_002983; NR_168494 |
| CCL19 | NM_006274 |
| CXCR2 | XM_047444190; XM_047444188; NM_001168298; NM_001557; |
| | XM_005246530; XM_047444189; XM_017003991; XM_047444191; |
| | XM_047444187; |
| ACKR2 | NM_001296 |
| ACKR4 | NM_016557; NM_178445 |
| ACKR1 | NM_002036; ; NM_001122951 |
| BAMBI | NM_012342 |
| TNFSF10 | NR_033994; NM_001190943; NM_003810; NM_001190942 |
| LTA | NM_000595; NM_001159740; XM_047418773; |
| CCL28 | NM_001301874; NM_001301873; NM_019846; XR_007058611; XR_007058613; |
| | XR_925633; NM_148672; NM_001301875; XR_007058610; XR_007058612; |
| | XR_427660; XR_241706 |
| IL12A | NM_000882; NM_001354583; NM_001354582; NM_001397992 |
| CXCR3 | XM_017029435; ; XM_017029436; XM_047442010; NM_001504; |
| | NM_001142797; XM_005262256; XM_005262257 |
| CCR5 | NM_001100168; ; NM_001394783; NM_000579 |
| ARRB2 | XM_047436065; NM_001257328; NM_001257331; NR_047516; |
| | XM_047436067; NM_001257329; XM_047436066; NM_001330064; |
| | NM_001257330; XM_017024645; XM_024450752; XM_047436062; |
| | XM_047436064; NM_004313; XM_047436063; NM_199004; XM_024450753; |
| | XM_011523858 |
| TNFSF15 | NM_001204344; NM_005118; |
| IL4 | NM_000589; ; NM_001354990; NM_172348 |
| IL10 | NM_000572; ; NR_168467; NR_168466; NM_001382624 |
| TNFSF13B | NM_006573; XM_047430055; NM_001145645; |
| TNFSF13 | NM_001198622; NM_172088; NR_073490; NM_001198624; NM_003808; |
| | NM_172087; ; NM_001198623 |
| CCL20 | NM_001130046; NM_004591 |
| CCL17 | XM_047434448; XM_011523256; NM_002987; XM_017023530 |
| CCL22 | XM_047434450; XM_047434449; NM_002990 |
| TGFB1 | NM_000660; XM_011527242 |
| CSF1 | NM_000757; NM_172210; ; XM_017000369; XM_047446752; NM_172211; |
| | NM_172212 |
| XCL1 | NM_002995 |
| CXCL12 | NM_000609; NM_001277990; NM_199168; NM_001178134; NM_001033886; |
| IL1A | NM_001371554; NM_000575 |
| IL1B | NM_000576; XM_047444175 |
| IL6 | NM_001318095; NM_000600; NM_001371096; XM_005249745; |
| LIF | XM_047441361; NM_001257135; ; NM_002309 |
| CXCL2 | NM_002089 |
| CXCL1 | NM_001511; NR_046035 |
| CXCL3 | NM_002090 |
| PF4 | NM_002619; ; NM_001363352 |
| CXCL5 | NM_002994 |
| CXCL8 | NM_000584; ; NM_001354840 |
| CSF3 | NR_168489; NR_168491; NM_000759; NM_001178147; NM_172219; |
| | NM_172220; NR_168490; NR_033662 |
| PPBP | NM_002704 |
| CXCL6 | NM_002993 |
| IL11 | NM_000641; NM_001267718 |
| IL13 | NM_001354992; NM_001354991; NM_002188; NM_001354993; |
| TSLP | NM_138551; XM_011543698; XM_047417847; NM_033035; NR_045089; |
| | XM_047417846 |
| CCL13 | NM_005408 |
| IL33 | XM_047424061; XM_047424063; NM_001314046; NM_001314048; |
| | XM_047424062; NM_001314047; NM_001353802; NM_033439; |
| | XM_047424064; ; NM_001199641; NM_001314045; XM_047424060; |
| | NM_001199640; XM_017015285; NM_001314044 |
| CCL24 | XM_011516460; NM_002991; NM_001371193 |
| TGFB2 | NM_003238; NR_138149; NR_138148; ; NM_001135599 |
| TGFB3 | NM_001329938; NM_003239; ; NM_001329939 |
| CCL11 | NM_002986 |
| CCL26 | NM_006072; ; NM_001371936; NM_001371938 |
| ENPP3 | NR_133007; NM_005021; XM_017010932; XM_011535897 |
| IL1RL1 | XM_006712839; NM_016232; NM_173459; NM_003856; XM_011512151; |
| | NR_104167; NM_001282408 |
| IL5RA | NM_175726; NM_175724; NM_175727; XM_011533677; NM_001243099; ; |
| | XM_011533678; NM_175725; NM_175728; NM_000564 |
| ANGPT1 | NM_001314051; ; NM_001146; NM_001199859; XM_047421699; NM_139290 |
| TDO2 | NM_005651 |
| TGFBI | NM_000358; |
| FGL2 | NM_006682 |
| IDO1 | NM_002164; |
| EBI3 | NM_005755 |
| IL4I1 | NM_001385639; ; NM_172374; NM_152899; NR_047577; NM_001258018; |
| | NM_001258017 |
| EDNRB | NM_001122659; NM_003991; NM_001201397; ; NM_000115; NR_047024 |
| PDGFC | XM_047415970; XM_017008455; NM_016205; XM_047415971; |
| | XM_047415969; XM_047415972; NR_036641 |
| FGF2 | NM_001361665; NM_002006 |
| GAS6 | NM_001143945; NM_001143946; NM_000820; |
| TNFAIP6 | NM_007115; XM_047445635 |
| VEGFA | NM_001171625; NM_003376; NM_001033756; NM_001171624; |
| | NM_001171626; NM_001171630; NM_001025366; ; NM_001317010; |
| | NM_001025368; NM_001025370; NM_001171623; NM_001171622; |
| | NM_001171628; NM_001171629; NM_001204385; NM_001025367; |
| | NM_001025369; NM_001171627; NM_001204384; NM_001287044 |
| AXL | NM_001699; NM_021913; NM_001278599 |
| IL2RA | NM_001308243; NM_001308242; NM_000417 |
| HGF | NM_000601; NM_001010933; XM_047420293; ; NM_001010934; |
| | NM_001010931; NM_001010932 |
| FGF7 | NM_002009 |
| FGF10 | NM_004465; XM_005248264 |
| IGF1 | XM_017019263; XM_017019261; XM_017019262; NM_001414007; |
| | NM_001414005; XM_017019259; NM_001414006; NM_001111284; |
| | NM_001111285; ; NM_001111283; NM_000618 |
| EGF | XM_005262796; XM_011531707; XM_017007848; XM_017007850; |
| | XM_047449723; NM_001178131; XM_047449725; XM_017007847; |
| | XM_017007855; XM_047449726; XM_047449727; XM_047449729; |
| | XM_017007854; NM_001963; XR_001741156; XM_017007845; XM_017007849; |
| | XM_047449728; ; NM_001178130; XM_017007846; XM_017007853; |
| | NM_001357021; XM_017007851; XM_047449724; XM_047449730 |
| CCL7 | NM_006273 |
| ARTN | NM_057160; XM_047433665; NM_057090; NM_001136215; XM_047433657; |
| | NM_057091; NM_003976 |
| OSM | NM_001319108; NM_020530; XM_047441387 |
| CCR4 | XM_017005687; NM_005508 |
| CCR6 | NM_001394582; NM_031409; NM_004367 |
| CXCR4 | NM_001348059; NM_001348060; XM_047445802; NM_001348056; |
| | NM_003467; NM_001008540 |
| HIF1A | NM_001530; NM_181054; ; NM_001243084 |
| VEGFC | NM_005429; |
| PDGFA | XM_011515415; ; XM_011515419; XM_011515418; NM_001395365; |
| | NR_172526; XM_011515416; XM_047420455; XM_047420458; NM_001395363; |
| | NM_001395364; NM_033023; XM_017012289; NM_001395366; |
| | XM_047420457; NR_172527; XM_047420456; NM_002607 |
| PDGFB | XM_047441393; ; NM_002608; XM_047441394; NM_033016 |
| TNFSF12 | NR_037146; NM_003809 |
| FGF1 | NM_001144892; NM_001257209; NM_001257212; NM_001354959; |
| | NM_001354962; NM_001354963; NM_000800; NM_001354956; |
| | NM_001354961; NM_001144935; NM_001354952; NM_001354954; |
| | NM_001354957; NM_001257206; NM_001257208; NM_001354951; |
| | NM_033137; NM_001144934; NM_001257211; NM_001354964; |
| | NM_001257207; NM_001354955; NM_033136; NM_001257210; |
| | NM_001354958; NM_001257205; NM_001354953 |
| MMP2 | NM_001302509; NM_001127891; NM_001302508; NM_001302510; |
| | NM_004530 |
| IL15 | NM_000585; ; NM_172175; NM_172174; NR_037840 |
| CX3CL1 | NM_001304392; NM_002996 |
| XCL2 | NM_003175 |
| THBS1 | NM_003246; XM_047432980; XM_011521971 |
| THBS2 | NM_003247; NM_001381940; NM_001381941; NM_001381939; NR_167745; |
| | NM_001381942; NR_167744 |
| THBS3 | XM_047429305; XM_047429306; NM_001407555; NM_001407562; |
| | XM_047429308; XM_047429324; NM_001407490; NM_007112; |
| | XM_006711498; XM_011509928; XM_011509932; NM_001252608; |
| | NM_001407556; NM_001407559; NM_001407561; XM_011509931; |
| | XM_047429292; NM_001407487; NM_001407554; NR_045553; |
| | XM_011509926; XM_011509929; XM_011509930; XM_047429322; |
| | NM_001252607; NM_001407488; NM_001407557; NM_001407560; |
| | NR_176392; XM_011509936; NM_001407558; XR_002957482; XM_011509927; |
| | XM_047429301; XM_047429323; XM_047429326; ; NR_176393 |
| THBS4 | XM_047417678; ; NM_001306214; NM_003248; NM_001306213; |
| | XM_017009799; NM_001306212; XR_007058633 |
| COL18A1 | NM_001379500; ; NM_130444; NM_130445; NM_030582 |
| SERPINF1 | XR_004837577; NM_001329904; ; NM_001329905; NM_002615; |
| | NM_001329903 |
| VASH1 | XM_047431085; XM_017021091; XM_047431083; XM_047431082; |
| | XM_047431084; XM_017021087; XM_047431081; NM_014909 |
| MMRN2 | NM_024756; |
| TIMP2 | NM_003255 |
| TIMP3 | NM_000362 |
| TIMP1 | NM_003254 |
| IL32 | NM_001012633; NM_001012718; NM_001012635; NM_001012632; ; |
| | NM_001012631; NM_001369589; NM_001369593; NM_001376923; |
| | NM_001369590; NM_001308078; NM_001369588; NM_001369592; |
| | NM_001369595; NM_004221; NM_001012636; NM_001369587; |
| | NM_001369591; NM_001012634; NM_001369596 |
| IL21 | NM_021803; ; NM_001207006 |
| S100A8 | NM_001319197; NM_001319198; NM_002964; NM_001319196; |
| | NM_001319201 |
| S100A9 | NM_002965 |
| MMP9 | NM_004994; |
| S100A11 | NM_005620 |
| IFI30 | NM_006332 |
| HK3 | XM_047417134; XM_011534540; ; NM_002115; XR_941102 |
| LGALS9 | XM_011524796; NM_001330163; NR_024043; XM_006721893; XM_006721895; |
| | NM_002308; XM_006721892; NM_009587 |
| LGALS1 | NM_002305 |
| PTGES | NM_198797; NM_004878 |
| PIGF | XM_005264369; NM_002643; XM_011532908; NM_173074 |
| PGF | NM_001293643; NM_002632; NM_001207012; XM_047431476 |
| SPP1 | NM_001251829; ; NM_001040060; NM_001251830; NM_001040058; |
| | NM_000582 |
| HBEGF | NM_001945 |

### EXAMPLES

The binding of a cytokine to its receptor can trigger multiple signaling cascades that result in altered cell function. In case of cancer, cytokines exhibit both immunomodulatory effects, influencing the tumor microenvironmental (TME) landscape and tumor-stimulating effects, and promoting tumor growth and stroma remodeling. For example, various cytokines activate the processes of angiogenesis for the germination of new vessels, and induce the migration of immune cells to the tumor site: subsets of B and T lymphocytes, tumor-associated macrophages (TAMs), and granulocytes. Cytokines can also have tumor-suppressive effects by inhibiting tumor growth and angiogenesis processes or by activating the anti-tumor immune response. Thus, by having a complete picture of cytokine gene expression and cytokine gene signature scores, a better understanding of the biology of the tumor and its development can be determined. As a result, it becomes possible to choose treatment options tailored to the specific cytokine gene signatures scores and/or cytokine gene expression because cytokines are expected to affect therapeutic efficacy.

The analysis of a patient tumor cytokine signatures and/or cytokine gene expression described herein was based on the transcriptional profiles of tumor biopsies. Tumor cytokine signatures display the expression intensity of cytokine gene groups related to different processes in the tumor microenvironment. The expression of single cytokine genes gives additional information about the cytokine landscape, and suggests a treatment approach if the gene is a biomarker.

One challenge in determining cancer therapeutics based on cytokines is the pleiotropic effects of different cytokines, and the pleiotropic effects of the same cytokine in different conditions. For example, homeostasis or inflammation, or hypoxia or normoxia could affect cytokine production by TME cells, and promote/induce diverse cell responses to cytokines such as apoptosis or survival signals. Moreover, cytokines could have more than one specific receptor, and cytokines from one family could induce signal transduction pathways through cognate receptors from the same family. Thus, cytokine gene expression signatures developed herein for solid and hematological malignancies were validated for their specificity to the process they are describing on the appropriate sample cohorts.

Assessment of cell phenotype or morphology, although it has a partial association with cell functional activity, doesn't give the full picture of the immune response development in a certain sample (e.g., a tumor). The use of a tumor cytokine gene signature or cytokine gene expression can allow for determining the direction and intensity of the immune cell responses, as well as to identify the main mechanisms of immunosuppression taking place in the tumor, which can help to determine the best strategy for treatment selection. At the moment, there exist several therapies aimed at specific cytokines; thus, the evaluation of the cytokine gene expressions will widen the spectrum of candidate therapies for a certain patients. Additionally, cytokine profiling can help guide development of new therapeutic approaches, for example, inducing re-polarization of macrophages (M2 to M1), or assessing the possible efficiency of the immune checkpoint inhibitors or dendritic cell vaccines through the overview of the immune system activation.

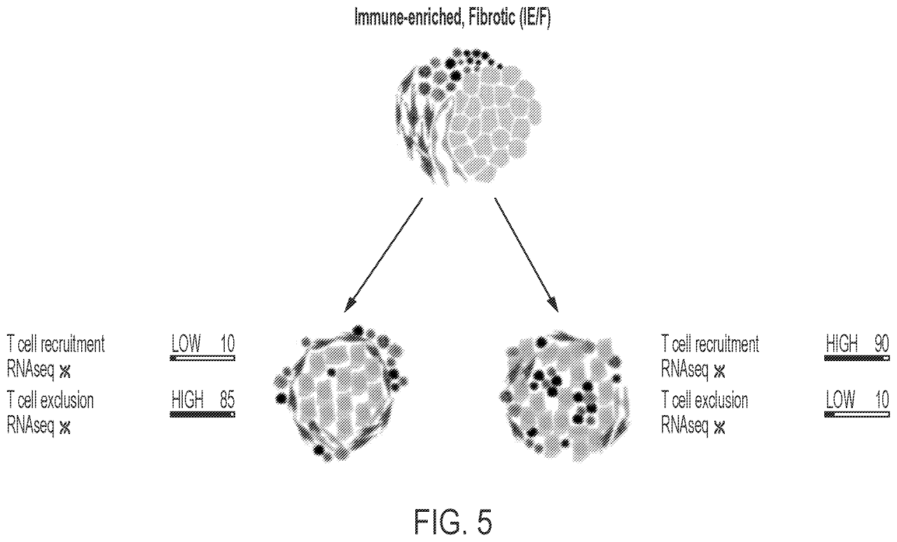

Additionally, while bulk RNA sequencing data does not provide information about spatial arrangement of the immune cells inside the tumor, cytokine signature scores may indirectly provide us with the missing knowledge about the tumor architecture (FIG. 5). For example, according to the bulk RNA data, a large number of cytotoxic T cells can be predicted to be present in the tumor. However, a cytokine signature (FIG. 5) shows the tumor has a high exclusion signature score and a low T cell recruitment signature score. Thus, these T cells are most likely lined up around the fibrous cap of the tumor, so there will be a low chance of checkpoint inhibitor therapy response. Alternatively, a high T cell recruitment signature score and a low exclusion signature score indicates that the patient is a better candidate for checkpoint inhibitor treatment.

### Tumor Cytokine Signature and Cytokine Gene Expression Structure

A tumor cytokine signature and cytokine gene expression was developed to describe the cytokine inflammatory environment of a sample (e.g., a tumor sample). The cytokine inflammatory environment of a sample can be illustrated using a solar diagram where the intensity of different cytokine-mediated biological processes, which are divided into specific functional groups, are visualized as rays (FIG. 6). All of the cytokine-mediated processes belong to one of the three large modules: Pro-tumor effects, Anti-tumor effects, or Other effects. The length and color intensity of each ray depends on the gene expression signature score level describing a certain cytokine-mediated process that was normalized on the cohort of patients with the same diagnosis (FIG. 6).

Cytokine gene groups included in the tumor cytokine signatures are listed in Table 1—for solid tumor cancers (STCs), and in Table 2—for hematological malignancies (blood cancers (BCs), for example, DLBCL and follicular lymphoma (FL)). Calculation of a signature score was performed using the single-sample gene set enrichment analysis (ssGSEA), which allows scoring samples based on the expression of genes comprising a signature. (Subramanian A et al. Proc Natl Acad Sci. 2005; 102(43):15545-15550. doi:10.1073/pnas.0506580102; and Bagaev A et al. Cancer Cell. 2021; 39(6):845-865.e7. doi:10.1016/j.ccell.2021.04.014). After that, the calculated ssGSEA scores of the patient's sample were normalized relative to a cohort of patients with the same diagnosis from the TCGA project (i.e., COAD cohort for a patient with colorectal cancer) and ranged from 0 to 100%. The smallest score of a signature or the smallest single gene expression in a reference cohort was considered a zero for this signature/single gene, the highest score or single gene expression—100%. Next, the normalized signature score/single gene expression of a patient was assigned with a High/Med/Low expression level according to the thresholds: less than 17% is Low, more than 83% is High, 17%-83% is Medium.

### Cytokine Signatures Development and Validation

Developing cytokine gene signatures involves several steps. First, based on literature analysis, genes encoding cytokines, growth factors and other signaling soluble molecules were chosen; these cytokine genes were chosen because they are reported to induce, take part in, or correlate with a certain biological process, like tumor progression or T cell activation. Further, the selected genes were grouped into functional gene expression signatures (FGES) (also referred to herein as “gene groups”) according to their biological contribution to anti-tumor immune response, tumor progression, or other immune processes they mediate (Table 1, Table 2). Next, the collected FGESs were subjected to a validation process in order to check signature technical and biological adequacy: stability, specificity, robustness, etc. Technical validation criteria included: 1) gene expression check—for each gene in a signature, gene expression should be greater than 1 transcript per million (tpm) for most of the analyzed patients. This resulted in selection of TCGA project samples and NCICCR cohort samples of Diffuse Large B-Cell Lymphoma (DLBCL), and MALY-DE (ICGC) cohort samples of Follicular lymphoma (FL); and 2) positive cross-correlation of the genes comprising the signature. According to biological criteria, FGES and single genes it consists of should: 1) differentiate a group of interest and have a minimal overlap with other unrelated signatures and molecular pathways; and 2) should be concordant with other models, i.e., cell deconvolution analysis. FGESs were validated on training data (75% of all samples), then finally tested on the test data (the remaining 25% of samples). During all the validation steps, genes that do not correspond to the above-mentioned criteria were eliminated from the signature, and new candidate genes could be added.

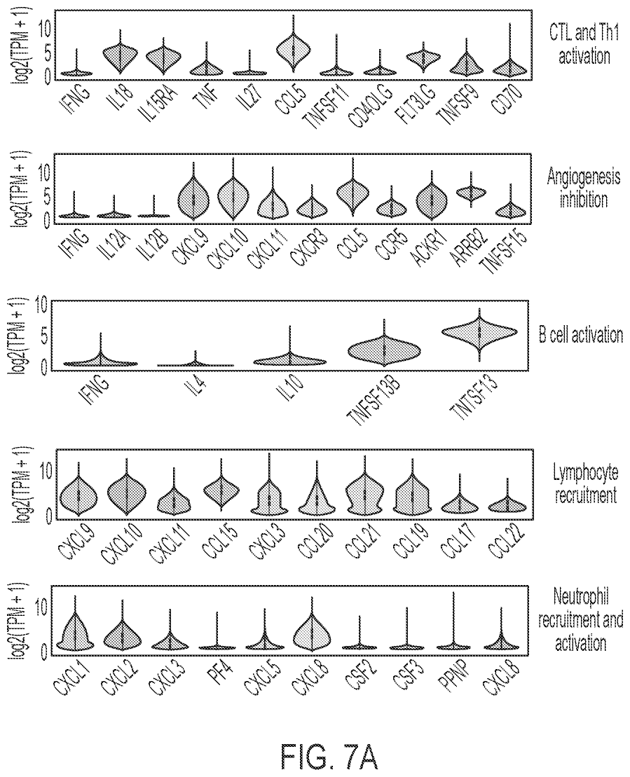

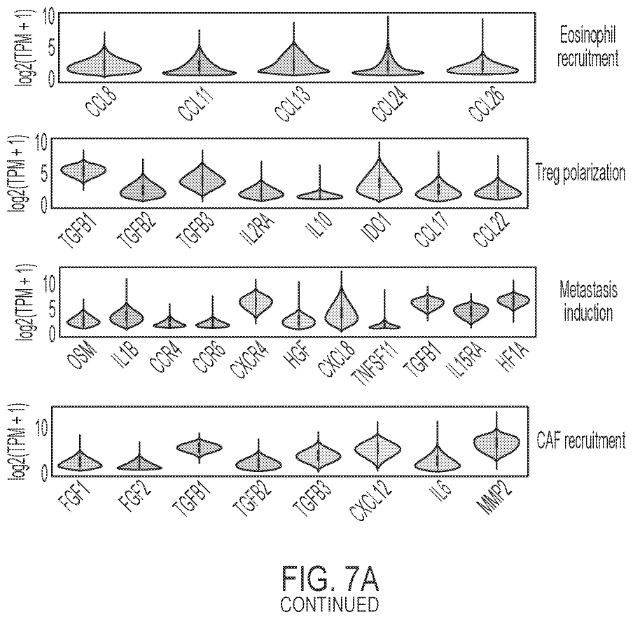

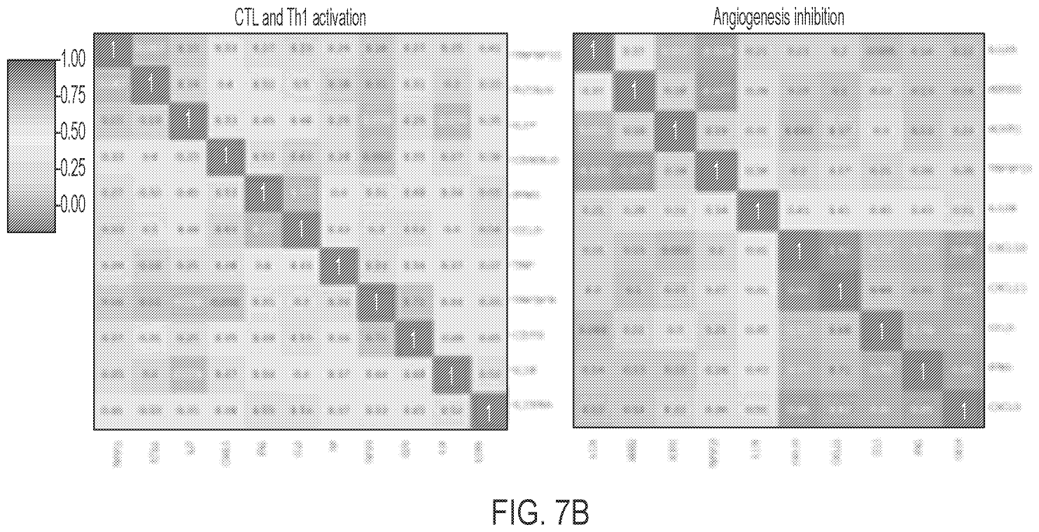

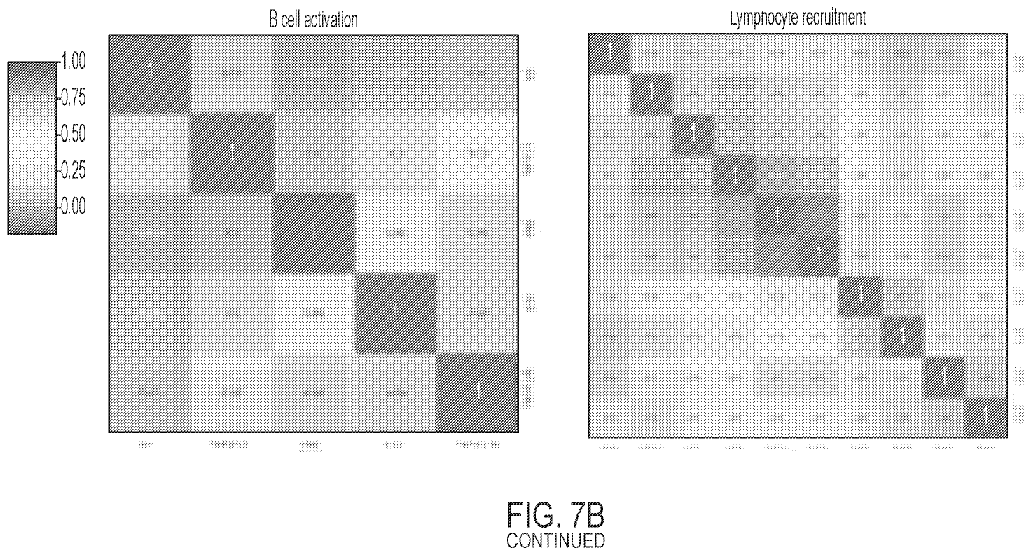

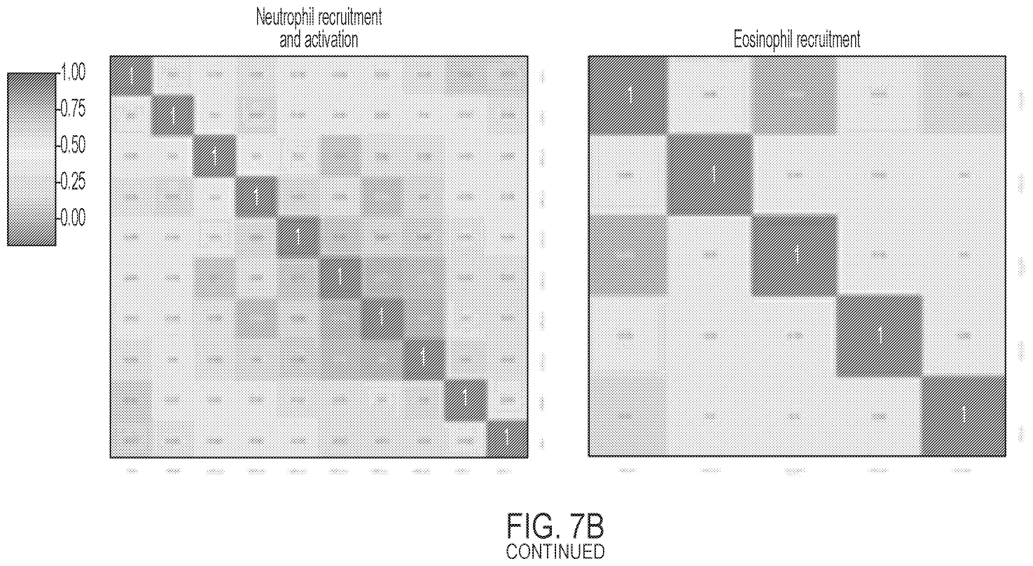

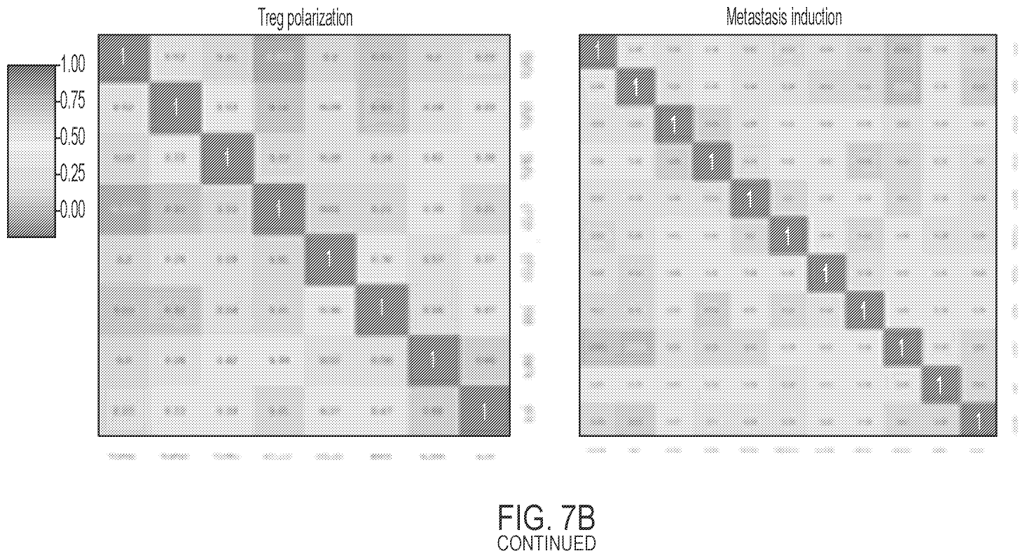

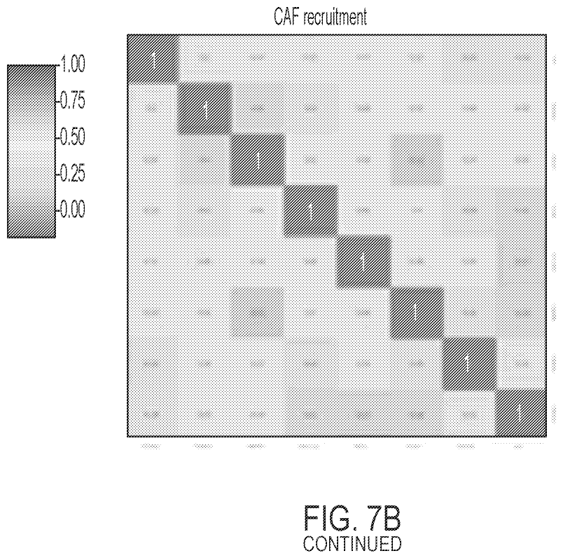

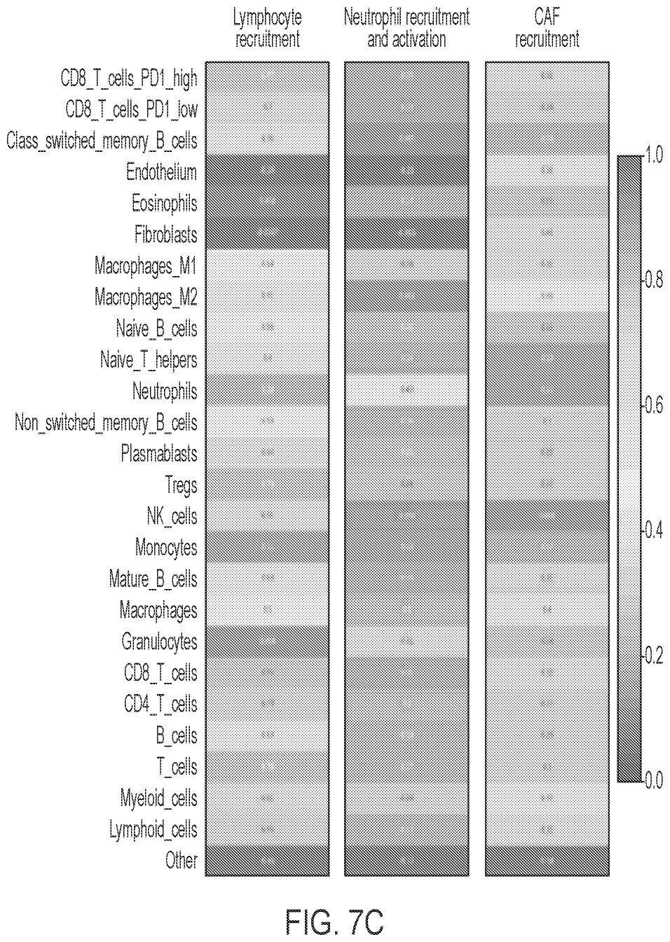

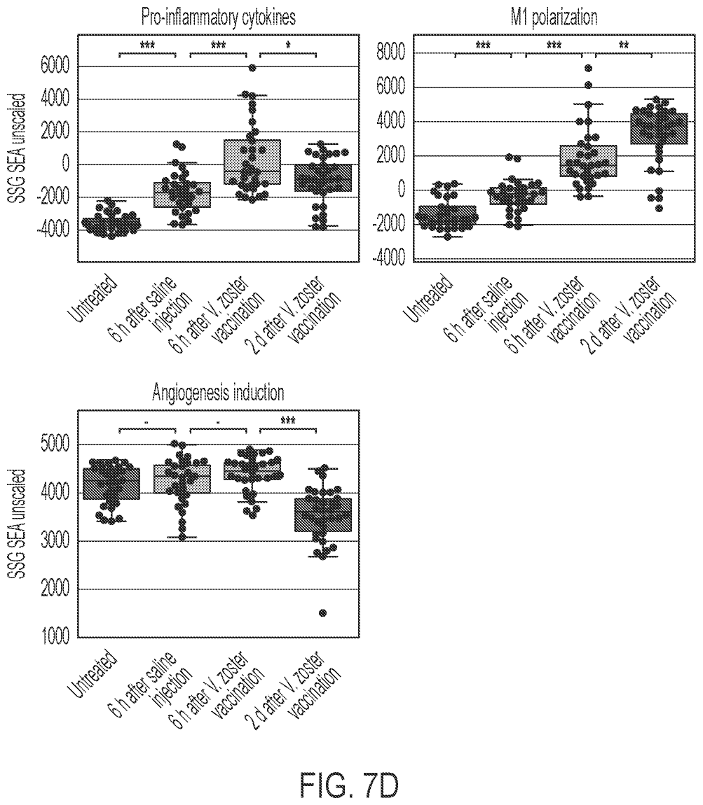

FIGS. 7A-7B present the results of validation of FGES developed for solid tumors, according to the described criteria. FIGS. 7A-7B show single gene expression intensities and cross-gene correlation analysis for a representative FGES from each of the tumor cytokine signature sections (FIG. 6). All the presented signatures demonstrate appropriate gene expressions and inter-gene correlation coefficients. FIG. 7C shows the concordance between ssGSEA scores of FGES describing recruitment of different cell types (lymphocytes, neutrophils, CAFs) and the results of cell type deconvolution analysis. The lymphocyte recruitment signature score has the highest correlation with the predicted percentages of T cell and B cell subtypes. The neutrophil recruitment signature has the highest correlation with the predicted percentage of neutrophils. And the CAF recruitment signature has the highest correlation with the predicted percentage of fibroblasts. In FIG. 7D shows that FGES can differentiate the samples of interest: the highest pro-inflammatory signature score was observed in serum samples taken 6 hours after Varicella zoster vaccination, while the highest M1 polarization signature score was observed in serum samples taken day 2 after Varicella zoster vaccination, which corresponds to the inflammatory reaction of the organism and realization of the type 1 immune response. At the same time, the angiogenesis induction signature score decreased with the development of inflammation, which makes sense because it is related to the tissue growth and wound healing, which is an opposite process of inflammation.

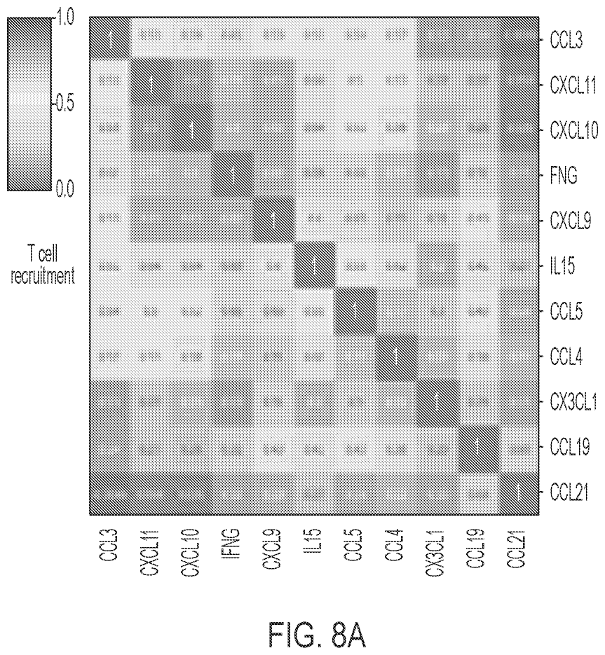

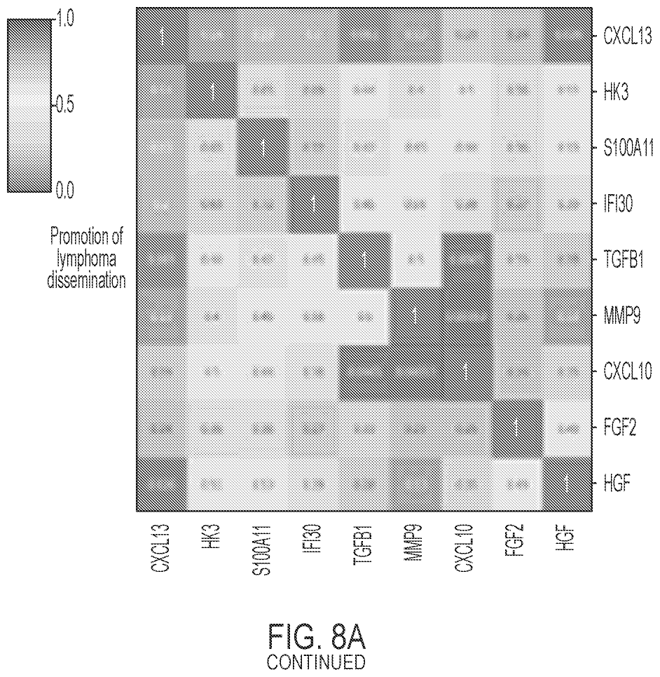

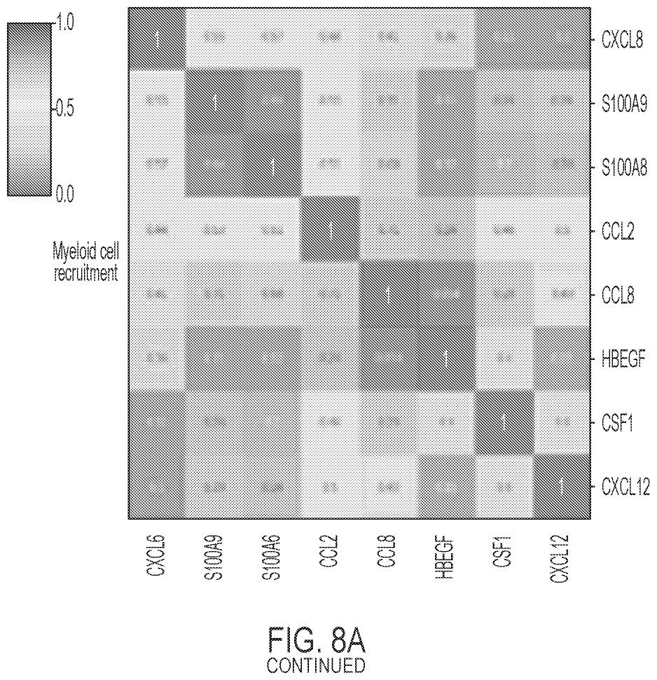

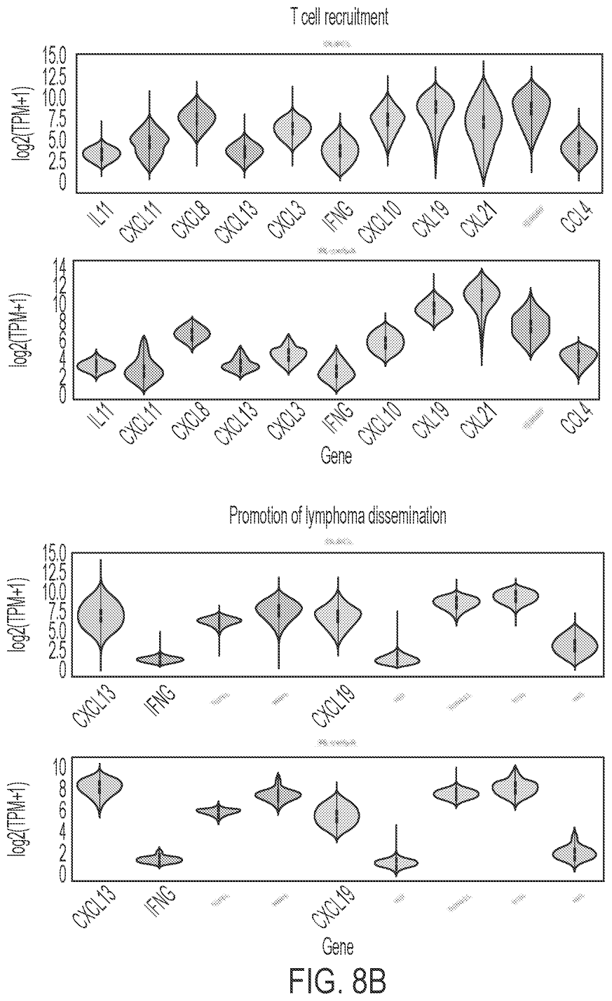

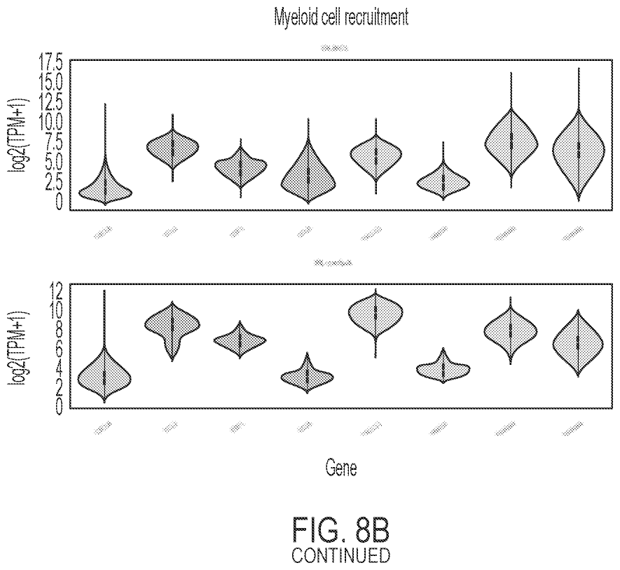

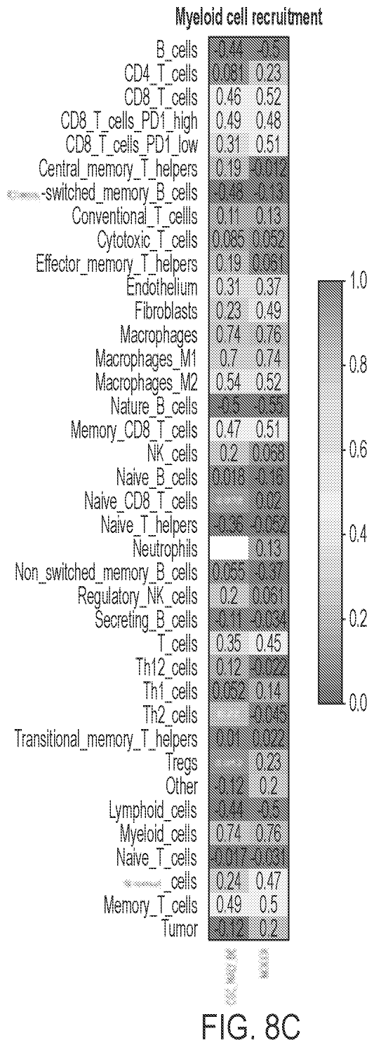

FIGS. 8A-8B show similar results for the FGES developed for hematological cancers (e.g., DLBCL and FL). As shown for the representative signatures from different sections of the tumor cytokine gene signature or cytokine gene expression, hematological FGES were also checked for adequate gene expressions and positive inter-gene correlations (FIGS. 8A-8B). Also, cell recruitment signature scores correlated with the results of cell type deconvolution analysis. For example, myeloid cell recruitment signature score has the highest correlation with macrophage populations and myeloid cells (FIG. 8C).

### Cytokine Signature Scores Differ in Normal Tissues and Cancer

Due to the different structure of body tissues and their different function, tissue infiltration by immune cells and immune cell responses are variable. These processes are regulated by distinct sets of cytokines and chemokines. To demonstrate the ability of the developed signatures to detect differences between normal tissues, FGES scores were compared between distinct tissue samples. A meta-cohort was collected with samples from different healthy tissues (blood, lymph nodes, organs) from public and internal data. RNA-Seq data for all samples were obtained using polyA enrichment of mRNA.

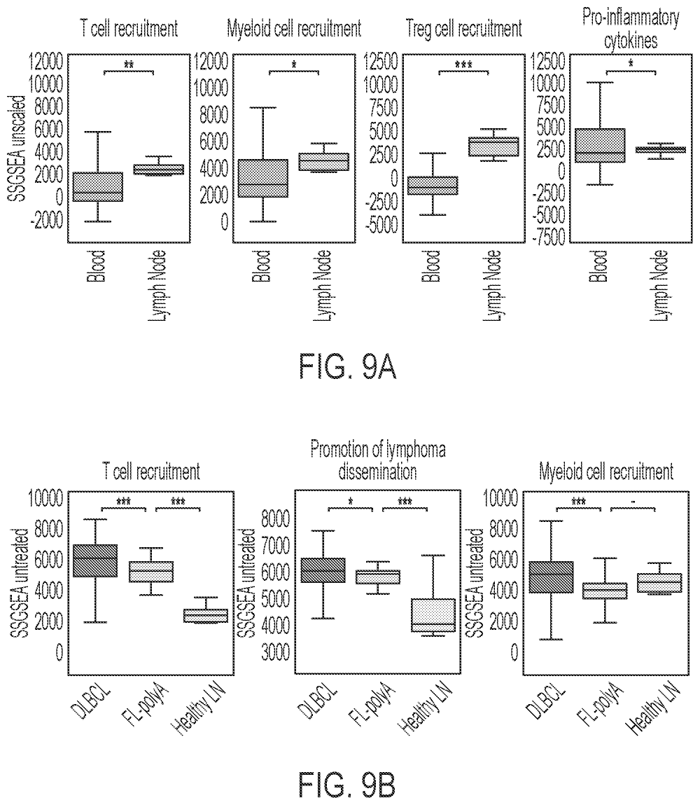

Significant differences in FGES score intensities were identified, in concordance with the expected biological properties of the analyzed tissues. In particular, myeloid, T cell, and T regulatory cell recruitment signatures were increased in normal lymph node samples compared to normal blood samples (FIG. 9A). This observation reflects lymph node function related to high traffic of immune cells through this organ. The scores of other signatures, reflecting inflammation, pro-tumor, and anti-tumor effects, were similar in the healthy blood and healthy lymph nodes, as expected.

Then the signatures in DLBCL and FL lymph node biopsies and healthy lymph node samples were compared. As expected, due to the known functional shifts in the malignant lymph node tissue, almost all of the FGESs (describing cell recruitment, pro-tumor and anti-tumor immune processes) had increased scores in the tumor samples compared to the healthy lymph nodes. Myeloid cell recruitment signature was the only one which did not differ significantly—possibly, due to the limited role of myeloid cells in the malignant lymph node inflammation (FIG. 9B).

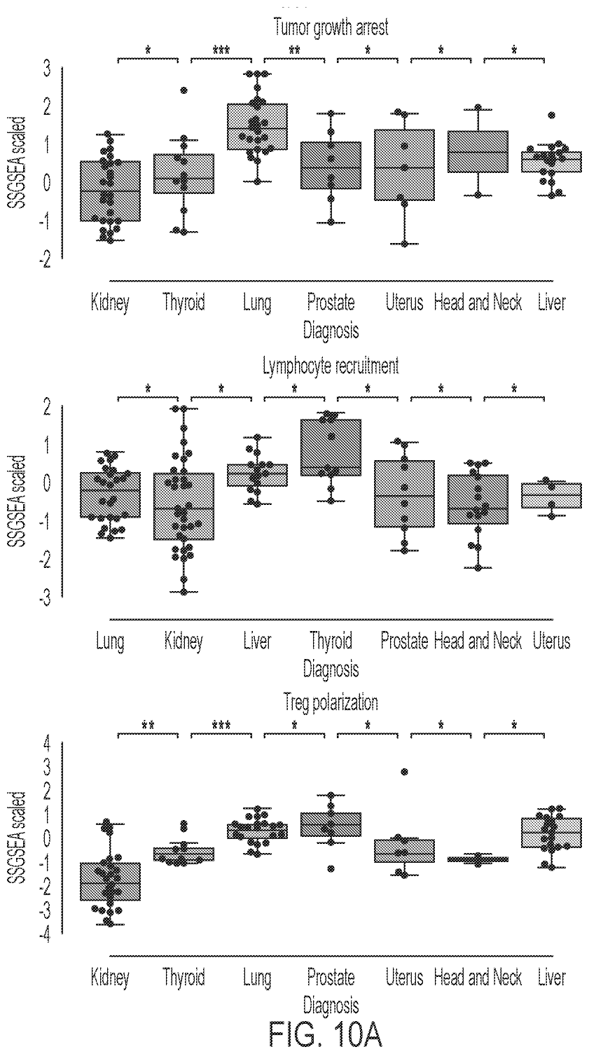

Further, FGES performance was tested in normal samples from different tissues from the TCGA cohorts. FIG. 10A-10B show the representative results showing that signature scores tend to highlight the specific features of certain tissues. For instance, the highest expression of the lymphocyte recruitment signature in the thyroid tissue compared to other sites was observed. The highest expression of the tumor growth arrest signature was registered in the lung tissue samples pointing out a high level of defense against cell damage and abnormal cell growth in a tissue contacting with oxygen and its reactive compounds. The lowest expression of the Treg polarization signature was registered in the kidney tissue samples, which reflects low levels of immune infiltration and overall inflammation characteristic for kidneys (FIG. 10A).

Next, FGES scores in the tumor and healthy samples were compared using the TCGA cohorts where normal samples are available. It was found that certain signatures reflect biological differences in a normal and tumor sample (FIG. 10B). For instance, the tumor growth arrest signature score was increased in almost all healthy tissues in comparison with tumor samples, except for the kidney cohort's kidney renal papillary cell carcinoma (KIRP) and Kidney Renal Clear Cell Carcinoma (KIRC), which confirms the suppression of abnormal cell growth mechanisms in the majority of solid cancers. In kidney, due to its biology, inflammation and tissue damage are limited, but these processes significantly increase in cases of malignancy, supporting the upregulation of the mechanisms attenuating inflammation and tissue growth.

In contrast, Treg polarization signature score was increased mostly in tumor tissues with immune suppression mechanisms. Similar scores in healthy and cancer samples—or a significantly upregulated score in healthy tissue—were registered in locations where the presence of Treg cells is important for normal function of an organ, i.e., in the prostate, gastrointestinal tract, lungs, and liver.

The score of the lymphocyte recruitment signature varied between healthy and tumor samples in different locations. A higher score in normal state was mostly registered in tissues where immune infiltration is already high in healthy conditions (thymus, gastrointestinal tract, and liver), while a higher score in tumor state was mostly registered in commonly low-infiltrated locations (esophagus, head and neck, breast, and kidney).

This analysis showed that the developed signatures are able to show significant differences between different tissues, tumor, and healthy samples and can reflect known immune and biological processes.

### Cytokine Signature Performance in Different Cancer Types

Further, the performance of the developed FGES was analyzed in different solid and hematological tumor cohorts. DLBCL sample expression data were taken from the TCGA project and NCICCR project. The inter-correlations of the developed cytokine signatures (for all solid cancers and DLBCL), their associations with the TME subtypes (for stomach adenocarcinoma and lung adenocarcinoma), and with the CMS subtypes (for colon adenocarcinoma) were examined. Prognostic value of FGES was also estimated.

The FIG. 11 correlation heatmap shows the relationships between the developed FGES analyzed on the pan-cancer cohort. The observed correlation patterns correspond to the expected biological interconnections of the processes described by the cytokine signatures: lymphocyte and myeloid cell recruitment correlate with T and B cell activation, M1 and Treg polarization, TLS formation; Th2 response and granulocyte recruitment correlate with pro-inflammatory cytokines, promotion of EMT and metastasis; and M2 polarization, CAF recruitment and stromal suppressive factors correlate with cytotoxic cell exclusion and induction of angiogenesis and tumor growth.

Tumor-infiltrating effector immune cells like CD8+ T cells and NK cells, apart from inducing cancer cells apoptosis, produce cytokines which contribute to macrophage reprogramming, recruitment of more immune cells, and development of type 1 inflammation. At the same time, the inflammatory microenvironment stimulates Treg polarization. On the other hand, recruitment and activation of granulocytes leads to escalation of myeloid-cell-derived inflammation (mostly CXCL8-, IFNa-, IFNb-, TNF-, IL-1-mediated), which interferes type 1 response and supports tumor invasion and growth. The recruitment and activation of stromal cells (CAFs, endothelium) favors the formation of suppressive TME and effector cell exclusion.

### Cytokine Signature Scores Correspond to CMS Subtypes in Colon Adenocarcinoma

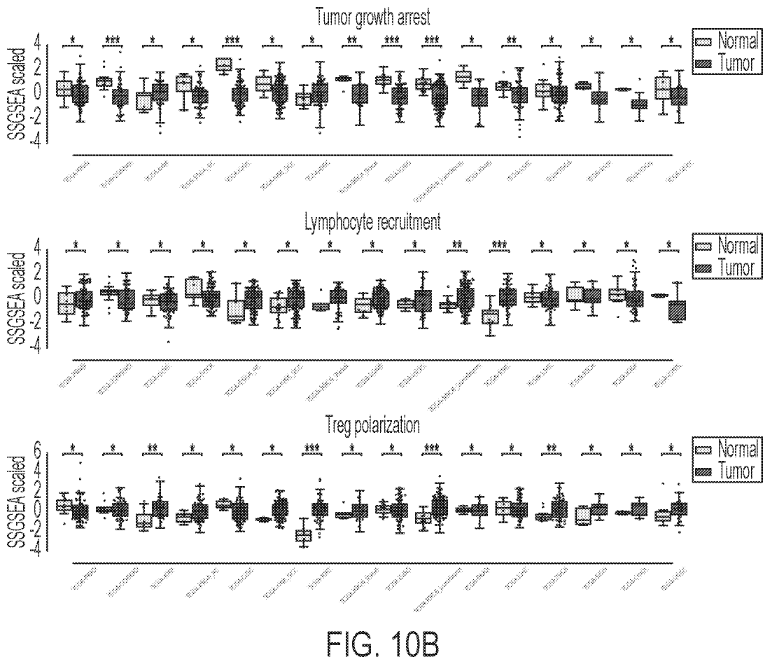

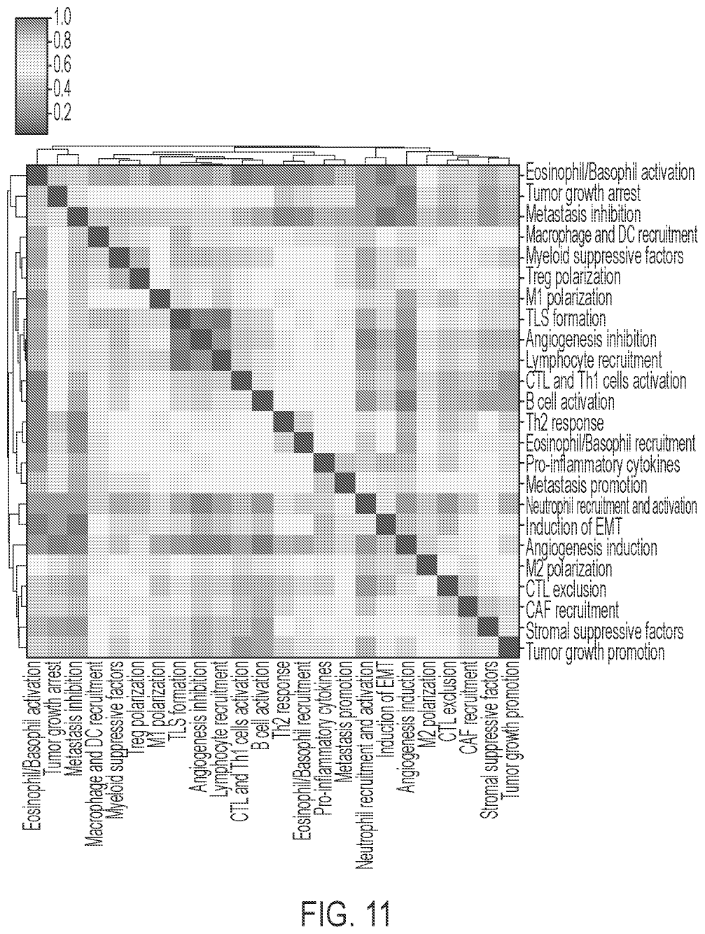

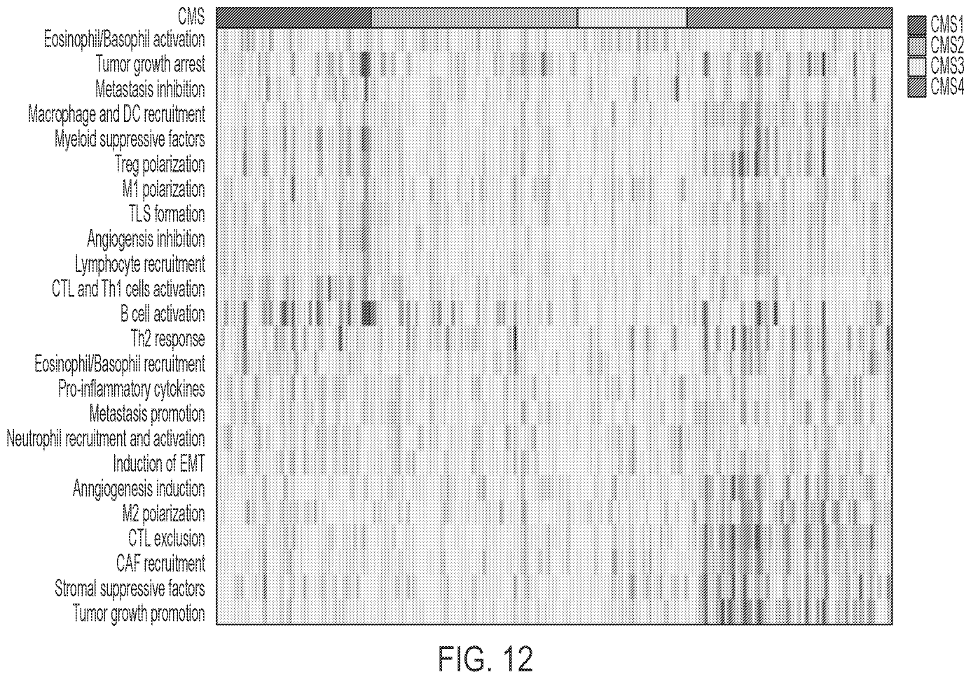

Cytokine gene signature scoring was applied to the 239 colorectal samples from the TCGA project, which have been analyzed using consensus molecular subtypes (CMS) markup. CMS subtypes showed different saturation of cytokine FGES score intensities (FIG. 12) than the CMS markup. In the CMS4 (mesenchymal) subtype, there was a higher signal from the signatures associated with stroma activation and immune suppression and tumor growth promotion, while in the CMS1 subtype (immune) the highest signal was from the signatures describing immune cell recruitment and activation. Tumor-cell-enriched subtypes CMS2 and CMS3 had overall low cytokine signature scores demonstrating low level of inflammation.

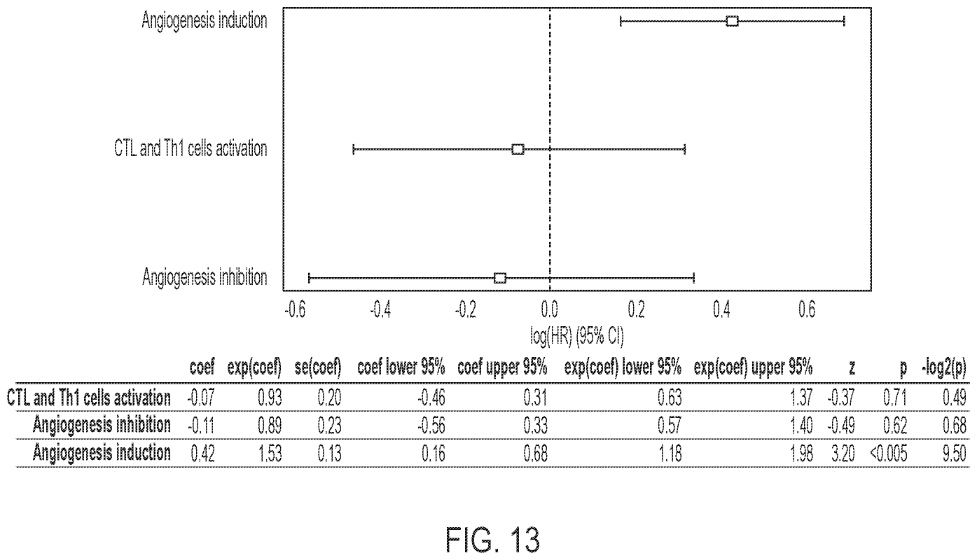

In the same COAD cohort, cox regression analysis based on the OS data for the representative signatures describing anti-tumor and pro-tumor processes was also performed. Signatures were chosen from different modules of the tumor cytokine signatures: “Angiogenesis inhibition” and “CTL and Th1 activation” from the Anti-tumor effects module, and “Angiogenesis induction” from the Pro-tumor effects module (FIG. 13). The score of the signatures describing anti-tumor processes were associated with better survival, while the score of the pro-tumor signature were associated with lower survival.

### Cytokine Signature Scores Differentiate Molecular Functional Portrait Types in Stomach Adenocarcinoma Patients

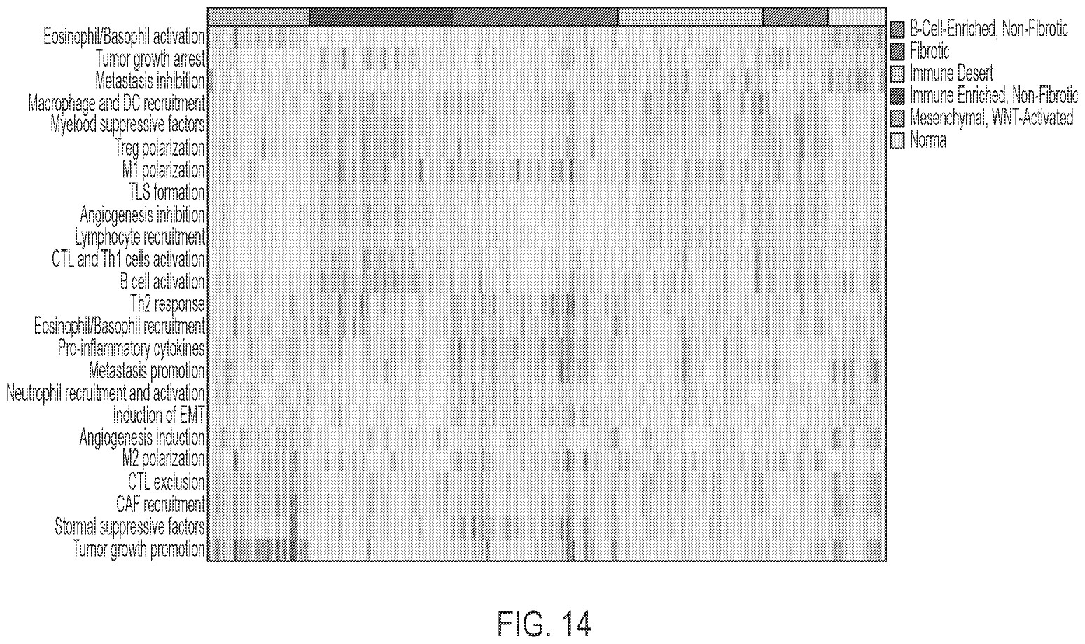

Stomach adenocarcinoma (STAD) patients can be classified into five TME Molecular Functional Portrait (MFP) types, acquired through clusterization of TCGA STAD samples as described in the International PCT Application Serial No. PCT/US2022/019538, published as International Publication Number WO2022/192393, on Sep. 15, 2022, the entire contents of which are incorporated by reference herein. Cytokine FGES was developed for solid neoplasms to 383 STAD samples and 36 normal samples from the TCGA cohort. Results showed that the signatures describe different cytokine profiles in distinct MFP subtypes of stomach cancer in concordance with their biology (FIG. 14). The mesenchymal subtype had higher scores of the cytokine FGES describing stroma activation, angiogenesis induction, anti-tumor response suppression, and granulocyte activation. The immune-enriched and B-cell-enriched subtypes demonstrated higher scores of the cytokine FGES describing effector cell recruitment and activation, and tumor suppression. The fibrotic subtype had higher scores of cytokine FGES related to stroma activation, myeloid cell recruitment (including granulocytes) and development of myeloid non-productive inflammation, favoring tumor growth, invasion and metastasis. The desert subtype had overall low intensities of all cytokine signature scores. Healthy tissue samples also had a specific cytokine signature score pattern, with the highest scores of FGESs describing the suppression of tumor growth and invasion and moderate scores of FGES related to stroma function.

### Cytokine Signature Performance in Hematological Malignancies

Lymphomas develop and progress in specialized niches such as secondary lymphoid organs, mostly in lymph nodes. The lymphoma microenvironment includes a heterogeneous population of stromal cells, such as fibroblastic reticular cells, follicular dendritic cells, nurse-like cells and mesenchymal stem cells. These cells interact with the lymphoma cells to promote lymphoma cells growth and survival, as well as interfering with the anti-tumor immune response. Due to significant differences in biology of the lymph node and other solid organs and subsequent differences in the interactions between tumor cells (stroma and immune cells) a specific set of FGES describing cytokine signaling patterns in lymphoma microenvironment was developed (Table 2).

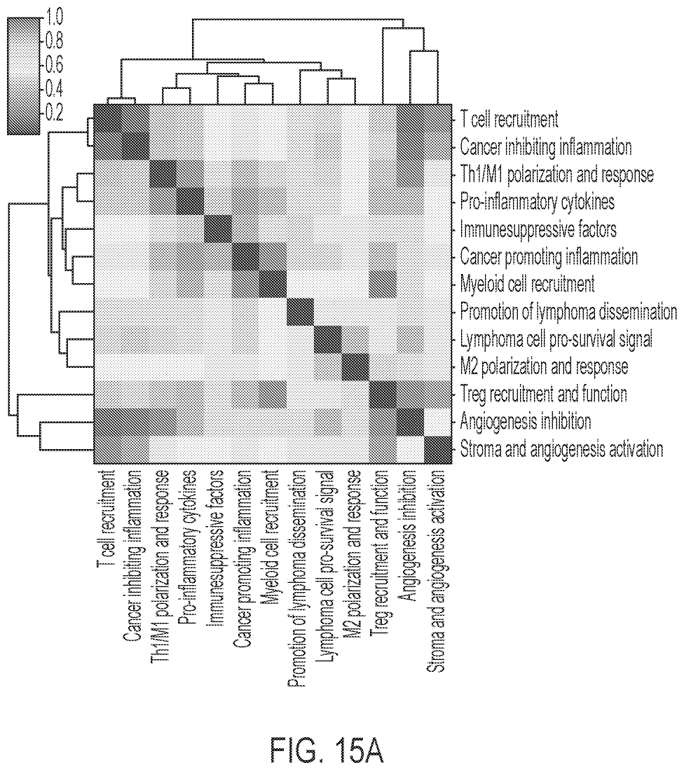

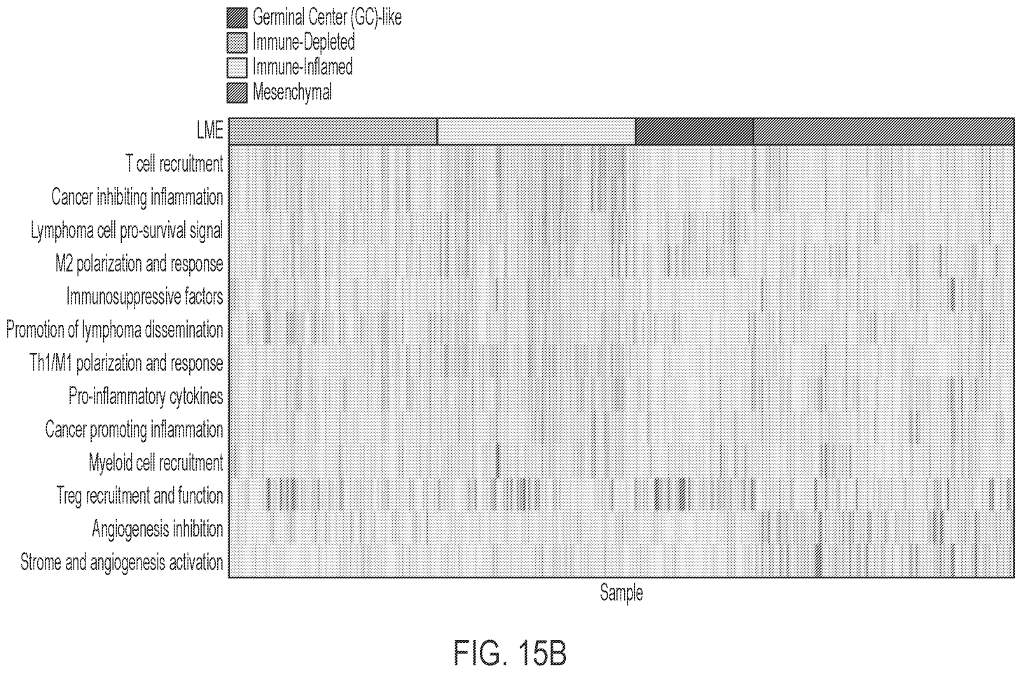

Correlation analysis of the developed FGES, performed on a DLBCL meta-cohort consisting of TCGA and NCICCR samples, showed significant positive correlations between them (FIG. 15A), indicating close interactions between the anti-tumor and pro-tumor processes and the participating cytokines in the lymph node.

Next, the expression of the developed FGES in the DLBCL TME subtypes acquired previously was determined (Kotlov et al. Cancer Discov. 1 Jun. 2021; 11 (6): 1468-1489. https://doi.org/10.1158/2159-8290.CD-20-0839). The pattern of FGES score intensities differed between the TME subtypes (FIG. 15B) according to their biology. Mesenchymal subtype was characterized by the highest scores of the stroma-associated signatures and moderate scores of signatures related to myeloid cell recruitment, suppressive factors production, promotion of tumor cell growth and dissemination. Germinal center subtype had high scores of the FGES describing lymphoma cell pro-survival signaling, M2 and Treg polarization and function. Immune-inflamed subtype was characterized by very high scores of all FGES related to inflammation, both pro-tumor and anti-tumor, but low scores of stroma-associated FGES. Immune-depleted subtype, similar to the desert subtype in solid cancers, had low intensities of all cytokine signature scores indicating absence of any inflammatory response.

Correspondence of the Cytokine Signature Scores with Tumor Architecture Mapping

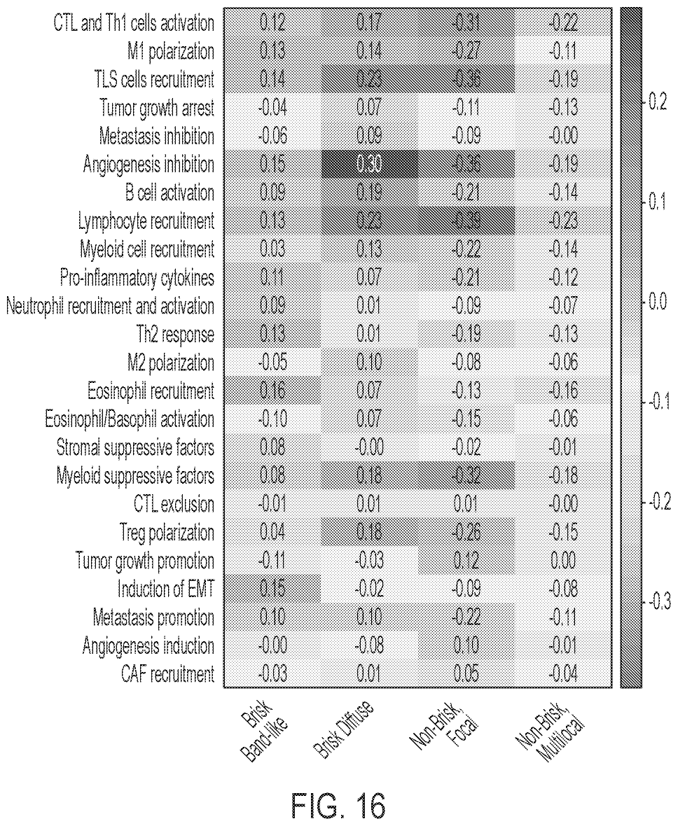

To examine the possibility of estimating the spatial architecture of a tumor using cytokine signatures, the expression scores of the developed FGES (a set for solid tumors) in the TCGA samples, which have undergone histological analysis, were typed according to the tumor infiltrating lymphocyte (TILs) distribution inside the tumor (FIG. 16). Results showed that Brisk Diffuse samples (with abundant TILs, distributed diffusely) had overall high signal intensity from most of the signatures, and had the highest scores of the FGES related to lymphocyte and myeloid cell recruitment and activation, inhibition of tumor growth, angiogenesis and metastasis, and Treg polarization compared to other sample types. Brisk Band-like samples (where TILs are abundant, but mostly gathered around fibrotic capsule and poorly interact with the tumor) were characterized by the highest scores of the signatures describing stromal suppressive factors, EMT induction, granulocyte recruitment and pro-inflammatory signaling, which indicated the presence of activated, possibly senescent, fibrotic stroma and granulocyte-mediated inflammation. Scores of the FGES related to inhibition of tumor growth, metastasis and angiogenesis were lower in this group compared to the Brisk Diffuse group.

Non-Brisk samples demonstrated low intensities of cytokine signature scores, in concordance with poor immune cell infiltration. Additionally, Non-Brisk, Focal samples with the lowest infiltration were characterized by the highest scores of the FGES describing CAF recruitment, angiogenesis induction and tumor growth promotion, indicating a high stromal component in absence of any inflammation.

Thus, this work demonstrates that the developed cytokine signatures allow samples with distinct histology to be differentiated. This work also demonstrates that cytokine signature scores may indirectly provide information about the tumor architecture.

Cytokine Signatures Complement the Four MFP Types when Characterizing a Patient's Tumor Microenvironment

To demonstrate the applicability of the cytokine signatures to tumor patient TME typing, four patients were chosen with different MFP types from the TCGA-LUAD cohort and calculated ssGSEA scores of a set of cytokine FGES developed for solid tumors.

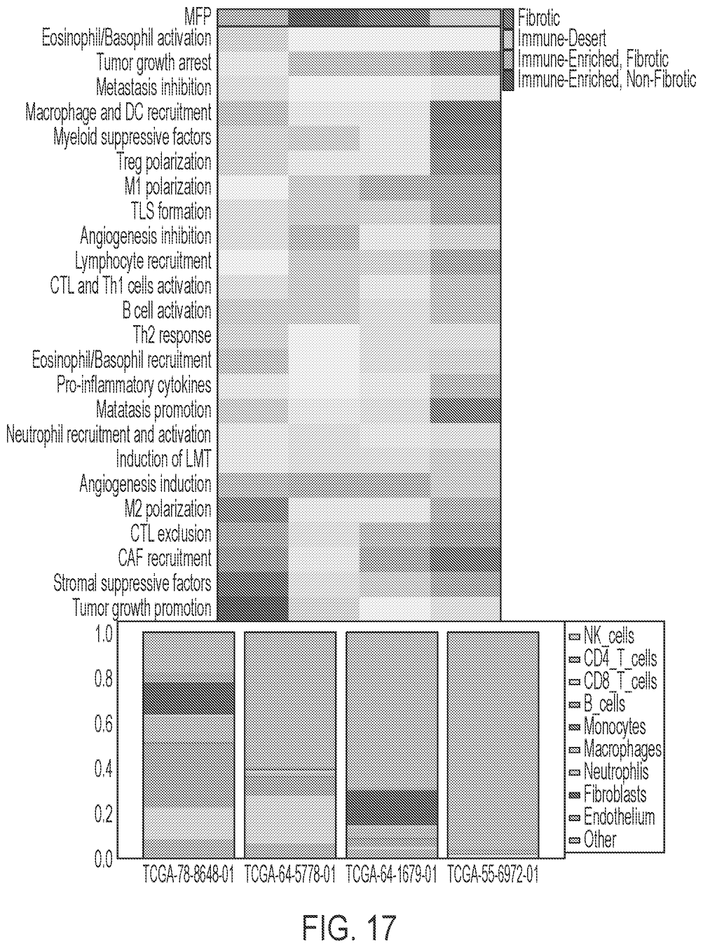

As seen in FIG. 17, the intensities of the signature scores from the Tumor Cytokine Signature correspond to the TME biological and functional characteristics described for the existing MFP types and augment MFP tumor subtyping:

Immune-Enriched, Fibrotic. The immune-enriched, fibrotic subtype is characterized by high levels of immune infiltration and low prevalence of malignant cells. This subtype is immune inflamed and has a high prevalence of stromal and fibrotic elements. Cancer-associated fibroblasts (CAF) are abundant. This subtype is commonly associated with intense vascularization and low tumor proliferation rate. The Immune-Enriched, Fibrotic subtype is characterized by very high level of cytokines stimulating stroma activation, angiogenesis, CTL exclusion, M2 recruitment and polarization. Also, high levels of cytokines favoring immune cell recruitment and activation were observed.

Immune-Enriched, Non-fibrotic. The immune-enriched, non-fibrotic subtype is characterized by high levels of immune infiltration, including cytotoxic effector cells, and low prevalence of stromal and fibrotic elements. This subtype is immune inflamed and has high tumor mutational burden (TMB). The Immune-Enriched, Non-Fibrotic subtype is characterized by high abundance of cytokines responsible for lymphocyte recruitment and activation, inhibition of angiogenesis and tumor growth. Stroma-associated and granulocyte-associated cytokines are low.

Fibrotic. The fibrotic subtype is characterized by minimal immune infiltration and high prevalence of stromal elements, often with dense collagen formation and intense angiogenesis. This subtype is immune non-inflamed. Cancer-associated fibroblasts (CAF) are abundant. TGF-β signaling pathway is often upregulated. Signs of epithelial-mesenchymal transition (EMT) are present. The Fibrotic subtype is characterized by a high abundance of cytokines responsible for fibroblast recruitment and activation, angiogenesis induction, and CTL exclusion. Also, moderate levels of cytokines associated with myeloid cell recruitment and activation (including granulocytes), promotion of EMT, metastasis and tumor growth are observed, which may favor tumor dissemination and treatment resistance.

Immune Desert. The immune desert subtype is characterized by a high percentage of malignant cells, while immune infiltration is only minimal or completely absent. Immune non-inflamed. This subtype also demonstrates increased chromosomal instability (CIN). High tumor proliferation rate is commonly observed. The Immune-Desert subtype lacks any cytokine signaling due to the absence of cytokine-producing cells.

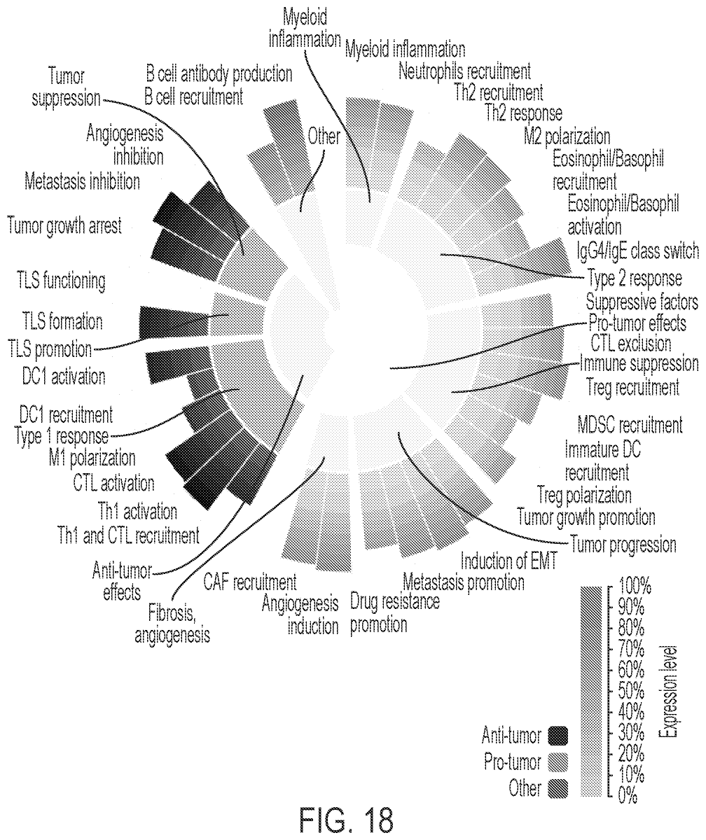

As another confirmation of the accuracy of cytokine expression measurements, FIG. 17 shows that cytokine expression may correlate with biological function. For example, cytokines associated with myeloid inflammation (pro-inflammatory response and neutrophil recruitment and activation) are positively correlated to one another. This suggests that the cytokines expression profiles are relevant to and predictive of biological processes and pathways. FIG. 18 shows a cytokine signature of a subject having cutaneous melanoma.

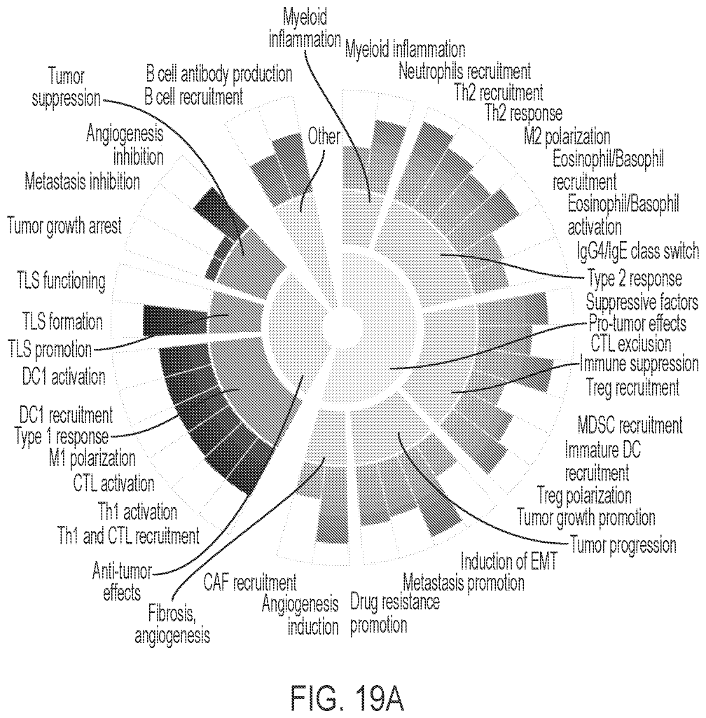

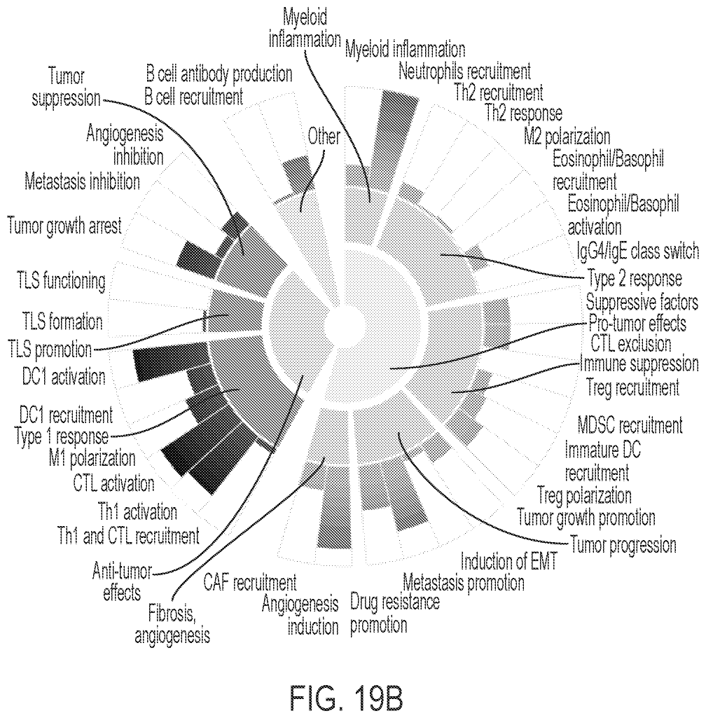

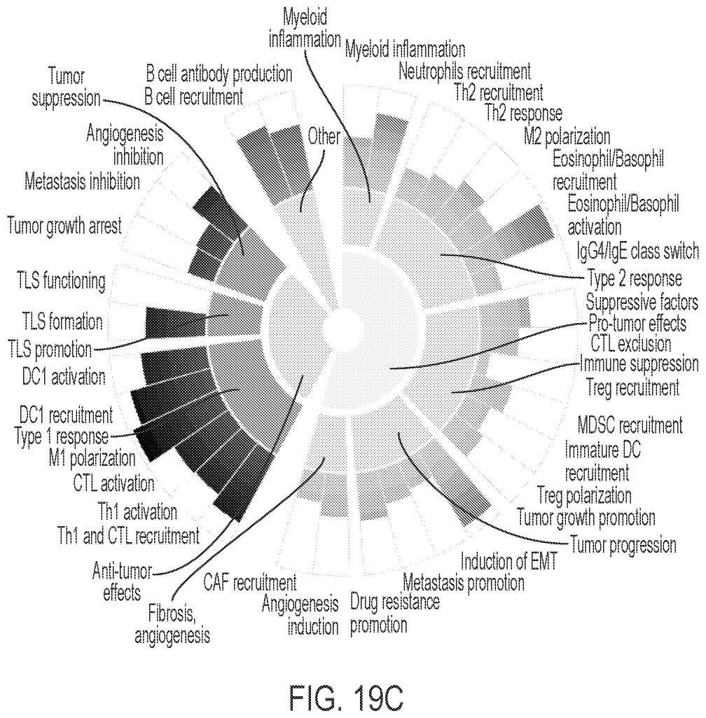

Cytokine expression can identify potential personalized therapies in subjects having the same TME tumor type. For example, FIGS. 19A-19C show cytokine expression solar diagrams for three different subjects all having immune-enriched, Non-Fibrotic Molecular Functional Portrait (MFP) tumor micro-environment type (TME). The subject associated with FIG. 19A has cytokine expression indicating moderate effector cell activation, but high level of suppressive factors and high Th2 response. Based on this, the subject of FIG. 19A is expected to respond to Immunotherapy supplemented with drugs against suppressive/Th2 factors (IL-10, IL-4, TGFb, IDO-1, etc.). The subject associated with FIG. 19B has a high level of effector-cell-activating signaling, a high level of neutrophil/angiogenesis activation, and low levels of suppressive factors. Based on this, the subject of FIG. 19B is expected to respond to Immunotherapy. The subject associated with FIG. 19A has cytokine expression indicating the highest level of effector-cell-activating signaling of the subject and high granulocyte activation.

In summary, two sets of cytokine gene signatures were developed to describe biological processes taking place in a tumor (immune cell recruitment, angiogenesis, stroma activation, etc.) for solid and hematologic malignancies. The cytokine signatures also provide a way to distinguish between normal and malignant/inflamed tissue. Cytokine gene signatures may have prognostic value in certain pathological conditions (e.g., in determining potentially effective treatments). Cytokine gene signature may be used to determine the spatial architecture of the tumor. And cytokine gene signatures and/or gene expression complements tumor subtyping of the Molecular Functional Portrait (MFP).

### Therapeutic Agent Selection Using Cytokine Signatures

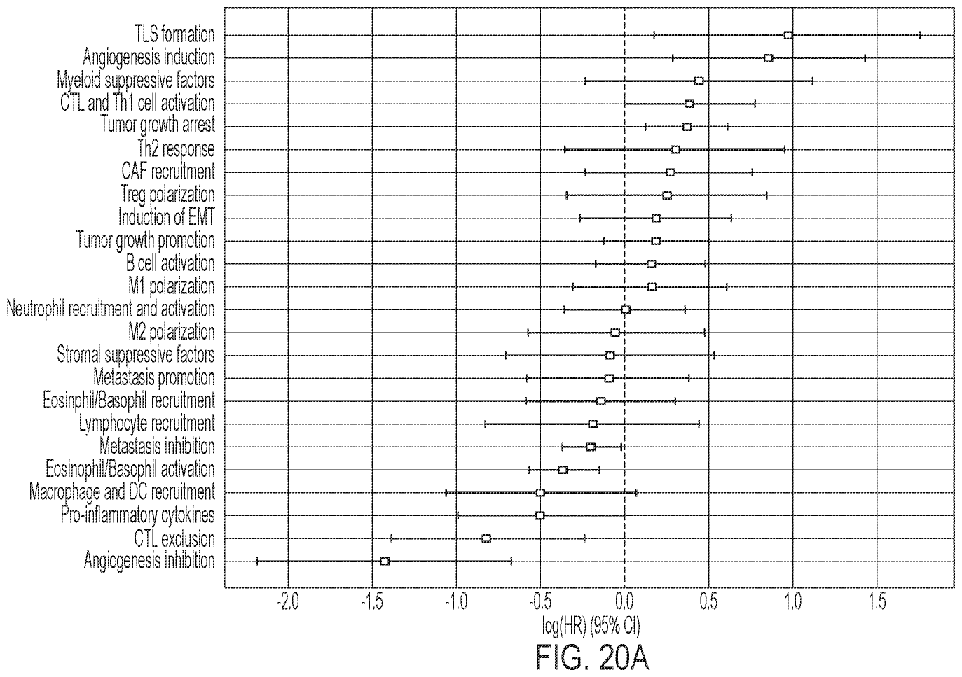

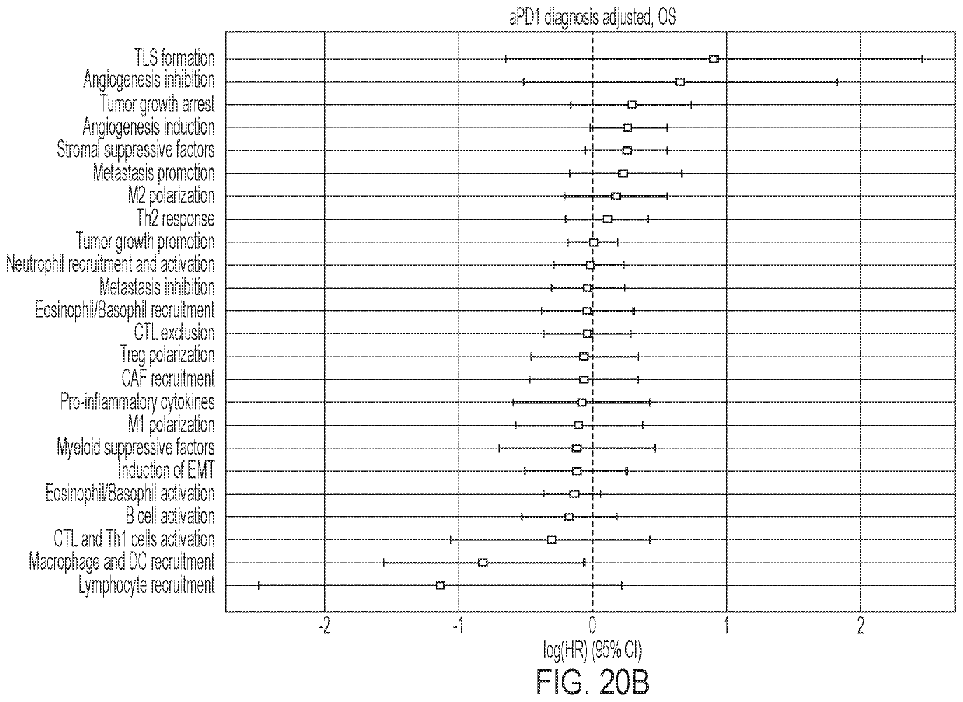

This example describes use of cytokine signatures as biomarkers of treatment response. Cox regression analysis for the cytokine signatures developed for solid tumor cancers was performed. In a Clear Cell Renal Cell Carcinoma cohort treated with tyrosine kinase inhibitors (TKI), high normalized gene group scores of TLS formation, Angiogenesis induction, CTL and Th1 activation, and Tumor growth arrest signatures were associated with significantly worse progression-free survival (PFS) and, thus, worse response to this kind of therapy (FIG. 20A). Most of these signatures are related to active Type 1 response, which is not the target of TKI, so such patients seem to be unsuitable for this therapy, and a pro-angiogenic environment further worsens the response, leveling out the effect of TKI. At the same time, high normalized gene group scores of Angiogenesis inhibition, CTL exclusion, Pro-inflammatory cytokines, Eosinophils/Basophil activation and Metastasis inhibition were favorable factors of longer PFS and, thus, better response to TKI (FIG. 20A), and can be used as indicators of the most suitable patients.

Cytokine signatures in patients with Cutaneous Melanoma, Gastric Cancer, and Head and Neck Squamous Carcinoma, treated with anti-PD-1 immune checkpoint inhibitor (ICI) were also investigated. High Macrophage and DC recruitment normalized gene group scores were associated with significantly better survival, and Lymphocyte recruitment, CTL and Th1 activation and B cell activation normalized gene group scores also indicated better survival. All of these gene groups are related to effective immune cell infiltration and activation. At the same time, TLS formation, Angiogenesis inhibition, Tumor growth arrest, Angiogenesis induction, Stromal suppressive factors, Metastasis promotion and M2 polarization normalized gene group scores showed a tendency to be negative factors of overall survival. Most of those gene groups are associated with immune cell suppression and stroma activation, and these groups from the Tumor suppression sector include factors which decrease the overall cytokine content, which can alter anti-tumor immunity too.

Patients with high normalized gene group scores for activation of Immune cell recruitment, B cell response, and Type 1 response of STC cytokine signatures, and with low normalized scores of the gene groups associated with tumor suppression, immune suppression, and stroma activation, are likely to demonstrate the highest response to ICI therapy. On the other hand, patients with high gene group scores in the sectors of Tumor suppression, and Myeloid inflammation, and with low normalized gene group scores in the sectors of Type 1 response and Fibrosis, Angiogenesis should be considered potentially responsive to TKI therapy.

### EQUIVALENTS

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

1. A method for generating a cytokine signature for a subject having solid tumor cancer, the method comprising:
using at least one computer hardware processor to perform:
obtaining RNA expression data for the subject, the RNA expression data for the subject indicating RNA expression levels for at least some genes in each group of at least some of a plurality of solid tumor cancer (STC) gene groups, the plurality of STC gene groups comprising (i) STC gene groups associated with pro-tumor effects, (ii) STC gene groups associated with anti-tumor effects, and (iii) STC gene groups associated with B cell effects;
obtaining RNA expression data for a cohort of subjects having a same type of STC as the subject, the RNA expression data for the cohort indicating RNA expression levels for the at least some of the genes in each group of the at least some of the plurality of STC gene groups;
generating, using the RNA expression data for the subject and the RNA expression data for the cohort of subjects, the cytokine signature for the subject, the cytokine signature comprising normalized STC gene group scores for respective STC gene groups in the at least some of the plurality of STC gene groups, the generating comprising:
determining, using the RNA expression data for the subject, initial STC gene group scores for (i) one or more STC gene groups associated with pro-tumor effects, (ii) one or more STC gene groups associated with anti-tumor effects, and/or (iii) one or more STC gene groups associated with B cell effects; and
determining the normalized STC gene group scores of the cytokine signature by normalizing the initial STC gene group scores using the RNA expression data for the cohort of subjects.

2. The method of claim 1,
wherein the STC gene groups associated with pro-tumor effects comprise one or more gene groups from among: myeloid inflammation groups, Type 2 response groups, immune suppression groups, tumor promotion groups, and stroma activation groups, and
wherein determining the initial STC gene group scores comprises, determining STC gene group scores for the one or more gene groups from among the myeloid inflammation groups, the Type 2 response groups, the immune suppression groups, the tumor promotion groups, and the stroma activation groups.

3. The method of claim 1,
wherein the STC gene groups associated with anti-tumor effects comprise one or more gene groups from among: Type 1 response groups and tumor suppression groups, and
wherein determining the initial STC gene group scores comprises, determining STC gene group scores for the one or more gene groups from among the Type 1 response groups and the tumor suppression groups.

4. (canceled)

5. The method of claim 2,
wherein the myeloid inflammation groups comprise the pro-inflammatory cytokines group which comprises at least three genes listed in Table 1 for the pro-inflammatory cytokines group, and
wherein determining the initial STC group scores comprises:
obtaining, from the RNA expression data for the subject, RNA expression levels for the at least three genes listed in Table 1; and
determining an initial STC group score for the pro-inflammatory cytokines group using the RNA expression levels for the at least three genes listed in Table 1.

6. (canceled)

7. The method of claim 2,
wherein the Type 2 response groups comprise the Th2 response group which comprises at least three genes listed in Table 1 for the Th2 response group, and
wherein determining the initial STC group scores comprises:
obtaining, from the RNA expression data for the subject, RNA expression levels for the at least three genes listed in Table 1; and
determining an initial STC group score for the Th2 response group using the RNA expression levels for the at least three genes listed in Table 1.

8-10. (canceled)

11. The method of claim 2,
wherein the immune suppression groups comprise the stromal suppressive factors group which comprises at least three genes listed in Table 1 for the stromal suppressive factors group, and
wherein determining the initial STC group scores comprises:
obtaining, from the RNA expression data for the subject, RNA expression levels for the at least three genes listed in Table 1; and
determining an initial STC group score for the stromal suppressive factors group using the RNA expression levels for the at least three genes listed in Table 1.

12-14. (canceled)

15. The method of claim 2,
wherein the tumor promotion groups comprise the tumor growth promotion group which comprises at least three genes listed in Table 1 for the tumor growth promotion group, and
wherein determining the initial STC group scores comprises:
obtaining, from the RNA expression data for the subject, RNA expression levels for the at least three genes listed in Table 1; and
determining an initial STC group score for the tumor growth promotion group using the RNA expression levels for the at least three genes listed in Table 1.

16-17. (canceled)

18. The method of claim 2,
wherein the stroma activation groups comprise the angiogenesis induction group which comprises at least three genes listed in Table 1 for the angiogenesis induction group, and
wherein determining the initial STC group scores comprises:
obtaining, from the RNA expression data for the subject, RNA expression levels for the at least three genes listed in Table 1; and
determining an initial STC group score for the angiogenesis induction group using the RNA expression levels for the at least three genes listed in Table 1.

19-20. (canceled)

21. The method of claim 3,
wherein the Type 1 response groups comprise the M1 polarization group which comprises at least three genes listed in Table 1 for the M1 polarization group, and
wherein determining the initial STC group scores comprises:
obtaining, from the RNA expression data for the subject, RNA expression levels for the at least three genes listed in Table 1; and
determining an initial STC group score for the M1 polarization group using the RNA expression levels for the at least three genes listed in Table 1.

22. (canceled)

23. The method of claim 3,
wherein the tumor suppression groups comprise the tumor growth arrest group which comprises at least three genes listed in Table 1 for the tumor growth arrest group, and
wherein determining the initial STC group scores comprises:
obtaining, from the RNA expression data for the subject, RNA expression levels for the at least three genes listed in Table 1; and
determining an initial STC group score for the tumor growth arrest group using the RNA expression levels for the at least three genes listed in Table 1.

24-25. (canceled)

26. The method of claim 5,
wherein the B cell function group comprises the B cell activation group which comprises at least three genes listed in Table 1 for the B cell activation group, and
wherein determining the initial STC group scores comprises:
obtaining, from the RNA expression data for the subject, RNA expression levels for the at least three genes listed in Table 1; and
determining an initial STC group score for the B cell activation group using the RNA expression levels for the at least three genes listed in Table 1.

27-30. (canceled)

31. The method of claim 1, further comprising:
determining that each normalized STC gene group score is (i) below a first threshold value, (ii) between the first threshold value and a second threshold value, or (iii) above the second threshold value, wherein each of the first and second threshold values is determined using the RNA expression data for the cohort of subjects, and
identifying each respective normalized STC gene group score as “low” when the respective normalized STC gene group score is below the first threshold value, identifying each respective normalized STC gene group score as “medium” when the respective gene group score is between the first threshold value and the second threshold value, or identifying each respective normalized STC gene group score as “high” when the respective gene group score is above the second threshold value.

32-34. (canceled)

35. The method of claim 1, further comprising identifying at least one therapeutic agent for administration to the subject using the cytokine signature.

36. (canceled)

37. The method of claim 35, wherein the at least one therapeutic agent is an immune checkpoint inhibitor (ICI) or a tyrosine kinase inhibitor (TKI).

38. The method of claim 35, wherein the at least one identified therapeutic agent is an immune checkpoint inhibitor when the subject has been identified as having:
(i) a “high” normalized STC gene group score for one or more of the following gene groups: immune cell recruitment groups, B cell response groups, or Type 1 response groups.

39. The method of claim 35, wherein the at least one identified therapeutic agent is an immune checkpoint inhibitor (ICI) when the subject is identified as having:
(i) a “low normalized STC gene group score for one or more of the following gene groups: tumor suppression gene groups, immune suppression gene groups, or stromal activation gene groups.

40. The method of claim 35, wherein the at least one identified therapeutic agent is a TKI when the subject has been identified as having:
(i) a “high” normalized STC gene group score for one or more of the following gene groups: myeloid inflammation gene groups, or tumor suppression gene groups.

41. The method of claim 35, wherein the at least one identified therapeutic agent is a TKI when the subject is identified as having:
(i) a “low normalized STC gene group score for one or more of the following gene groups: Type 1 response gene groups or stromal activation groups.

42-75. (canceled)

76. A system comprising:
at least one computer hardware processor;
at least one non-transitory computer-readable storage medium storing processor executable instructions that, when executed by the at least one computer hardware processor, cause the at least one computer hardware processor to perform a method for generating a cytokine signature for a subject having solid tumor cancer, the method comprising:
obtaining RNA expression data for the subject, the RNA expression data for the subject indicating RNA expression levels for at least some genes in each group of at least some of a plurality of solid tumor cancer (STC) gene groups, the plurality of STC gene groups comprising (i) STC gene groups associated with pro-tumor effects, (ii) STC gene groups associated with anti-tumor effects, and (iii) STC gene groups associated with B cell effects;
obtaining RNA expression data for a cohort of subjects having a same type of STC as the subject, the RNA expression data for the cohort indicating RNA expression levels for the at least some of the genes in each group of the at least some of the plurality of STC gene groups;
generating, using the RNA expression data for the subject and the RNA expression data for the cohort of subjects, the cytokine signature for the subject, the cytokine signature comprising normalized STC gene group scores for respective STC gene groups in the at least some of the plurality of STC gene groups, the generating comprising:
determining, using the RNA expression data for the subject, initial STC gene group scores for (i) one or more STC gene groups associated with pro-tumor effects, (ii) one or more STC gene groups associated with anti-tumor effects, and/or (iii) one or more STC gene groups associated with B cell effects; and
determining the normalized STC gene group scores of the cytokine signature by normalizing the initial STC gene group scores using the RNA expression data for the cohort of subjects.

77. At least one non-transitory computer-readable storage medium storing processor executable instructions that, when executed by at least one computer hardware processor, cause the at least one computer hardware processor to perform a method for generating a cytokine signature for a subject having solid tumor cancer, the method comprising:
obtaining RNA expression data for the subject, the RNA expression data for the subject indicating RNA expression levels for at least some genes in each group of at least some of a plurality of solid tumor cancer (STC) gene groups, the plurality of STC gene groups comprising (i) STC gene groups associated with pro-tumor effects, (ii) STC gene groups associated with anti-tumor effects, and (iii) STC gene groups associated with B cell effects;
obtaining RNA expression data for a cohort of subjects having a same type of STC as the subject, the RNA expression data for the cohort indicating RNA expression levels for the at least some of the genes in each group of the at least some of the plurality of STC gene groups;
generating, using the RNA expression data for the subject and the RNA expression data for the cohort of subjects, the cytokine signature for the subject, the cytokine signature comprising normalized STC gene group scores for respective STC gene groups in the at least some of the plurality of STC gene groups, the generating comprising:
determining, using the RNA expression data for the subject, initial STC gene group scores for (i) one or more STC gene groups associated with pro-tumor effects, (ii) one or more STC gene groups associated with anti-tumor effects, and/or (iii) one or more STC gene groups associated with B cell effects; and
determining the normalized STC gene group scores of the cytokine signature by normalizing the initial STC gene group scores using the RNA expression data for the cohort of subjects.
